(19) United States
(12) Patent Application Publication
(10) Pub. No.: US 2010/0249163 A1
(43) Pub. Date: Sep. 30, 2010

(54) RENIN INHIBITORS

(76) Inventors: Daniel Dube, Saint-Lazare (CA); Erich L. Grimm, Baie d'Urfe (CA); Dwight MacDonald, L'le Bizard (CA); Bruce MacKay, Dollard-des-Ormeaux (CA)

Correspondence Address: MERCK, P O BOX 2000, RAHWAY, NJ, 07065-0907, US

(21) Appl. No.: 12/746,037

(22) PCT Filed: Nov. 26, 2008
(86) PCT No.: PCT/CA2008/002069

§ 371 Date: Jun. 3, 2010

Related U.S. Application Data
(60) Provisional application No. 61/005,223, filed on Dec. 4, 2007.
(60) Provisional application No. 61/065,087, filed on Feb. 8, 2008.

(51) Int. Cl. A61K 31/513; A61K 31/4545; A61K 31/454; A61K 31/451; C07D 401/04; C07D 417/04; C07D 211/52; A61P 9/10; A61P 27/06; A61P 9/04; A61P 5/42; A61P 13/12; A61P 9/12; A61P 15/10; A61P 25/22

(52) U.S. Cl. 514/274; 514/318; 514/321; 514/326; 514/327; 544/311; 546/194; 546/198; 546/209; 546/242

(57) ABSTRACT

The present invention relates to piperidinyl-based renin inhibitor compounds having the formula

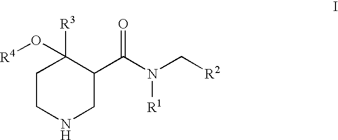

containing amino-terminal groups, and their use in treating cardiovascular events and renal insufficiency.

### JOINT RESEARCH AGREEMENT

The claimed invention was made as a result of activities undertaken within the scope of a joint research agreement between Merck & Co., Inc. and Actelion Pharmaceuticals Ltd. The agreement was executed on Dec. 4, 2003. The field of the invention is described below.

### FIELD OF THE INVENTION

The invention relates to novel renin inhibitors of the general formula (I). The invention also concerns related aspects including processes for the preparation of the compounds, pharmaceutical compositions containing one or more compounds of formula (I) and especially their use as renin inhibitors in cardiovascular events and renal insufficiency.

### BACKGROUND OF THE INVENTION

In the renin-angiotensin system (RAS) the biologically active angiotensin II (Ang II) is generated by a two-step mechanism. The highly specific enzyme renin cleaves angiotensinogen to angiotensin I (Ang I), which is then further processed to Ang II by the less specific angiotensin-converting enzyme (ACE). Ang II is known to work on at least two receptor subtypes called AT₁ and AT₂. Whereas AT₁ seems to transmit most of the known functions of Ang II, the role of AT₂ is still unknown.

Modulation of the RAS represents a major advance in the treatment of cardiovascular diseases. ACE inhibitors and AT₁ blockers have been accepted to treat hypertension (Waeber B. et al., “The renin-angiotensin system: role in experimental and human hypertension”, in Birkenhager W. H., Reid J. L. (eds): Hypertension, Amsterdam, Elsevier Science Publishing Co, 1986, 489-519; Weber M. A., Am. J. Hypertens., 1992, 5, 247S). In addition, ACE inhibitors are used for renal protection (Rosenberg M. E. et al., Kidney International, 1994, 45, 403; Breyer J. A. et al., Kidney International, 1994, 45, S156), in the prevention of congestive heart failure (Vaughan D. E. et al., Cardiovasc. Res., 1994, 28, 159; Fouad-Tarazi F. et al., Am. J. Med., 1988, 84 (Suppl. 3A), 83) and myocardial infarction (Pfeffer M. A. et al., N. Engl. J. Med., 1992, 327, 669).

The rationale to develop renin inhibitors is the specificity of renin (Kleinert H. D., Cardiovasc. Drugs, 1995, 9, 645). The only substrate known for renin is angiotensinogen, which can only be processed (under physiological conditions) by renin. In contrast, ACE can also cleave bradykinin besides Ang I and can be by-passed by chymase, a serine protease (Husain A., J. Hypertens., 1993, 11, 1155). In patients inhibition of ACE thus leads to bradykinin accumulation causing cough (5-20%) and potentially life-threatening angioneurotic edema (0.1-0.2%) (Israili Z. H. et al., Annals of Internal Medicine, 1992, 117, 234). Chymase is not inhibited by ACE inhibitors. Therefore, the formation of Ang II is still possible in patients treated with ACE inhibitors. Blockade of the AT₁ receptor (e.g. by losartan) on the other hand overexposes other AT-receptor subtypes (e.g. AT₂) to Ang II, whose concentration is significantly increased by the blockade of AT₁ receptors. In summary, renin inhibitors are expected to demonstrate a different pharmaceutical profile than ACE inhibitors and AT₁ blockers with regard to efficacy in blocking the RAS and in safety aspects.

The present invention relates to the identification of renin inhibitors of a non-peptidic nature and of low molecular weight. Described are orally active renin inhibitors of long duration of action which are active in indications beyond blood pressure regulation where the tissular renin-chymase system may be activated leading to pathophysiologically altered local functions such as renal, cardiac and vascular remodeling, atherosclerosis, and possibly restenosis. So, the present invention describes these non-peptidic renin inhibitors.

The compounds described in this invention represent a novel structural class of renin inhibitors.

### SUMMARY OF THE INVENTION

The present invention is directed to certain compounds and their use in the inhibition of the renin enzyme, including treatment of conditions known to be associated with the renin system. The invention includes compounds of Formula I:

The present invention relates to compounds of the formula (I)

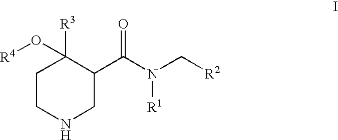

wherein
R¹ is selected from the group consisting of C₁₋₆alkyl or C₃₋₈cycloalkyl;
R² is an aryl ring, a 5 or 6-membered heteroaryl ring containing 1, 2, 3 or 4 heteroatoms selected from N, O or S, or a fused 9 or 10-membered heteroaryl ring system containing 1, 2, 3 or 4 heteroatoms selected from N, O or S, wherein said aryl or heteroaryl ring is unsubstituted or mono-, di-, tri- or tetra-substituted with a group independently selected from

1) halogen,

2) O—C₁₋₅alkylene-O—C₁₋₅alkyl,

3) C₁₋₅ alkylene-O—C₁₋₅alkyl,

4) C₁₋₅alkylene-N(C₁₋₅alkyl)-C(O)—C₁₋₅alkyl,

5) C₁₋₅alkylene-NH—C(O)—C₁₋₅alkyl, and

6) oxo;

R³ is an aryl ring, a 5 or 6-membered heteroaryl ring containing 1, 2, 3 or 4 heteroatoms selected from N, O or S, a fused 9 or 10-membered heteroaryl ring system containing 1, 2, 3 or 4 heteroatoms selected from N, O or S, or C₃₋₈ cycloalkyl, wherein said aryl ring, heteroaryl ring, or C₃₋₈ cycloalkyl is unsubstituted or mono-, di-, tri- or tetra-substituted with a group independently selected from

1) halogen,

2) C₁₋₅alkoxy,

3) CF₃,

4) NH₂,

5) O—(C₁₋₅alkylene)-aryl,

6) C₁₋₅ alkyl,

7) oxo,

R⁴ is selected from the group consisting of

hydrogen,

C₁₋₅alkyl,

C₁₋₅alkylene-aryl,

C₁₋₅alkylene-O—C₁₋₅alkyl,

C₃₋₈cycloalkyl,

C₁₋₅alkyleneNHC(O)—C₁₋₅alkyl,

C(O)—O—C₁₋₅alkyl, and

C₁₋₅alkylene-heteroaryl,

wherein aryl is unsubstituted or mono- or di-substituted with halogen, alkyl is unsubstituted or mono- or di-substituted with OH, and heteroaryl is a 5 or 6 membered unsaturated ring containing 1, 2, 3 or 4 heteroatoms selected from the group consisting of N, O and S;
or a pharmaceutically acceptable salt thereof.

### DETAILED DESCRIPTION OF THE DISCLOSURE

In one embodiment of compounds of formula I,

R¹ is cyclopropyl, and all other variables are as previously defined.

In another embodiment of compounds of formula I,

R² is phenyl, pyridine, pyrimidine or indole, unsubstituted or mono-, di-, tri- or tetra-substituted with a group independently selected from

1) Cl,

2) O(CH₂)₂OCH₃,

3) (CH₂)₂₋₃OCH₃,

4) CH₂N(CH₃)C(O)CH₃, and

5) oxo;

and all other variables are as previously defined.

In another embodiment of compounds of formula I,

R³ is phenyl, pyridinyl, thiazole, imidazole or benzoxazole, unsubstituted or mono-, di-, tri- or tetra-substituted with a group independently selected from

1) Cl,

2) F,

3) C₁₋₄alkoxy,

4) CF₃,

5) NH₂,

6) OCH₂phenyl,

7) C₁₋₄ alkyl,

8) oxo;

and all other variables are as previously defined.

In another embodiment of compounds of formula I,

R⁴ is selected from the group consisting of

hydrogen,

C₁₋₅alkyl,

CH₂fluorophenyl,

(CH₂)₂OCH₃,

CH₂CH(OH)CH₂OH, and

CH₂triazole;

and all other variables are as previously defined.

In another embodiment of compounds of formula I, the compound is a diastereomer having the following structure:

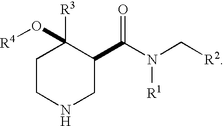

In another embodiment of compounds of formula I, the compound is an enantiomer having the following structure:

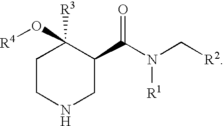

Specific examples of compounds of formula I, and pharmaceutically acceptable salts thereof, include those listed below:
rac-(3S,4R)—N-cyclopropyl-4-hydroxy-N-[3-(2-methoxyethoxy)-5-(3-methoxypropyl)benzyl]-4-phenylpiperidine-3-carboxamide,
rac-(3S,4R)—N-cyclopropyl-4-hydroxy-N-[3-(2-methoxyethoxy)-5-(3-methoxypropyl)benzyl]-4-pyridin-3-ylpiperidine-3-carboxamide,
rac-(3S,4R)—N-cyclopropyl-4-hydroxy-N-[3-(2-methoxyethoxy)-5-(3-methoxypropyl)benzyl]-4-pyridin-4-ylpiperidine-3-carboxamide,
rac-(3S,4R)—N-cyclopropyl-4-(4-fluorophenyl)-4-hydroxy-N-[3-(2-methoxyethoxy)-5-(3-methoxypropyl)benzyl]piperidine-3-carboxamide,
rac-(3S,4R)—N-cyclopropyl-4-(3-fluorophenyl)-4-hydroxy-N-[3-(2-methoxyethoxy)-5-(3-methoxypropyl)benzyl]piperidine-3-carboxamide,
rac-(3S,4R)—N-cyclopropyl-4-(3,4-difluorophenyl)-4-hydroxy-N-[3-(2-methoxyethoxy)-5-(3-methoxypropyl)benzyl]piperidine-3-carboxamide,
rac-(3S,4R)—N-cyclopropyl-4-(3,5-difluorophenyl)-4-hydroxy-N-[3-(2-methoxyethoxy)-5-(3-methoxypropyl)benzyl]piperidine-3-carboxamide,
rac-(3S,4R)-4-(3-chlorophenyl)-N-cyclopropyl-4-hydroxy-N-[3-(2-methoxyethoxy)-5-(3-methoxypropyl)benzyl]piperidine-3-carboxamide,
rac-(3S,4R)-4-(4-chlorophenyl)-N-cyclopropyl-4-hydroxy-N-[3-(2-methoxyethoxy)-5-(3-methoxypropyl)benzyl]piperidine-3-carboxamide,
rac-(3S,4R)-4-(4-chloro-3-fluorophenyl)-N-cyclopropyl-4-hydroxy-N-[3-(2-methoxyethoxy)-5-(3-methoxypropyl)benzyl]piperidine-3-carboxamide,
rac-(3S,4R)—N-cyclopropyl-4-(3,4-dichlorophenyl)-4-hydroxy-N-[3-(2-methoxyethoxy)-5-(3-methoxypropyl)benzyl]piperidine-3-carboxamide,
rac-(3S,4R)—N-cyclopropyl-4-hydroxy-N-[3-(2-methoxyethoxy)-5-(3-methoxypropyl)benzyl]-4-(2-methoxyphenyl)piperidine-3-carboxamide,
rac-(3S,4R)-4-[4-chloro-3-(trifluoromethyl)phenyl]-N-cyclopropyl-4-hydroxy-N-[3-(2-methoxyethoxy)-5-(3-methoxypropyl)benzyl]piperidine-3-carboxamide,
rac-(3S,4R)—N-cyclopropyl-4-[2-fluoro-4-(trifluoromethyl)phenyl]-4-hydroxy-N-[3-(2-methoxyethoxy)-5-(3-methoxypropyl)benzyl]piperidine-3-carboxamide,
rac-(3S,4R)—N-cyclopropyl-4-hydroxy-N-[3-(2-methoxyethoxy)-5-(3-methoxypropyl)benzyl]-4-(3-methoxyphenyl)piperidine-3-carboxamide,
rac-(3S,4R)-4-(3-aminophenyl)-N-cyclopropyl-4-hydroxy-N-[3-(2-methoxyethoxy)-5-(3-methoxypropyl)benzyl]piperidine-3-carboxamide,
rac-(3S,4R)—N-cyclopropyl-4-hydroxy-N-[3-(2-methoxyethoxy)-5-(3-methoxypropyl)benzyl]-4-(1,3-thiazol-2-yl)piperidine-3-carboxamide,
rac-(3S,4R)—N-cyclopropyl-4-hydroxy-N-[3-(2-methoxyethoxy)-5-(3-methoxypropyl)benzyl]-4-(1-methyl-1H-imidazol-2-yl)piperidine-3-carboxamide,
rac-(3S,4R)—N-cyclopropyl-4-hydroxy-N-[3-(2-methoxyethoxy)-5-(3-methoxypropyl)benzyl]-4-(1H-1,2,3-triazol-4-yl)piperidine-3-carboxamide,
rac-(3S,4R)—N-cyclopropyl-4-hydroxy-N-[3-(2-methoxyethoxy)-5-(3-methoxypropyl)benzyl]-4-(2-thienyl)piperidine-3-carboxamide,
rac-(3S,4R)-4-(1,3-benzoxazol-2-yl)-N-cyclopropyl-4-hydroxy-N-[3-(2-methoxyethoxy)-5-(3-methoxypropyl)benzyl]piperidine-3-carboxamide,
rac-(3S,4R)-4-[2-(benzyloxy)pyridin-4-yl]-N-cyclopropyl-4-hydroxy-N-[3-(2-methoxyethoxy)-5-(3-methoxypropyl)benzyl]piperidine-3-carboxamide,
rac-(3S,4R)—N-cyclopropyl-4-hydroxy-N-[3-(2-methoxyethoxy)-5-(3-methoxypropyl)benzyl]-4-(1-methyl-2-oxo-1,2-dihydropyridin-4-yl)piperidine-3-carboxamide,
rac-(3S,4R)—N-cyclopropyl-4-hydroxy-N-[3-(2-methoxyethoxy)-5-(3-methoxypropyl)benzyl]-4-(1-methyl-6-oxo-1,6-dihydropyridin-3-yl)piperidine-3-carboxamide,
rac-(3S,4R)—N-{[1,3-bis(3-methoxypropyl)-2,4-dioxo-1,2,3,4-tetrahydropyrimidin-5-yl]methyl}-N-cyclopropyl-4-(3,4-difluorophenyl)-4-hydroxypiperidine-3-carboxamide,
rac-(3S,4R)—N-[2-chloro-5-(2-methoxyethyl)benzyl]-N-cyclopropyl-4-(3,4-difluorophenyl)-4-hydroxypiperidine-3-carboxamide,
rac-(3S,4R)—N-cyclopropyl-N-(2,3-dichlorobenzyl)-4-(3,4-difluorophenyl)-4-hydroxypiperidine-3-carboxamide,
rac-(3S,4R)—N-[2-chloro-5-(3-methoxypropyl)benzyl]-N-cyclopropyl-4-(3,4-difluorophenyl)-4-hydroxypiperidine-3-carboxamide,
rac-(3S,4R)—N-{[5-chloro-2-(3-methoxypropyl)pyridin-4-yl]methyl}-N-cyclopropyl-4-(3,4-difluorophenyl)-4-hydroxypiperidine-3-carboxamide,
rac-(3S,4R)—N-{[5-chloro-2-(3-methoxypropyl)-1-oxidopyridin-4-yl]methyl}-N-cyclopropyl-4-(3,4-difluorophenyl)-4-hydroxypiperidine-3-carboxamide,
rac-(3S,4R)—N-(5-{[acetyl(methyl)amino]methyl}-2-chlorobenzyl)-N-cyclopropyl-4-(3,4-difluorophenyl)-4-hydroxypiperidine-3-carboxamide,
rac-(3S,4R)—N-cyclopropyl-4-(3,4-difluorophenyl)-4-hydroxy-N-{[1-(3-methoxypropyl)-1H-indol-3-yl]methyl}piperidine-3-carboxamide,
(3S,4R)—N-cyclopropyl-4-(3,4-difluorophenyl)-4-hydroxy-N-[3-(2-methoxyethoxy)-5-(3-methoxypropyl)benzyl]piperidine-3-carboxamide,
(3S,4R)—N-[2-chloro-5-(3-methoxypropyl)benzyl]-N-cyclopropyl-4-(3,4-difluorophenyl)-4-hydroxypiperidine-3-carboxamide,
Rac-(3S,4R)—N-cyclopropyl-4-(3,4-difluorophenyl)-4-methoxy-N-[3-(2-methoxyethoxy)-5-(3-methoxypropyl)benzyl]piperidine-3-carboxamide,
Rac-(3S,4R)—N-[2-chloro-5-(2-methoxyethyl)benzyl]-N-cyclopropyl-4-(3,4-difluorophenyl)-4-methoxypiperidine-3-carboxamide,
Rac-(3S,4R)—N-cyclopropyl-N-(2,3-dichlorobenzyl)-4-(3,4-difluorophenyl)-4-methoxypiperidine-3-carboxamide,
Rac-(3S,4R)—N-[2-chloro-5-(3-methoxypropyl)benzyl]-N-cyclopropyl-4-(3,4-difluorophenyl)-4-methoxypiperidine-3-carboxamide,
Rac-(3S,4R)—N-{[5-chloro-2-(3-methoxypropyl)pyridin-4-yl]methyl}-N-cyclopropyl-4-(3,4-difluorophenyl)-4-methoxypiperidine-3-carboxamide,
Rac-(3S,4R)—N-{[5-chloro-2-(3-methoxypropyl)-1-oxidopyridin-4-yl]methyl}-N-cyclopropyl-4-(3,4-difluorophenyl)-4-hydroxypiperidine-3-carboxamide,
Rac-(3S,4R)—N-cyclopropyl-4-(3,5-difluorophenyl)-4-methoxy-N-[3-(2-methoxyethoxy)-5-(3-methoxypropyl)benzyl]piperidine-3-carboxamide,
Rac-(3S,4R)—N-cyclopropyl-4-methoxy-N-[3-(2-methoxyethoxy)-5-(3-methoxypropyl)benzyl]-4-(1-methyl-2-oxo-1,2-dihydropyridin-4-yl)piperidine-3-carboxamide,
Rac-(3S,4R)—N-cyclopropyl-4-hydroxy-N-[3-(2-methoxyethoxy)-5-(3-methoxypropyl)benzyl]-4-(1-methyl-6-oxo-1,6-dihydropyridin-3-yl)piperidine-3-carboxamide,
Rac-(3S,4R)-4-(1-butyl-2-oxo-1,2-dihydropyridin-4-yl)-N-cyclopropyl-4-methoxy-N-[3-(2-methoxyethoxy)-5-(3-methoxypropyl)benzyl]piperidine-3-carboxamide,
Rac-(3S,4R)-4-(2-butoxypyridin-4-yl)-N-cyclopropyl-4-methoxy-N-[3-(2-methoxyethoxy)-5-(3-methoxypropyl)benzyl]piperidine-3-carboxamide,
(3S,4R)—N-cyclopropyl-4-(3,4-difluorophenyl)-4-methoxy-N-[3-(2-methoxyethoxy)-5-(3-methoxypropyl)benzyl]piperidine-3-carboxamide,
(3S,4R)—N-cyclopropyl-4-(3,4-difluorophenyl)-4-ethoxy-N-[3-(2-methoxyethoxy)-5-(3-methoxypropyl)benzyl]piperidine-3-carboxamide,
(3S,4R)—N-cyclopropyl-4-(3,4-difluorophenyl)-4-[(4-fluorobenzyl)oxy]-N-[3-(2-methoxyethoxy)-5-(3-methoxypropyl)benzyl]piperidine-3-carboxamide,
(3S,4R)—N-cyclopropyl-4-(3,4-difluorophenyl)-4-(2-methoxyethoxy)-N-[3-(2-methoxyethoxy)-5-(3-methoxypropyl)benzyl]piperidine-3-carboxamide,
(3S,4R)—N-cyclopropyl-4-(3,4-difluorophenyl)-4-(2,3-dihydroxypropoxy)-N-[3-(2-methoxyethoxy)-5-(3-methoxypropyl)benzyl]piperidine-3-carboxamide trifluoroacetate,
(3S,4R)—N-cyclopropyl-4-(3,4-difluorophenyl)-N-[3-(2-methoxyethoxy)-5-(3-methoxypropyl)benzyl]-4-(1H-1,2,3-triazol-5-ylmethoxy)piperidine-3-carboxamide,
(3S,4R)—N-cyclopropyl-4-methoxy-N-[3-(2-methoxyethoxy)-5-(3-methoxypropyl)benzyl]-4-(1-methyl-2-oxo-1,2-dihydropyridin-4-yl)piperidine-3-carboxamide,
(3S,4R)-4-(2-butoxypyridin-4-yl)-N-cyclopropyl-4-methoxy-N-[3-(2-methoxyethoxy)-5-(3-methoxypropyl)benzyl]piperidine-3-carboxamide, and
(3S,4R)—N-[2-chloro-5-(2-methoxyethyl)benzyl]-N-cyclopropyl-4-(3,4-difluorophenyl)-4-(2,3-dihydroxypropoxy)piperidine-3-carboxamide.

The compounds of Formula I above, and pharmaceutically acceptable salts thereof, are renin inhibitors. The compounds are useful for inhibiting renin and treating conditions such as hypertension. Any reference to a compound of formula (I) is to be understood as referring also to optically pure enantiomers, mixtures of enantiomers such as racemates, diastereomers, mixtures of diastereomers, diastereomeric racemates, mixtures of diastereomeric racemates, meso-forms and tautomers, as well as salts (especially pharmaceutically acceptable salts) and solvates (including hydrates) of such compounds, and morphological forms, as appropriate and expedient. The present invention encompasses all these forms. Mixtures are separated in a manner known per se, e.g. by column chromatography, thin layer chromatography (TLC), high performance liquid chromatography (HPLC), or crystallization. The compounds of the present invention may have chiral centers, e.g. one chiral center (providing for two stereoisomers, (R) and (S)), or two chiral centers (providing for up to four stereoisomers, (R,R), (S,S), (R,S), and (S,R)). This invention includes all of these optical isomers and mixtures thereof. Unless specifically mentioned otherwise, reference to one isomer applies to any of the possible isomers. Whenever the isomeric composition is unspecified, all possible isomers are included.

Tautomers of compounds defined in Formula I are also included within the scope of the present invention. For example, compounds including carbonyl —CH₂C(O)— groups (keto forms) may undergo tautomerism to form hydroxyl —CH═C(OH)— groups (enol forms). Both keto and enol forms are included within the scope of the present invention.

In addition, compounds with carbon-carbon double bonds may occur in Z- and E-forms with all isomeric forms of the compounds being included in the present invention.

Compounds of the invention also include nitrosated compounds of formula (I) that have been nitrosated through one or more sites such as oxygen (hydroxyl condensation), sulfur (sulfhydryl condensation) and/or nitrogen. The nitrosated compounds of the present invention can be prepared using conventional methods known to one skilled in the art. For example, known methods for nitrosating compounds are described in U.S. Pat. Nos. 5,380,758, 5,703,073, 5,994,294, 6,242,432 and 6,218,417; WO 98/19672; and Oae et al., Org. Prep. Proc. Int., 15(3): 165-198 (1983).

Salts are preferably the pharmaceutically acceptable salts of the compounds of formula (I). The expression “pharmaceutically acceptable salts” encompasses either salts with inorganic acids or organic acids like hydrochloric acid, hydrobromic acid, hydroiodic acid, sulfuric acid, sulfamic acid, phosphoric acid, nitric acid, phosphorous acid, nitrous acid, citric acid, formic acid, acetic acid, oxalic acid, maleic acid, lactic acid, tartaric acid, fumaric acid, benzoic acid, mandelic acid, cinnamic acid, palmoic acid, stearic acid, glutamic acid, aspartic acid, methanesulfonic acid, ethanesulfonic acid, ethanedisulfonic acid, p-toluenesulfonic acid, salicylic acid, succinic acid, trifluoroacetic acid, and the like that are non toxic to living organisms or, in case the compound of formula (I) is acidic in nature, with an inorganic base like an alkali or earth alkali base, e.g. sodium hydroxide, potassium hydroxide, calcium hydroxide and the like. For other examples of pharmaceutically acceptable salts, reference can be made notably to “Salt selection for basic drugs”, Int. J. Pharm. (1986), 33, 201-217.

The invention also includes derivatives of the compound of Formula I, acting as prodrugs. These prodrugs, following administration to the patient, are converted in the body by normal metabolic processes to the compound of Formula I. Such prodrugs include those that demonstrate enhanced bioavailability (see Table 4 below), tissue specificity, and/or cellular delivery, to improve drug absorption of the compound of Formula I. The effect of such prodrugs may result from modification of physicochemical properties such as lipophilicity, molecular weight, charge, and other physicochemical properties that determine the permeation properties of the drug.

The general terms used hereinbefore in formula I and hereinafter preferably have, within this disclosure, the following meanings, unless otherwise indicated. Where the plural form is used for compounds, salts, pharmaceutical compositions, diseases and the like, this is intended to mean also a single compound, salt, or the like.

The term “alkyl”, alone or in combination with other groups, unless indicated otherwise, means saturated, straight and branched chain groups with one to six carbon atoms (which may be represented by “C₁₋₆ alkyl” or “C₁-C₆ alkyl”). When the intended meaning is other than this, for example, when the number of carbon atoms is in the range of one to four carbon atoms, this meaning is represented in like fashion as “C₁₋₄ alkyl” or “C₁-C₄ alkyl”. Examples of alkyl groups are methyl, ethyl, n-propyl, iso-propyl, n-butyl, iso-butyl, sec-butyl, tert-butyl, pentyl, hexyl and heptyl. The methyl, ethyl and isopropyl groups are preferred. Structural depictions of compounds may show a terminal methyl group as “—CH₃”, “Me”, or

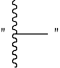

i.e., these have equivalent meanings.

The term “alkenyl”, alone or in combination with other groups, unless indicated otherwise, means unsaturated (i.e., having at least one double bond) straight and branched chain groups with two to six carbon atoms (which may be represented by “C₂₋₆ alkenyl” or “C₂-C₆ alkenyl”). When the intended meaning is other than this, for example, when the number of carbon atoms is in the range of two to four carbon atoms, this meaning is represented in like fashion as “C₂₋₄ alkenyl” or “C₂-C₄ alkenyl”.

The term “alkoxy”, alone or in combination with other groups, refers to an R—O— group, wherein R is an alkyl group. Examples of alkoxy groups are methoxy, ethoxy, propoxy, iso-propoxy, iso-butoxy, sec-butoxy and tert-butoxy.

The term “hydroxy-alkyl”, alone or in combination with other groups, refers to an HO—R— group, wherein R is an alkyl group. Examples of hydroxy-alkyl groups are HO—CH₂—, HO—CH₂CH₂—, HO—CH₂CH₂CH₂— and CH₃CH(OH)—.

The term “halogen” means fluorine, chlorine, bromine or iodine, preferably fluorine, chlorine or bromine, especially fluorine or chlorine.

The term “cycloalkyl”, alone or in combination with other groups, unless indicated otherwise, means a saturated cyclic hydrocarbon ring system with 3 to 8 carbon atoms, e.g., cyclopropyl, cyclobutyl, cyclopentyl, cyclohexyl, cycloheptyl and cyclooctyl. This may be represented by “C₃₋₈ cycloalkyl” or “C₃-C₈ cycloalkyl”). When the intended meaning is other than this, for example, when the number of carbon atoms is in the range of three to six carbon atoms, this meaning is represented in like fashion as “C₃₋₆ cycloalkyl” or “C₃-C₆ cycloalkyl”.

The term “aryl”, alone or in combination, relates to a phenyl, naphthyl or indanyl group, preferably a phenyl group. The abbreviation “Ph” represents phenyl.

The term “heteroaryl”, alone or in combination, means a 5 or 6-membered aromatic ring containing 1, 2, 3 or 4 heteroatoms independently selected from N, O or S aromatic rings, e.g., 5-membered rings containing one nitrogen (pyrrole), one oxygen (pyran) or one sulfur (thiophene) atom, 5-membered rings containing one nitrogen and one sulfur (thiazole) atom, 5-membered rings containing one nitrogen and one oxygen (oxazole or isoxazole) atom, 5-membered rings containing two nitrogen (imidazole or pyrazole) atoms, five-membered aromatic rings containing three nitrogen atoms, five-membered aromatic rings containing one oxygen, one nitrogen or one sulfur atom, five-membered aromatic rings containing two heteroatoms independently selected from oxygen, nitrogen and sulfur, 6-membered rings containing one nitrogen (pyridine), or one oxygen (furan) atom, 6-membered rings containing two nitrogen (pyrazine, pyrimidine, or pyridazine) atoms, 6-membered rings containing three nitrogen (triazine) atoms, a tetrazolyl ring; a thiazinyl ring; or coumarinyl. Examples of such ring systems are furanyl, thienyl, pyrrolyl, pyridinyl, pyrimidinyl, indolyl, imidazolyl, triazinyl, thiazolyl, isothiazolyl, pyridazinyl, pyrazolyl, oxazolyl, and isoxazolyl.

The term “fused heteroaryl”, alone or in combination, means a 9 or 10-membered aromatic ring system containing 1, 2, 3 or 4 heteroatoms independently selected from N, O and S, fused to a benzene ring, e.g., benzofused six-membered aromatic rings containing one to three nitrogen atoms, and benzofused five-membered aromatic rings containing one oxygen, one nitrogen or one sulfur atom. Examples of such ring systems are benzothienyl, benzoxazole, benzimidazole, quinolinyl, isoquinolinyl, quinazolinyl and quinoxalinyl.

The present invention also encompasses a pharmaceutical formulation comprising a pharmaceutically acceptable carrier and the compound of Formula I or a pharmaceutically acceptable crystal form or hydrate thereof. A preferred embodiment is a pharmaceutical composition of the compound of Formula I, comprising, in addition, a second agent.

### LIST OF ABBREVIATIONS

ABTS 2,2′-Azino-bis(3-ethylbenzthiazoline-6-sulfonic Acid).2NH₃
Ag₂CO₃ silver carbonate
BOC (Boc) t-butyloxycarbonyl
BOC₂O di-tert-butyl dicarbonate
n-BuLi n-butyllithium
BSA bovine serum albumin
CBr₄ carbone tetrabromide
CH₂Cl₂ dichloromethane
CuI copper iodide
DBU 1,8-diazabicyclo[5.4.0]undec-7-ene
DIBAH diisobutylaluminum hydride
DME 1,2-dimethoxyethane
DMF dimethylformamide
DMSO dimethylsulfoxide
EDTA ethylenediaminetetraacetic acid
EIA enzyme immunoassay
EtOAc ethyl acetate
Et₂O diethylether
Et₃N triethylamine
HATU O-(7-azabenzotriazol-1-yl)-N,N,N′,N′-tetramethyluronium hexafluorophosphate
HCl hydrochloric acid
Hex hexane
HMPA hexamethylphosphoramide
HPLC high pressure liquid chromatography
K₂CO₃ potassium carbonate
K₃Fe(CN)₆ potassium ferricyanide
LiHMDS lithium hexamethyldisilazide
MeCN acetonitrile
MeI iodomethane
MeOH methanol
MgBr₂ magnesium bromide
MgSO₄ magnesium sulfate
NaI sodium iodide
NaOH sodium hydroxide
NBS N-bromo succinimide
NaBH₄ sodium borohydride
NaHCO₃ sodium bicarbonate
Na₂CO₃ sodium carbonate
Na₂SO₄ sodium sulfate
NH₄Cl ammonium chloride
NMR nuclear magnetic resonance
PBS phosphate-buffered saline
iPrOH isopropanol
PPh₃ triphenylphosphine
RT (rt) room temperature
SiO₂ silicon dioxide
S—PHOS dicyclohexylphosphino-2′-6′-dimethoxy-1-1′-biphenyl
TBS tert-butyldimethylsilyl
TBSO tert-butyldimethylsilyloxy
TEA triethylamine
TFA trifluoroacetic acid
THF tetrahydrofuran
TLC thin layer chromatography
Tol toluene

Unless expressly stated to the contrary, all ranges cited herein are inclusive. For example, an alkyl group described as C₁-C₆ alkyl means the alkyl group can contain 1, 2, 3, 4, 5 or 6 carbon atoms.

When any variable occurs more than one time in any constituent or in any formula depicting and describing compounds of the invention, its definition on each occurrence is independent of its definition at every other occurrence. Also, combinations of substituents and/or variables are permissible only if such combinations result in stable compounds.

The term “substituted” (e.g., as in “aryl which is optionally substituted with one or more substituents . . . ”) includes mono- and poly-substitution by a named substituent to the extent such single and multiple substitution (including multiple substitution at the same site) is chemically allowed.

In compounds of the invention having pyridyl N-oxide moieties, the pyridyl-N-oxide portion is structurally depicted using conventional representations such as

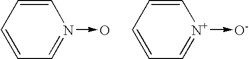

which have equivalent meanings.

The invention relates to a method for the treatment and/or prophylaxis of diseases which are related to hypertension, congestive heart failure, pulmonary hypertension, systolic hypertension, renal insufficiency, renal ischemia, renal failure, renal fibrosis, cardiac insufficiency, cardiac hypertrophy, cardiac fibrosis, myocardial ischemia, cardiomyopathy, glomerulonephritis, renal colic, complications resulting from diabetes such as nephropathy, vasculopathy and neuropathy, glaucoma, elevated intra-ocular pressure, atherosclerosis, restenosis post angioplasty, complications following vascular or cardiac surgery, erectile dysfunction, hyperaldosteronism, lung fibrosis, scleroderma, anxiety, cognitive disorders, complications of treatments with immunosuppressive agents, and other diseases known to be related to the renin-angiotensin system, which method comprises administrating a compound as defined above to a human being or animal.

In another embodiment, the invention relates to a method for the treatment and/or prophylaxis of diseases which are related to hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, complications resulting from diabetes such as nephropathy, vasculopathy and neuropathy.

In another embodiment, the invention relates to a method for the treatment and/or prophylaxis of diseases, which are associated with a dysregulation of the renin-angiotensin system as well as for the treatment of the above-mentioned diseases.

The invention also relates to the use of compounds of formula (I) for the preparation of a medicament for the treatment and/or prophylaxis of the above-mentioned diseases.

Compounds of formula (I) or the above-mentioned pharmaceutical compositions are also of use in combination with other pharmacologically active compounds comprising ACE-inhibitors, neutral endopeptidase inhibitors, angiotensin II receptor antagonists, endothelin receptors antagonists, vasodilators, calcium antagonists, potassium activators, diuretics, sympatholitics, beta-adrenergic antagonists, alpha-adrenergic antagonists or with other drugs beneficial for the prevention or the treatment of the above-mentioned diseases.

The term “administration” and variants thereof (e.g., “administering” a compound) in reference to a compound of Formula I mean providing the compound or a prodrug of the compound to the individual in need of treatment or prophylaxis. When a compound of the invention or a prodrug thereof is provided in combination with one or more other active agents (e.g., an agent such as anangiotensin II receptor antagonist, ACE inhibitor, or other active agent which is known to reduce blood pressure), “administration” and its variants are each understood to include provision of the compound or prodrug and other agents at the same time or at different times. When the agents of a combination are administered at the same time, they can be administered together in a single composition or they can be administered separately.

As used herein, the term “composition” is intended to encompass a product comprising the specified ingredients in the specified amounts, as well as any product which results, directly or indirectly, from combining the specified ingredients in the specified amounts.

By “pharmaceutically acceptable” is meant that the ingredients of the pharmaceutical composition must be compatible with each other and not deleterious to the recipient thereof.

The term “subject” as used herein refers to an animal, preferably a mammal, most preferably a human, who has been the object of treatment, observation or experiment.

The term “effective amount” as used herein means that amount of active compound or pharmaceutical agent that elicits the biological or medicinal response in a tissue, system, animal or human that is being sought by a researcher, veterinarian, medical doctor or other clinician. In one embodiment, the effective amount is a “therapeutically effective amount” for the alleviation of the symptoms of the disease or condition being treated. In another embodiment, the effective amount is a “prophylactically effective amount” for prophylaxis of the symptoms of the disease or condition being prevented. The term also includes herein the amount of active compound sufficient to inhibit renin and thereby elicit the response being sought (i.e., an “inhibition effective amount”). When the active compound (i.e., active ingredient) is administered as the salt, references to the amount of active ingredient are to the free form (i.e., the non-salt form) of the compound.

In a preferred embodiment, this amount is comprised between 1 mg and 1000 mg per day. In a particularly preferred embodiment, this amount is comprised between 1 mg and 500 mg per day. In a more particularly preferred embodiment, this amount is comprised between 1 mg and 200 mg per day.

In the method of the present invention (i.e., inhibiting renin), the compounds of Formula I, optionally in the form of a salt, can be administered by any means that produces contact of the active agent with the agent's site of action. They can be administered by any conventional means available for use in conjunction with pharmaceuticals, either as individual therapeutic agents or in a combination of therapeutic agents. They can be administered alone, but typically are administered with a pharmaceutical carrier selected on the basis of the chosen route of administration and standard pharmaceutical practice. The compounds of the invention can, for example, be administered orally, parenterally (including subcutaneous injections, intravenous, intramuscular, intrasternal injection or infusion techniques), by inhalation spray, or rectally, in the form of a unit dosage of a pharmaceutical composition containing an effective amount of the compound and conventional non-toxic pharmaceutically-acceptable carriers, adjuvants and vehicles. Liquid preparations suitable for oral administration (e.g., suspensions, syrups, elixirs and the like) can be prepared according to techniques known in the art and can employ any of the usual media such as water, glycols, oils, alcohols and the like. Solid preparations suitable for oral administration (e.g., powders, pills, capsules and tablets) can be prepared according to techniques known in the art and can employ such solid excipients as starches, sugars, kaolin, lubricants, binders, disintegrating agents and the like. Parenteral compositions can be prepared according to techniques known in the art and typically employ sterile water as a carrier and optionally other ingredients, such as a solubility aid. Injectable solutions can be prepared according to methods known in the art wherein the carrier comprises a saline solution, a glucose solution or a solution containing a mixture of saline and glucose. Further description of methods suitable for use in preparing pharmaceutical compositions for use in the present invention and of ingredients suitable for use in said compositions is provided in Remington's Pharmaceutical Sciences, 18^{th} edition, edited by A. R. Gennaro, Mack Publishing Co., 1990.

### Methods of Synthesis

Compounds of the present invention can be made by a variety of methods depicted in the illustrative synthetic reaction schemes shown and described below. The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, Volumes 1-21; R. C. LaRock, Comprehensive Organic Transformations, 2.sup.nd edition Wiley-VCH, New York 1999; Comprehensive Organic Synthesis, B. Trost and I. Fleming (Eds.) vol. 1-9 Pergamon, Oxford, 1991; Comprehensive Heterocyclic Chemistry, A. R. Katritzky and C. W. Rees (Eds) Pergamon, Oxford 1984, vol. 1-9; Comprehensive Heterocyclic Chemistry II, A. R. Katritzky and C. W. Rees (Eds) Pergamon, Oxford 1996, vol. 1-11; and Organic Reactions, Wiley & Sons: New York, 1991, Volumes 1-40. The following synthetic reaction schemes and examples are merely illustrative of some methods by which the compounds of the present invention can be synthesized, and various modifications to these synthetic reaction schemes can be made and will be suggested to one skilled in the art having referred to the disclosure contained in this application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data. Unless specifically stated otherwise, the experimental procedures were performed under the following conditions. Evaporation of solvent was carried out using a rotary evaporator under reduced pressure (600-4000 pascals: 4.5-30 mm Hg) with a bath temperature of up to 60° C. Reactions are typically run under nitrogen atmosphere at ambient temperature if not otherwise mentioned. Anhydrous solvent such as THF, DMF, Et₂O, DME and Toluene are commercial grade. Reagents are commercial grade and were used without further purification. Flash chromatography is run on silica gel (230-400 mesh). The course of the reaction was followed by either thin layer chromatography (TLC) or nuclear magnetic resonance (NMR) spectrometry and reaction times given are for illustration only. The structure and purity of all final products were ascertained by TLC, mass spectrometry, ¹H NMR and/or high-pressure liquid chromatography (HPLC). Chemical symbols have their usual meanings. The following abbreviations have also been used: v (volume), w (weight), b.p. (boiling point), m.p. (melting point), L (liter(s)), mL (milliliter(s)), g (gram(s)), mg (milligram(s)), mol (mole(s)), mmol (millimole(s)), eq. (equivalent(s)). Unless otherwise specified, all variables mentioned below have the meanings as provided above.

Compounds of the present invention can be prepared according to the following general procedure depicted in Scheme 1. For example, thermal condensation of the known N—BOC-protected ketoester 1 (Tetrahedron: Asymmetry, 15(20), 3281-3287; 2004; European Journal of Organic Chemistry, (4), 721-726; 2003; Journal of the Chemical Society, Perkin Transactions 1: Organic and Bio-Organic Chemistry, (22), 3673-3684; 1998) with a secondary amine of general structure 2 can provide the corresponding ketoamide 3. Addition of an aryl or heteroaryl magnesium halide reagent (commercially available or prepared as described below) to the ketoamide 3 in the presence or absence of LiCl provides a mixture of easily separable diastereomeric tertiary alcohols, from which the desired isomer 4 is isolated. At this stage, the R₂ group of 4 may or may not be derivatized by subsequent chemistry. The tertiary alcohol 4 can then be deprotected using standard N—BOC deprotecting conditions (HCl or ZnBr₂) to afford the piperidine 5. Subsequent to deprotection, the tertiary alcohol may also be resolved to afford the active enantiomer 6. The tertiary alcohol 4 may be derivatized with alkyl groups and deprotected to afford racemic tertiary alkyl ether 7. Enantiopure tertiary alcohol 4 may be obtained either by reprotection of piperidine 6 or directly by resolution of racemic 4. The enantiopure tertiary alcohol 4 can be derivatized with alkyl groups and deprotected to afford chiral tertiary alkyl ether 8.

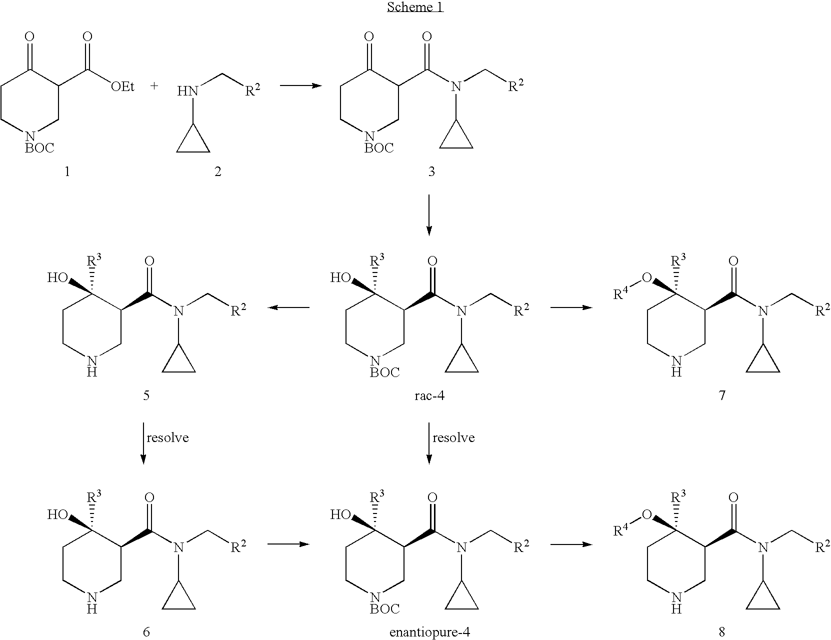

The amines 2 can be prepared as described below.

AMINE 2

| Compound | Structure |
|---|---|
| 2.1 | 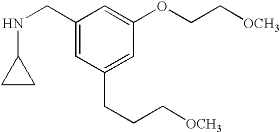 |
| 2.2 | 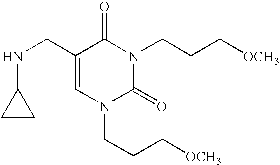 |
| 2.3 | 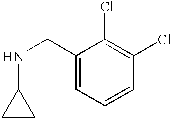 |
| 2.4 | 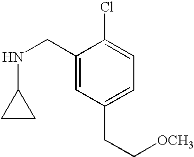 |
| 2.5 | 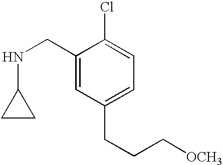 |
| 2.6 | 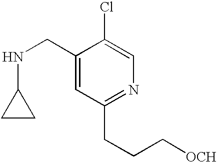 |
| 2.7 | 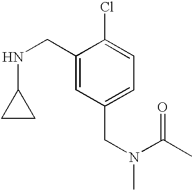 |
| 2.8 | 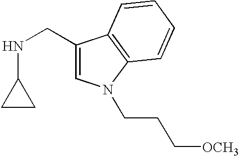 |

### Amine 2.1; N-[3-(2-methoxyethoxy)-5-(3-methoxypropyl)benzyl]cyclopropanamine

### Step 1: 1,3-dibromo-5-(2-methoxyethoxy)benzene

To a mixture of 3,5-dibromophenol (1 eq.) and 1-bromo-3-methoxypropane (1.5 eq.) in THF:DMF (4:1, 0.19 M) was added cesium carbonate (1.3 eq.). The mixture was heated to 80° C. for 3 h. The reaction was then cooled to RT and diluted with ethyl acetate and the organic phase was washed with NH₄Cl, water, followed by brine. The organic phase was then separated, dried (MgSO₄), filtered and concentrated in vacuo. The crude mixture was then purified by flash chromatography over silica gel (10% EtOAc in hexanes) to afford the title compound.

### Step 2: 3-bromo-5-(2-methoxyethoxy)benzaldehyde

To a solution (0.8 M) of n-butyl lithium (2.5 M in hexanes, 0.8 eq.) in toluene (0.8 M) at −15° C. was added dropwise over 5 minutes n-butyl magnesium chloride (2.0 M in THF, 0.4 eq.). The reaction mixture was stirred at −15° C. for 20 min before a toluene solution (0.3 M) of 1,3-dibromo-5-(2-methoxyethoxy)benzene from step 1 (1 eq.) was added dropwise at −15° C. over a period of 45 min. The resulting suspension was then warmed to 0° C. and stiffed for 1.5 h. The reaction was then cooled back down to −10° C. before DMF (3 eq.) was added dropwise. The reaction mixture was then slowly warmed to 0° C. and allowed to stir at 0° C. for 30 min. The reaction was then cooled to −5° C. and stirred for an additional 0/N at this temperature. The reaction was then carefully quenched with potassium acetate (5 eq.) and then diluted with ether. The organic phase was washed twice with water then brine, dried (MgSO₄), filtered and concentrated in vacuo. Purification of the crude residue by flash chromatography over silica gel (5% EtOAc in toluene) afforded the title compound.

### Step 3: 3-(2-methoxyethoxy)-5-(3-methoxypropyl)benzaldehyde

Allyl methyl ether (2.16 eq.) was added directly to 9-borabicyclo[3.3.1]nonane (0.5 M in THF, 1.9 eq.) at RT and stirred for 1 h. The mixture was then warmed to 70° C. for 5 min to which was added a degassed mixture (mixture degassed by way of purging with N₂ for 5 min) of 1,1′-bis(diphenylphosphino)ferrocene-palladium(II) dichloride dichloromethane complex (3 mol %), 3-bromo-5-(2-methoxyethoxy)benzaldehyde from step 2 (1 eq.) and tripotassium phosphate (2.5 eq.) in DMF (0.3 M). The reaction mixture was stiffed at 70° C., O/N then cooled to RT. The crude reaction mixture was then diluted with Et₂O and organic phase washed twice with water, dried (MgSO₄), filtered, and concentrated in vacuo. Purification of the crude residue by flash chromatography over silica gel (25-35% EtOAc in toluene) afforded the title compound.

### Step 4: N-[3-(2-methoxyethoxy)-5-(3-methoxypropyl)benzyl]cyclopropanamine

To a solution of 3-(2-methoxyethoxy)-5-(3-methoxypropyl)benzaldehyde from step 3 (1 eq.) in dichloromethane (0.5 M) was added cyclopropylamine (2 eq.) followed by magnesium sulfate (2.7 eq.). The resulting suspension was stirred 0/N at RT. The insolubles were removed via filtration. Concentration of the filtrate in vacuo afforded the crude imine. The imine was then taken up in methanol (0.3 M) and cooled to 0° C. to which was added potassium acetate (2.5 eq.) followed by sodium cyanoborohydride (1.2 eq.). The reaction mixture was then stirred at 0° C. for 20 min then RT for 10 min. The reaction mixture was then inversely quenched by pouring it into cold aqueous NaHCO₃ and the crude extracted three times with EtOAc. The combined organic extracts were dried over MgSO₄, filtered and concentrated in vacuo to afford the title compound.

Amine 2.2; 5-[(cyclopropylamino)methyl]-1,3-bis(3-methoxypropyl)pyrimidine-2,4(1H,3H)-dione

### Step 1: 1,3-bis(3-methoxypropyl)-2,4-dioxo-1,2,3,4-tetrahydropyrimidine-5-carbaldehyde

To a solution of 2,4-dioxo-1,2,3,4-tetrahydropyrimidine-5-carbaldehyde (1 eq.) and 1-bromo-3-methoxypropane (2.2 eq.) at rt in DMF (0.36 M) was added DBU (2.2 eq.). The reaction mixture was stirred at rt 3 days, concentrated in vacuo, and the residue purified by flash chromatography (SiO₂; EtOAc) to afford the title compound.

### Step 2: 5-[(cyclopropylamino)methyl]-1,3-bis(3-methoxypropyl)pyrimidine-2,4(1H,3H)-dione

To a solution of the title compound from step 1 (1 eq.) and cyclopropylamine (1.1 eq.) in CH₂Cl₂ (0.1 M) at rt was added MgSO₄ (1 eq.), and the resulting mixture stirred at rt for 16 h, filtered and concentrated. The residue was taken up in MeOH (0.1 M) and cooled to 0° C. NaBH₄ (1.1 eq.) was added and the resulting mixture allowed to warm to room temperature over 16 h. The mixture was cooled, quenched with NaHCO₃ (aq. sat.) and diluted with EtOAc. The organic phase was washed with NaHCO₃ (aq. sat.), brine, dried over Na₂SO₄, filtered, and concentrated in vacuo. The residue was purified by flash chromatography (SiO₂; 5-10% (2M NH₃ in MeOH) in CH₂Cl₂) to afford the title compound as a colorless oil.

### Amine 2.3; N-(2,3-dichlorobenzyl)cyclopropanamine

The title compound was prepared according to the procedure described in the patent WO 2007/009250 A1.

### Amine 2.4; N-[2-chloro-5-(2-methoxyethyl)benzyl]cyclopropanamine

The title compound was prepared according to the procedure described in the patent WO 2007/009250 A1.

### Amine 2.5; N-[2-chloro-5-(3-methoxypropyl)benzyl]cyclopropanamine

The title compound was prepared according to the procedure described in the patent WO 2007/009250 A1.

### Amine 2.6; N-{[5-chloro-2-(3-methoxypropyl)pyridin-4-yl]methyl}cyclopropanamine

The title compound was prepared according to the procedure described in the patent WO 2007/009250 A1.

### Amine 2.7; N-{4-chloro-3-[(cyclopropylamino)methyl]benzyl}-N-methylacetamide

The title compound was prepared according to the procedure described in the patent WO 2007/009250 A1.

### Amine 2.8; N-{[1-(3-methoxypropyl)-1H-indol-3-yl]methyl}cyclopropanamine

The title compound was prepared according to the procedure described in the patent WO 2006/125621 A1.

The ketoamides 3 can be prepared as described below.

KETO AMIDE 3

| Com- | |
|---|---|
| pound | Structure |
| 3.1 | 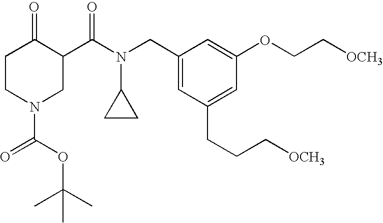 |
| 3.2 | 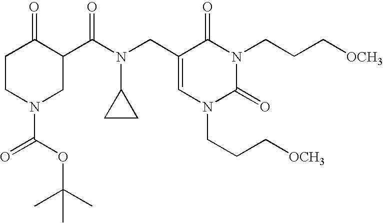 |
| 3.3 | 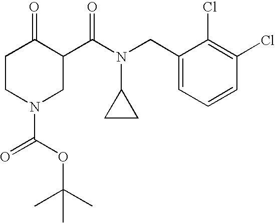 |
| 3.4 | 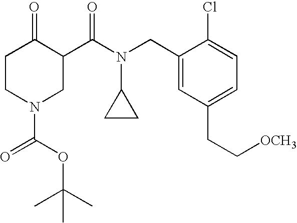 |
| 3.5 | 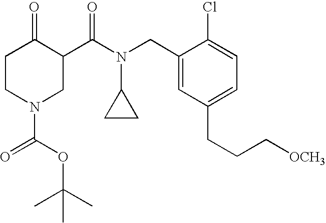 |
| 3.6 | 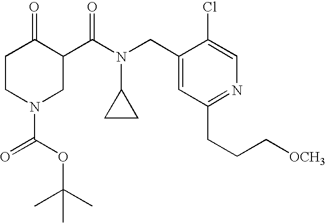 |
| 3.7 | 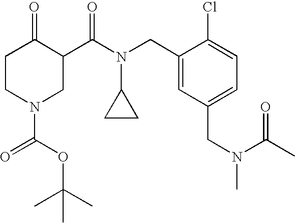 |
| 3.8 | 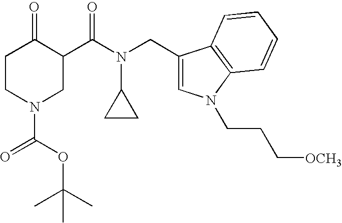 |

### Ketoamide 3.1; tert-butyl 3-({cyclopropyl[3-(2-methoxyethoxy)-5-(3-methoxypropyl)benzyl]amino}carbonyl)-4-oxopiperidine-1-carboxylate

1-tert-Butyl 3-ethyl 4-oxopiperidine-1,3-dicarboxylate 1 (1.1 eq.), amine 2.1 (1.0 eq.) and dimethylaminopyridine (0.2 eq.) were combined in a round bottom flask and heated to 14° C. under N2 until crude ¹H NMR revealed the reaction to be complete. Cooled slightly and added toluene. Purification by automated flash chromatography on silica gel (15-100% EtOAc in hexanes) afforded the title compound as an off-white solid.

### Ketoamide 3.2; tert-butyl 3-{[{[1,3-bis(3-methoxypropyl)-2,4-dioxo-1,2,3,4-tetrahydropyrimidin-5-yl]methyl}(cyclopropyl)amino]carbonyl}-4-oxopiperidine-1-carboxylate

The title compound is prepared analogously as described for the title compound 3.1 using amine 2.2. Purification by flash chromatography on silica gel (3-5% MeOH in CH₂Cl₂) afforded the title compound as a yellow oil.

### Ketoamide 3.3; tert-butyl 3-{[cyclopropyl(2,3-dichlorobenzyl)amino]carbonyl}-4-oxopiperidine-1-carboxylate

The title compound is prepared analogously as described for the title compound 3.1 using amine 2.3. Purification by automated flash chromatography on silica gel (20-60% EtOAc in hexanes) afforded the title compound.

### Ketoamide 3.4; tert-butyl 3-{[[2-chloro-5-(2-methoxyethyl)benzyl](cyclopropyl)amino]carbonyl}-4-oxopiperidine-1-carboxylate

The title compound is prepared analogously as described for the title compound 3.1 using amine 2.4. Purification by automated flash chromatography on silica gel (20-100% EtOAc in hexanes) afforded the title compound.

### Ketoamide 3.5; tert-butyl 3-{[[2-chloro-5-(3-methoxypropyl)benzyl](cyclopropyl)amino]carbonyl}-4-oxopiperidine-1-carboxylate

The title compound is prepared analogously as described for the title compound 3.1 using amine 2.5. Purification by automated flash chromatography on silica gel (20-100% EtOAc in hexanes) afforded the title compound.

### Ketoamide 3.6; tert-butyl 3-{[{[5-chloro-2-(3-methoxypropyl)pyridin-4-yl]methyl}(cyclopropyl)amino]carbonyl}-4-oxopiperidine-1-carboxylate

The title compound is prepared analogously as described for the title compound 3.1 using amine 2.6. Purification by automated flash chromatography on silica gel (0-75% EtOAc in hexanes) afforded the title compound as a yellow solid.

### Ketoamide 3.7; tert-butyl 3-{[(5-{[acetyl(methyl)amino]methyl}-2-chlorobenzyl)(cyclopropyl)amino]carbonyl}-4-oxopiperidine-1-carboxylate

The title compound is prepared analogously as described for the title compound 3.1 using amine 2.7. Purification by automated flash chromatography on silica gel (0-10% MeOH in EtOAc) afforded the title compound as a yellow foam.

### Ketoamide 3.8; tert-butyl 3-[(cyclopropyl{[1-(3-methoxypropyl)-1H-indol-3-yl]methyl}amino)carbonyl]-4-oxopiperidine-1-carboxylate

The title compound is prepared analogously as described for the title compound 3.1 using amine 2.8. Purification by automated flash chromatography on silica gel (30-100% EtOAc in hexanes) afforded the title compound.

Examples are shown below, along with renin IC₅₀ (nM) data for representative examples according to results obtains using FRET (quenched fluorescence resonance energy transfer) and human plasma assays.

### EXAMPLES

RACEMIC TERTIARY ALCOHOL 5

| Example | Compound | R² | R³ |
|---|---|---|---|
| 1 QFRET-318 Plasma-990 | 5.1 | 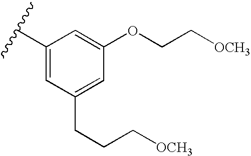 | 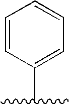 |
| 2 QFRET-140 Plasma-280 | 5.2 | 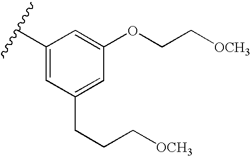 | 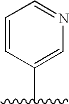 |
| 3 | 5.3 | 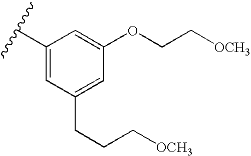 | 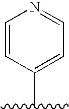 |
| 4 | 5.4 | 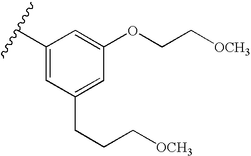 | 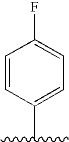 |
| 5 | 5.5 | 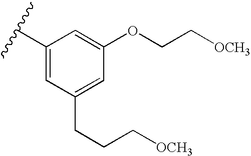 | 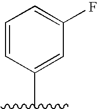 |
| 6 QFRET-5.7 Plasma-38 | 5.6 | 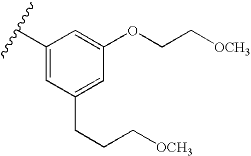 | 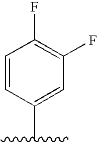 |
| 7 QFRET-23 Plasma-230 | 5.7 | 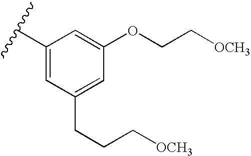 | 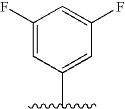 |
| 8 | 5.8 | 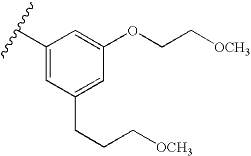 | 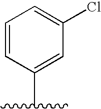 |
| 9 | 5.9 | 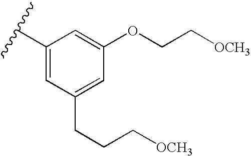 | 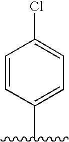 |
| 10 | 5.10 | 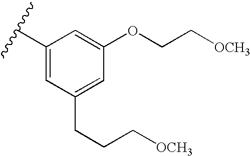 | 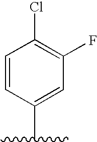 |
| 11 | 5.11 | 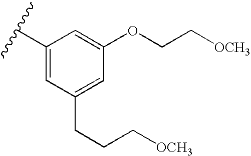 | 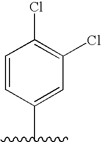 |
| 12 | 5.12 | 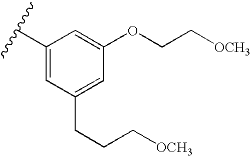 | 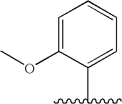 |
| 13 | 5.13 | 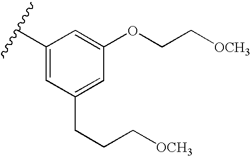 | 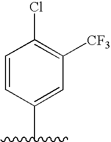 |
| 14 | 5.14 | 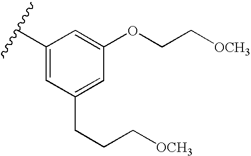 | 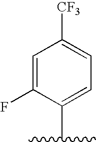 |
| 15 | 5.15 | 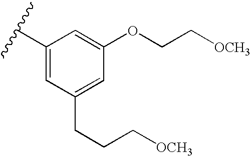 | 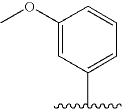 |
| 16 | 5.16 | 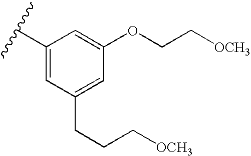 | 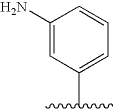 |
| 17 | 5.17 | 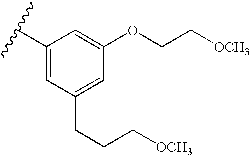 | 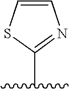 |
| 18 | 5.18 | 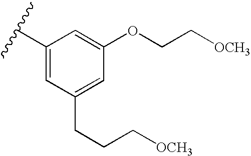 | 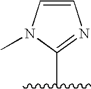 |
| 19 | 5.19 | 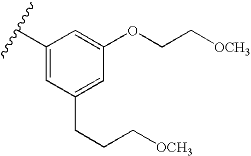 | 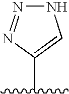 |
| 20 | 5.20 | 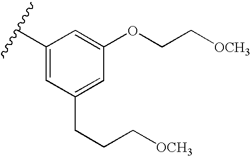 | 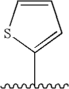 |
| 21 | 5.21 | 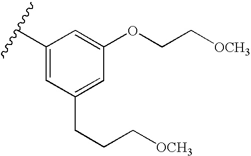 | 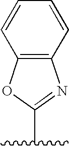 |
| 22 | 5.22 | 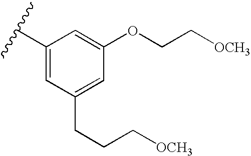 | 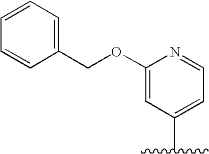 |
| 23 QFRET-76 Plasma-136 | 5.23 | 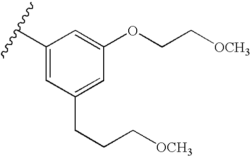 | 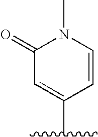 |
| 24 | 5.24 | 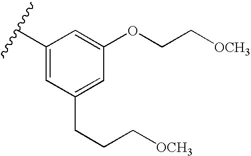 | 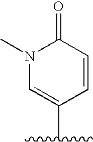 |
| 25 | 5.25 | 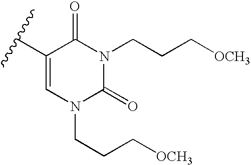 | 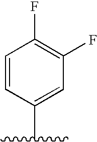 |
| 26 | 5.26 | 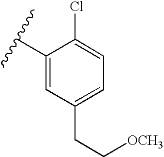 | 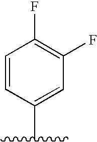 |
| 27 | 5.27 | 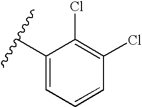 | 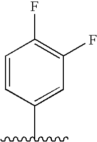 |
| 28 | 5.28 | 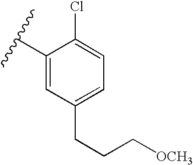 | 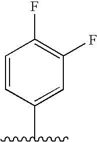 |
| 29 | 5.29 | 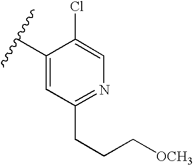 | 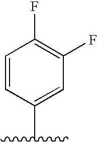 |
| 30 | 5.30 | 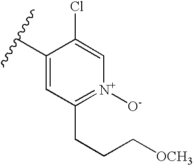 | 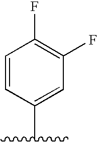 |
| 31 | 5.31 | 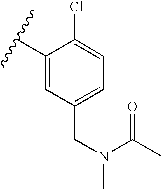 | 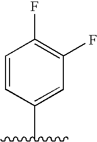 |
| 32 | 5.32 | 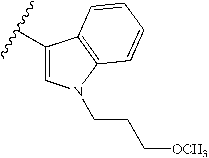 | 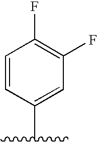 |

### Example 1

### rac-(3S,4R)—N-cyclopropyl-4-hydroxy-N-[3-(2-methoxyethoxy)-5-(3-methoxypropyl)benzyl]-4-phenylpiperidine-3-carboxamide

### Step 1: rac-tert-butyl (3S,4R)-3-({cyclopropyl[3-(2-methoxyethoxy)-5-(3-methoxypropyl)benzyl]amino}carbonyl)-4-hydroxy-4-phenylpiperidine-1-carboxylate

To a solution of ketoamide 3.1 (1.0 eq.) in THF (0.1 M) at RT under N₂ was added commercially available phenylmagnesium chloride (2M in THF, 2.07 eq.). The reaction was stirred at rt for 15 min. The reaction was then quenched with NH₄Cl and extracted 3× with EtOAc. The combined organic extracts were dried (MgSO₄), filtered and concentrated in vacuo. Purification by automated flash chromatography on silica gel (10-60% EtOAc in hexanes) afforded the title compound as a clear colorless oil.

### Step 2: rac-(3S,4R)—N-cyclopropyl-4-hydroxy-N-[3-(2-methoxyethoxy)-5-(3-methoxypropyl)benzyl]-4-phenylpiperidine-3-carboxamide

To a solution of the title compound from step 1 (1 eq.) in CH₂Cl₂ (0.1 M) was added HCl (4 M in dioxane, 36 eq.). The reaction was stirred at rt for 45 min. The reaction was then concentrated in vacuo. Purification by automated flash chromatography on silica gel (5% 2M NH₃ in MeOH/95% CH₂Cl₂) afforded the title compound as a clear colorless oil. MS: ESI+ve 497.4 (MH+).

### Example 2

### rac-(3S,4R)—N-cyclopropyl-4-hydroxy-N-[3-(2-methoxyethoxy)-5-(3-methoxypropyl)benzyl]-4-pyridin-3-ylpiperidine-3-carboxamide

### Step 1: rac-tert-butyl (3S,4R)-3-({cyclopropyl[3-(2-methoxyethoxy)-5-(3-methoxypropyl)benzyl]amino}carbonyl)-4-hydroxy-4-pyridin-3-ylpiperidine-1-carboxylate

3-Pyridylmagnesium bromide was prepared as follows: to a solution of 3-bromopyridine (1 eq.) in THF (0.147 M) at rt was added isopropylmagnesium chloride (1 eq.). The reaction mixture was stirred at rt for 30 min, affording a 0.1 M solution of 3-pyridylmagnesium bromide, which must be used immediately. To a solution of keto amide 3.1 (1.0 eq.) in THF (0.1 M) at rt under N₂ was added 3-pyridylmagnesium bromide (0.1 M in THF, 1.56 eq.). The reaction was stirred at rt for 5 min. The reaction was then quenched with NH₄Cl and extracted 3× with EtOAc. The combined organic extracts were dried (MgSO₄), filtered and concentrated in vacuo. Purification by automated flash chromatography on silica gel (30-100% EtOAc in hexanes) afforded the title compound.

### Step 2: rac-(3S,4R)—N-cyclopropyl-4-hydroxy-N-[3-(2-methoxyethoxy)-5-(3-methoxypropyl)benzyl]-4-pyridin-3-ylpiperidine-3-carboxamide

To a solution of the title compound from step 1 (1 eq.) in CH₂Cl₂ (0.02 M) at rt was added HCl (4 M in dioxane, 36 eq.). The reaction was stirred at rt for 1 h. The reaction was then concentrated in vacuo. Purification by column chromatography on silica gel (10% 2M NH₃ in MeOH/90% CH₂Cl₂) afforded the title compound. MS: 498.5 ESI+ve (MH+).

### Example 3

### rac-(3S,4R)—N-cyclopropyl-4-hydroxy-N-[3-(2-methoxyethoxy)-5-(3-methoxypropyl)benzyl]-4-pyridin-4-ylpiperidine-3-carboxamide

### Step 1: rac-tert-butyl (3S,4R)-3-({cyclopropyl[3-(2-methoxyethoxy)-5-(3-methoxypropyl)benzyl]amino}carbonyl)-4-hydroxy-4-pyridin-4-ylpiperidine-1-carboxylate

4-Pyridylmagnesium bromide was prepared as follows: to a solution of 4-bromopyridine hydrochloride (1 eq.) in THF (0.146 M) at rt was added isopropylmagnesium chloride (2 eq.). The reaction mixture was stirred at rt 30 min, giving a solution of 4-pyridylmagnesium bromide that was determined to be 0.09 M by reaction with benzaldehyde. Lithium chloride in a round bottom flask was dried under vacuum at 120° C. overnight. It was further flame-dried under vacuum before its use. To lithium chloride (5 eq.) under N₂ was added 4-pyridylmagnesium bromide (0.09M in THF, 3 eq.). The mixture was stirred at RT until all the lithium chloride dissolved (15 min). The mixture was then added dropwise to a solution of keto amide 3.1 (1 eq.) in THF (0.2M) at rt. After 15 min at rt, the reaction was quenched with NH₄Cl and extracted 3× with EtOAc. The combined organic extracts were dried (MgSO₄), filtered and concentrated in vacuo. Purification by automated flash chromatography on silica gel (10-100% EtOAc in hexanes) afforded the title compound.

### Step 2: rac-(3S,4R)—N-cyclopropyl-4-hydroxy-N-[3-(2-methoxyethoxy)-5-(3-methoxypropyl)benzyl]-4-pyridin-4-ylpiperidine-3-carboxamide

To a solution of the title compound from step 1 (1 eq.) in CH₂Cl₂ (0.02 M) was added HCl (4 M in dioxane, 36 eq.). The reaction was stirred at rt for 35 min then 0° C. for 16 h. The reaction was then concentrated in vacuo. Purification by column chromatography on silica gel (5-10% (2M NH₃ in MeOH) in CH₂Cl₂) afforded the title compound as a clear colorless oil. MS: ESI+ve 498.0 (MH+).

### Example 4

### rac-(3S,4R)—N-cyclopropyl-4-(4-fluorophenyl)-4-hydroxy-N-[3-(2-methoxyethoxy)-5-(3-methoxypropyl)benzyl]piperidine-3-carboxamide

### Step 1: rac-tert-butyl (3S,4R)-3-({cyclopropyl[3-(2-methoxyethoxy)-5-(3-methoxypropyl)benzyl]amino}carbonyl)-4-(4-fluorophenyl)-4-hydroxypiperidine-1-carboxylate

Lithium chloride in a round bottom flask was dried under vacuum at 120° C. overnight. It was further flame-dried under vacuum before use. To lithium chloride (3 eq.) at rt was added 4-fluorophenylmagnesium bromide (0.5 M in THF, 3 eq.). The mixture was stirred at rt for 15 min, and added dropwise to a solution of keto amide 3.1 (1 eq.) in THF (0.2M) at rt. After 20 min at rt, the reaction was quenched with NH₄Cl and extracted 3× with EtOAc. The combined organic extracts were dried (MgSO₄), filtered and concentrated in vacuo. Purification by automated flash chromatography on silica gel (20-50% EtOAc in hexanes) afforded the title compound as a clear colorless oil.

### Step 2: rac-(3S,4R)—N-cyclopropyl-4-(4-fluorophenyl)-4-hydroxy-N-[3-(2-methoxyethoxy)-5-(3-methoxypropyl)benzyl]piperidine-3-carboxamide

To a solution of the title compound from step 1 (1 eq.) in CH₂Cl₂ (0.05 M) at rt was added HCl (4 M in dioxane, 38 eq.). The reaction was stirred at rt for 25 min. The reaction was then concentrated in vacuo. Purification by column chromatography on silica gel (5-10% (2M NH₃ in MeOH) in CH₂Cl₂) afforded the title compound as a clear colorless oil. MS: APCI+ve 515.5 (MH+).

### Example 5

### rac-(3S,4R)—N-cyclopropyl-4-(3-fluorophenyl)-4-hydroxy-N-[3-(2-methoxyethoxy)-5-(3-methoxypropyl)benzyl]piperidine-3-carboxamide

### Step 1: rac-tert-butyl (3S,4R)-3-({cyclopropyl[3-(2-methoxyethoxy)-5-(3-methoxypropyl)benzyl]amino}carbonyl)-4-(3-fluorophenyl)-4-hydroxypiperidine-1-carboxylate

Lithium chloride in a round bottom flask was dried under vacuum at 120° C. overnight. It was further flame-dried under vacuum before its use. To lithium chloride (3 eq.) under N₂ was added 3-fluorophenylmagnesium bromide (0.5 M in THF, 3 eq.). The mixture was stirred at rt for 15 min. The mixture was then added dropwise to a solution of keto amide 3.1 (1 eq.) in THF (0.2M) at rt. After 25 min at RT, the reaction was quenched with NH₄Cl and extracted 3× with EtOAc. The combined organic extracts were dried (MgSO₄), filtered and concentrated in vacuo. Purification by automated flash chromatography on silica gel (20-50% EtOAc in hexanes) afforded the title compound as a clear colorless oil.

### Step 2: rac-(3S,4R)—N-cyclopropyl-4-(3-fluorophenyl)-4-hydroxy-N-[3-(2-methoxyethoxy)-5-(3-methoxypropyl)benzyl]piperidine-3-carboxamide

To a solution of the title compound from step 1 (1 eq.) in CH₂Cl₂ (0.05 M) was added HCl (4 M in dioxane, 36 eq.). The reaction was stirred at RT for 25 min. The reaction was then concentrated in vacuo. Purification by column chromatography on silica gel (5-10% (2M NH₃ in MeOH) in CH₂Cl₂) afforded the title compound as a clear colorless oil. MS: ESI+ve 515.1 (MH+).

### Example 6

rac-(3S,4R)—N-cyclopropyl-4-(3,4-difluorophenyl)-4-hydroxy-N-[3-(2-methoxyethoxy)-5-(3-methoxypropyl)benzyl]piperidine-3-carboxamide

### Step 1: rac-tert-butyl (3S,4R)-3-({cyclopropyl[3-(2-methoxyethoxy)-5-(3-methoxypropyl)benzyl]amino}carbonyl)-4-(3,4-difluorophenyl)-4-hydroxypiperidine-1-carboxylate

Lithium chloride in a round bottom flask was dried under vacuum at 120° C. overnight. It was further flame-dried under vacuum before its use. To lithium chloride (3 eq.) under N₂ was added 3,4-difluorophenylmagnesium bromide (0.5 M in THF, 3 eq.). The mixture was stirred at rt for 15 min. The mixture was then added dropwise to a solution of keto amide 3.1 (1 eq.) in THF (0.2M) at rt. After 15 min at rt, the reaction was quenched with NH₄Cl and extracted 3× with EtOAc. The combined organic extracts were dried (MgSO₄), filtered and concentrated in vacuo. Purification by automated flash chromatography on silica gel (20-40% EtOAc in hexanes) afforded the title compound as a white solid.

### Step 2: rac-(3S,4R)—N-cyclopropyl-4-(3,4-difluorophenyl)-4-hydroxy-N-[3-(2-methoxyethoxy)-5-(3-methoxypropyl)benzyl]piperidine-3-carboxamide

To a solution of the title compound from step 1 (1 eq.) in CH₂Cl₂ (0.04 M) was added HCl (4 M in dioxane, 36 eq.). The reaction was stirred at rt for 2 h. The reaction was then concentrated in vacuo. Purification by column chromatography on silica gel (5-10% (2M NH₃ in MeOH) in CH₂Cl₂) afforded the title compound as a clear colorless oil. MS: APCI+ve 533.5 (MH+).

### Example 7

### rac-(3S,4R)—N-cyclopropyl-4-(3,5-difluorophenyl)-4-hydroxy-N-[3-(2-methoxyethoxy)-5-(3-methoxypropyl)benzyl]piperidine-3-carboxamide

### Step 1: rac-tert-butyl (3S,4R)-3-({cyclopropyl[3-(2-methoxyethoxy)-5-(3-methoxypropyl)benzyl]amino}carbonyl)-4-(3,5-difluorophenyl)-4-hydroxypiperidine-1-carboxylate

Lithium chloride in a round bottom flask was dried under vacuum at 120° C. overnight. It was further flame-dried under vacuum before its use. To lithium chloride (3 eq.) under N₂ was added 3,5-difluorophenylmagnesium bromide (0.5 M in THF, 3 eq.). The mixture was stirred at rt for 15 min. The mixture was then added dropwise to a solution of keto amide 3.1 (1 eq.) in THF (0.2M) at rt. After 15 min at rt, the reaction was quenched with NH₄Cl and extracted 3× with EtOAc. The combined organic extracts were dried (MgSO₄), filtered and concentrated in vacuo. Purification by automated flash chromatography on silica gel (20-40% EtOAc in hexanes) afforded the title compound as a clear colorless oil.

### Step 2: rac-(3S,4R)—N-cyclopropyl-4-(3,5-difluorophenyl)-4-hydroxy-N-[3-(2-methoxyethoxy)-5-(3-methoxypropyl)benzyl]piperidine-3-carboxamide

To a solution of the title compound from the previous step (1 eq.) in CH₂Cl₂ (0.03 M) was added HCl (4 M in dioxane, 36 eq.). The reaction was stirred at rt for 45 min. The reaction was then concentrated in vacuo. Purification by automated flash chromatography on silica gel (5-10% (2M NH₃ in MeOH) in CH₂Cl₂) afforded the title compound as a clear colorless oil. MS: ESI+ve 533.1 (MH+).

### Example 8

### rac-(3S,4R)-4-(3-chlorophenyl)-N-cyclopropyl-4-hydroxy-N-[3-(2-methoxyethoxy)-5-(3-methoxypropyl)benzyl]piperidine-3-carboxamide

### Step 1: rac-tert-butyl (3S,4R)-4-(3-chlorophenyl)-3-({cyclopropyl[3-(2-methoxyethoxy)-5-(3-methoxypropyl)benzyl]amino}carbonyl)-4-hydroxypiperidine-1-carboxylate

Lithium chloride in a round bottom flask was dried under vacuum at 120° C. overnight. It was further flame-dried under vacuum before its use. To lithium chloride (3 eq.) under N₂ was added 3-chlorophenylmagnesium bromide (0.5 M in THF, 3 eq.). After stirring for 15 min at rt, the mixture was then added dropwise to a solution of keto amide 3.1 (1 eq.) in THF (0.2M) at rt. After 20 min at rt, the reaction was quenched with NH₄Cl and extracted 3× with EtOAc. The combined organic extracts were dried (MgSO₄), filtered and concentrated in vacuo. Purification by automated flash chromatography on silica gel (20-40% EtOAc in hexanes) afforded the title compound as a clear colorless oil.

### Step 2: rac-(3S,4R)-4-(3-chlorophenyl)-N-cyclopropyl-4-hydroxy-N-[3-(2-methoxyethoxy)-5-(3-methoxypropyl)benzyl]piperidine-3-carboxamide

To a solution of the title compound from the previous step (1 eq.) in CH₂Cl₂ (0.05 M) was added HCl (4 M in dioxane, 45 eq.). The reaction was stirred at rt for 25 min. The reaction was then concentrated in vacuo. Purification by column chromatography on silica gel (5-10% (2M NH₃ in MeOH) in CH₂Cl₂) afforded the title compound as a clear colorless oil. MS: ESI+ve 531.0 (MH+).

### Example 9

### rac-(3S,4R)-4-(4-chlorophenyl)-N-cyclopropyl-4-hydroxy-N-[3-(2-methoxyethoxy)-5-(3-methoxypropyl)benzyl]piperidine-3-carboxamide

### Step 1: rac-tert-butyl (3S,4R)-4-(4-chlorophenyl)-3-({cyclopropyl[3-(2-methoxyethoxy)-5-(3-methoxypropyl)benzyl]amino}carbonyl)-4-hydroxypiperidine-1-carboxylate

Lithium chloride in a round bottom flask was dried under vacuum at 120° C. overnight. It was further flame-dried under vacuum before its use. To lithium chloride (3 eq.) under N₂ was added 4-chlorophenylmagnesium bromide (1.0 M in THF, 6 eq.). The mixture was stirred at rt until all of the lithium chloride dissolved. The mixture was then added dropwise to a solution of keto amide 3.1 (1 eq.) in THF (0.2M) at rt. After 14 min at rt, the reaction was quenched with NH₄Cl and extracted 3× with EtOAc. The combined organic extracts were dried (MgSO₄), filtered and concentrated in vacuo. Purification by automated flash chromatography on silica gel (20-40% EtOAc in hexanes) afforded the title compound as a clear colorless oil.

### Step 2: rac-(3S,4R)-4-(4-chlorophenyl)-N-cyclopropyl-4-hydroxy-N-[3-(2-methoxyethoxy)-5-(3-methoxypropyl)benzyl]piperidine-3-carboxamide

To a solution of the title compound from the previous step (1 eq.) in CH₂Cl₂ (0.03 M) was added HCl (4 M in dioxane, 36 eq.). The reaction was stirred at rt for 25 min. The reaction was then concentrated in vacuo. Purification by column chromatography on silica gel (5-10% (2M NH₃ in MeOH) in CH₂Cl₂) afforded the title compound as a clear colorless oil. MS: APCI+ve 531.5 (MH+).

### Example 10

### rac-(3S,4R)-4-(4-chloro-3-fluorophenyl)-N-cyclopropyl-4-hydroxy-N-[3-(2-methoxyethoxy)-5-(3-methoxypropyl)benzyl]piperidine-3-carboxamide

### Step 1: rac-tert-butyl (3S,4R)-4-(4-chloro-3-fluorophenyl)-3-({cyclopropyl[3-(2-methoxyethoxy)-5-(3-methoxypropyl)benzyl]amino}carbonyl)-4-hydroxypiperidine-1-carboxylate

Lithium chloride in a round bottom flask was dried under vacuum at 120° C. overnight. It was further flame-dried under vacuum before its use. To lithium chloride (3 eq.) under N₂ was added 4-chloro-3-fluoro-phenylmagnesium bromide (0.5 M in THF, 3 eq.). The mixture was stirred at RT until all of the lithium chloride dissolved. The mixture was then added dropwise to a solution of keto amide 3.1 (1 eq.) in THF (0.2M) at rt. After 15 min at rt, the reaction was quenched with NH₄Cl and extracted 2× with Et₂O. The combined organic extracts were dried (MgSO₄), filtered and concentrated in vacuo. Purification by automated flash chromatography on silica gel (20-50% EtOAc in hexanes) afforded the title compound as a clear colorless oil.

### Step 2: rac-(3S,4R)-4-(4-chloro-3-fluorophenyl)-N-cyclopropyl-4-hydroxy-N-[3-(2-methoxyethoxy)-5-(3-methoxypropyl)benzyl]piperidine-3-carboxamide

To a solution of the title compound from the previous step (1 eq.) in CH₂Cl₂ (0.06 M) was added HCl (4 M in dioxane, 36 eq.). The reaction was stirred at rt for 25 min. The reaction was then concentrated in vacuo. Purification by column chromatography on silica gel (5-10% (2M NH₃ in MeOH) in CH₂Cl₂) afforded the title compound as a clear colorless oil. MS: ESI+ve 549.0 (MH+).

### Example 11

### rac-(3S,4R)—N-cyclopropyl-4-(3,4-dichlorophenyl)-4-hydroxy-N-[3-(2-methoxyethoxy)-5-(3-methoxypropyl)benzyl]piperidine-3-carboxamide

### Step 1: rac-tert-butyl (3S,4R)-3-({cyclopropyl[3-(2-methoxyethoxy)-5-(3-methoxypropyl)benzyl]amino}carbonyl)-4-(3,4-dichlorophenyl)-4-hydroxypiperidine-1-carboxylate

Lithium chloride in a round bottom flask was dried under vacuum at 120° C. overnight. It was further flame-dried under vacuum before its use. To lithium chloride (3 eq.) under N₂ at rt was added 3,4-dichlorophenylmagnesium bromide (0.5 M in THF, 3 eq.). After stirring for 45 min rt, the mixture was added dropwise to a solution of keto amide 3.1 (1 eq.) in THF (0.2M) at rt. After 15 min at rt, the reaction was quenched with NH₄Cl and extracted with Et₂O and EtOAc. The combined organic extracts were dried (MgSO₄), filtered and concentrated in vacuo. Purification by automated flash chromatography on silica gel (20-40% EtOAc in hexanes) afforded the title compound.

### Step 2: rac-(3S,4R)—N-cyclopropyl-4-(3,4-dichlorophenyl)-4-hydroxy-N-[3-(2-methoxyethoxy)-5-(3-methoxypropyl)benzyl]piperidine-3-carboxamide

To a solution of the title compound from step 1 (1 eq.) in CH₂Cl₂ (0.05 M) at rt was added HCl (4 M in dioxane, 36 eq.). The reaction was stirred at rt for 30 min and concentrated in vacuo. Purification by column chromatography on silica gel (5-10% (2M NH₃ in MeOH) in CH₂Cl₂) afforded the title compound as a clear colorless oil. MS: ESI+ve 565.1 (MH+).

### Example 12

### rac-(3S,4R)—N-cyclopropyl-4-hydroxy-N-[3-(2-methoxyethoxy)-5-(3-methoxypropyl)benzyl]-4-(2-methoxyphenyl)piperidine-3-carboxamide

### Step 1: rac-tert-butyl (3S,4R)-3-({cyclopropyl[3-(2-methoxyethoxy)-5-(3-methoxypropyl)benzyl]amino}carbonyl)-4-hydroxy-4-(2-methoxyphenyl)piperidine-1-carboxylate

To a solution of keto amide 3.1 (1.0 eq.) in THF (0.1 M) at rt under N₂ was added commercially available 2-methoxyphenylmagnesium chloride (0.5 M in THF, 2.0 eq.). The reaction was stirred at rt for 15 min. The reaction was then quenched with NH₄Cl and extracted 3× with EtOAc. The combined organic extracts were dried (MgSO₄), filtered and concentrated in vacuo. Purification by automated flash chromatography on silica gel (20-70% EtOAc in hexanes) afforded the title compound.

### Step 2: rac-(3S,4R)—N-cyclopropyl-4-hydroxy-N-[3-(2-methoxyethoxy)-5-(3-methoxypropyl)benzyl]-4-(2-methoxyphenyl)piperidine-3-carboxamide

To a solution of the title compound from step 1 (1 eq.) in CH₂Cl₂ (0.05 M) was added HCl (4 M in dioxane, 36 eq.). The reaction was stirred at rt for 45 min. The reaction was then concentrated in vacuo. Purification by automated flash chromatography on silica gel (5-10% (2M NH₃ in MeOH) in CH₂Cl₂) afforded the title compound as a clear colorless oil. MS: ESI+ve 527.2 (MH+).

### Example 13

### rac-(3S,4R)-4-[4-chloro-3-(trifluoromethyl)phenyl]-N-cyclopropyl-4-hydroxy-N-[3-(2-methoxyethoxy)-5-(3-methoxypropyl)benzyl]piperidine-3-carboxamide

### Step 1: tert-butyl (3S,4R)-4-[4-chloro-3-(trifluoromethyl)phenyl]-3-({cyclopropyl[3-(2-methoxyethoxy)-5-(3-methoxypropyl)benzyl]amino}carbonyl)-4-hydroxypiperidine-1-carboxylate

n-BuLi (2.50 M in hexanes, 3.03 eq.) was added to a stirred solution of 4-bromo-1-chloro-2-(trifluoromethyl)benzene (3.11 eq.) in THF (0.4 M) at −78° C. The mixture was stirred at −78° C. for 15 min then MgBr₂.Et₂O (0.4 M in THF, 3.18 eq.) was added dropwise. The mixture was stirred −78° C. for 30 min. The resulting solution was cannulated into a solution of keto amide 3.1 (1 eq.) in THF (0.15 M) at −78° C. The final reaction mixture was stirred at −78° C. for 1 hr then allow to warm slowly to rt with stirring over 12 h. The mixture was quenched with saturated NH₄Cl and diluted with Et₂O. The organic extract was washed with brine, dried (MgSO₄), filtered and concentrated in vacuo. The residue was purified by column chromatography on silica gel (10-100% EtOAc in hexanes) to give the title compound as an oil.

### Step 2: rac-(3S,4R)-4-[4-chloro-3-(trifluoromethyl)phenyl]-N-cyclopropyl-4-hydroxy-N-[3-(2-methoxyethoxy)-5-(3-methoxypropyl)benzyl]piperidine-3-carboxamide

To a solution of the title compound from step 1 (1 eq.) in CH₂Cl₂ (0.03 M) at rt was added HCl (4 M in dioxane, 30 eq.). The reaction was stirred at rt for 1 h. The reaction was then concentrated in vacuo. Purification by automated flash chromatography on silica gel (5-10% (2M NH₃ in MeOH) in CH₂Cl₂) afforded the title compound as a clear colorless oil. MS: ESI+ve 599.0 (MH+).

### Example 14

### rac-(3S,4R)—N-cyclopropyl-4-[2-fluoro-4-(trifluoromethyl)phenyl]-4-hydroxy-N-[3-(2-methoxyethoxy)-5-(3-methoxypropyl)benzyl]piperidine-3-carboxamide

### Step 1: rac-tert-butyl (3S,4R)-3-({cyclopropyl[3-(2-methoxyethoxy)-5-(3-methoxypropyl)benzyl]amino}carbonyl)-4-[2-fluoro-4-(trifluoromethyl)phenyl]-4-hydroxypiperidine-1-carboxylate

n-BuLi (2.50 M in THF, 3.11 eq.) was added to a stirred solution of 1-bromo-2-fluoro-4-(trifluoromethyl)benzene (3.24 eq.) in THF (1.6 M) at −78° C. The mixture was stirred at −78° C. for 15 min then MgBr₂.Et₂O (0.4 M in THF, 3.32 eq.) was added dropwise. The mixture was stirred −78° C. for 30 min. The resulting solution was cannulated into a solution of keto amide 3.1 (1 eq.) in THF (0.15 M) at −78° C. The reaction mixture was stirred at −78° C. for 1 hr then allow to warm slowly to rt with stirring over 12 h. The mixture was quenched with saturated NH₄Cl, diluted with Et₂O. The organic extract was washed with brine, dried (MgSO₄), filtered and concentrated in vacuo. The residue was purified by column chromatography on silica gel (10-75% EtOAc in hexanes) to give the title compound as an oil.

### Step 2: rac-(3S,4R)—N-cyclopropyl-4-[2-fluoro-4-(trifluoromethyl)phenyl]-4-hydroxy-N-[3-(2-methoxyethoxy)-5-(3-methoxypropyl)benzyl]piperidine-3-carboxamide

To a solution of the title compound from step 1 (1 eq.) in CH₂Cl₂ (0.03 M) at rt was added HCl (4 M in dioxane, 29 eq.). The reaction was stirred at rt for 1 h. The reaction was then concentrated in vacuo. Purification by automated flash chromatography on silica gel (4% (2M NH₃ in MeOH) in CH₂Cl₂) afforded the title compound as a colorless oil. MS: ESI+ve 583.0 (MH+).

### Example 15

### rac-(3S,4R)—N-cyclopropyl-4-hydroxy-N-[3-(2-methoxyethoxy)-5-(3-methoxypropyl)benzyl]-4-(3-methoxyphenyl)piperidine-3-carboxamide

### Step 1: rac-tert-butyl (3S,4R)-3-({cyclopropyl[3-(2-methoxyethoxy)-5-(3-methoxypropyl)benzyl]amino}carbonyl)-4-hydroxy-4-(3-methoxyphenyl)piperidine-1-carboxylate

To a solution of ketoamide 3.1 (1 eq.) in THF (0.2 M) at 0° C. was added flame-dried lithium chloride (5 eq.). A solution of 3-methoxyphenylmagnesium bromide (1 M in THF, 3 eq.) was then added and the mixture stirred at 0° C. for 30 min then rt for 15 min. The reaction was then quenched with H₂O and extracted with EtOAc. The organic extract was washed with brine, dried (MgSO₄), filtered and evaporated in vacuo. Purification by flash chromatography on silica gel (10% acetone in toluene) afforded the title compound.

### Step 2: rac-(3S,4R)—N-cyclopropyl-4-hydroxy-N-[3-(2-methoxyethoxy)-5-(3-methoxypropyl)benzyl]-4-(3-methoxyphenyl)piperidine-3-carboxamide

To a solution of rac-tert-butyl (3S,4R)-3-({cyclopropyl[3-(2-methoxyethoxy)-5-(3-methoxypropyl)benzyl]amino}carbonyl)-4-hydroxy-4-(3-methoxyphenyl)piperidine-1-carboxylate (1 eq.) from the previous step in CH₂Cl₂ (0.1 M) was added HCl (4 M in dioxane, 10 eq.). The reaction was stirred at rt for 4 h. The reaction was then concentrated in vacuo. Purification by automated flash chromatography on silica gel (5% (2M NH₃ in MeOH) in CH₂Cl₂) afforded the title compound as a colorless oil. MS: ESI+ve 527.2 (MH+).

### Example 16

### rac-(3S,4R)-4-(3-aminophenyl)-N-cyclopropyl-4-hydroxy-N-[3-(2-methoxyethoxy)-5-(3-methoxypropyl)benzyl]piperidine-3-carboxamide

### Step 1: rac-tert-butyl (3S,4R)-4-{3-[bis(trimethylsilyl)amino]phenyl}-3-({cyclopropyl[3-(2-methoxyethoxy)-5-(3-methoxypropyl)benzyl]amino}carbonyl)-4-hydroxypiperidine-1-carboxylate

To a solution of ketoamide 3.1 (1 eq.) in THF (0.2 M) at 0 C was added flame-dried lithium chloride (5 eq.). A solution of 3-[bis(trimethylsilyl)amino]phenylmagnesium bromide (3 eq.) was then added and the mixture stirred at 0° C. for 30 min then rt for 15 min. The reaction was then quenched with H₂O and extracted with EtOAc. The organic extract was washed with brine, dried (MgSO₄), filtered and evaporated in vacuo. Purification by flash chromatography on silica gel (10% acetone in toluene) afforded the title compound.

### Step 2: rac-(3S,4R)-4-(3-aminophenyl)-N-cyclopropyl-4-hydroxy-N-[3-(2-methoxyethoxy)-5-(3-methoxypropyl)benzyl]piperidine-3-carboxamide

To a solution of rac-tert-butyl (3S,4R)-4-{3-[bis(trimethylsilyl)amino]phenyl}-3-({cyclopropyl[3-(2-methoxyethoxy)-5-(3-methoxypropyl)benzyl]amino}carbonyl)-4-hydroxypiperidine-1-carboxylate (1 eq.) from the previous step in CH₂Cl₂ (0.1 M) at rt was added HCl (4 M in dioxane, 10 eq.). The reaction was stirred at rt for 5 h. The reaction was then concentrated in vacuo. Purification by automated flash chromatography on silica gel (10% (2M NH₃ in MeOH) in CH₂Cl₂) afforded the title compound as a colorless oil. MS: ESI+ve 512.1 (MH+).

### Example 17

### rac-(3S,4R)—N-cyclopropyl-4-hydroxy-N-[3-(2-methoxyethoxy)-5-(3-methoxypropyl)benzyl]-4-(1,3-thiazol-2-yl)piperidine-3-carboxamide

### Step 1: rac-tert-butyl (3S,4R)-3-({cyclopropyl[3-(2-methoxyethoxy)-5-(3-methoxypropyl)benzyl]amino}carbonyl)-4-hydroxy-4-(1,3-thiazol-2-yl)piperidine-1-carboxylate

n-BuLi (2.5 M in hexanes, 1.45 eq.) was added to a stirred solution of thiazole (1.46 eq.) in THF (0.14 M) at −78° C. The mixture was stirred at −78° C. for 15 min, then magnesium bromide diethyl etherate (0.5 M in THF, 1.50 eq.) was added dropwise. The mixture was stirred at −78° C. for 1 h. To this mixture was then added via canula a solution of ketoamide 3.1 (1 eq.) in THF (0.1 M) at −78° C. The final reaction mixture was stirred at −78° C. for 1 hr then at −10° C. for 1 h. The mixture was quenched with saturated NH₄Cl, and extracted with EtOAc. The organic extract was washed with brine, dried (MgSO₄), filtered and concentrated in vacuo. The residue was purified by column chromatography on silica gel (25% acetone in toluene) to afford the title compound.

### Step 2: rac-(3S,4R)—N-cyclopropyl-4-hydroxy-N-[3-(2-methoxyethoxy)-5-(3-methoxypropyl)benzyl]-4-(1,3-thiazol-2-yl)piperidine-3-carboxamide

To a solution of the title compound from step 1 (1 eq.) in CH₂Cl₂ (0.02 M) at rt was added HCl (4 M in dioxane, 48 eq.). The reaction was stirred at rt for 1 h. The reaction was then concentrated in vacuo. Purification by automated flash chromatography on silica gel (4-8% (2M NH₃ in MeOH) in CH₂Cl₂) afforded the title compound as a colorless oil. MS: ESI+ve 504.1 (MH+).

### Example 18

### rac-(3S,4R)—N-cyclopropyl-4-hydroxy-N-[3-(2-methoxyethoxy)-5-(3-methoxypropyl)benzyl]-4-(1-methyl-1H-imidazol-2-yl)piperidine-3-carboxamide

### Step 1: rac-tert-butyl (3S,4R)-3-({cyclopropyl[3-(2-methoxyethoxy)-5-(3-methoxypropyl)benzyl]amino}carbonyl)-4-hydroxy-4-(1-methyl-1H-imidazol-2-yl)piperidine-1-carboxylate

n-BuLi (2.5 M in hexanes, 2.98 eq.) was added to a stirred solution of 1-methylimidazole (3.0 eq.) in THF (0.3 M) at −78° C. The mixture was stirred at −78° C. for 15 min then magnesium bromide diethyl etherate (0.5 M in THF, 3.11 eq.) was added dropwise. The mixture was stirred at −78° C. for 30 min. The resulting solution was cannulated into a solution of ketoamide 3.1 (1 eq.) in THF (0.1 M) at −78° C. The reaction mixture was stirred at −78° C. for 1 hr then allow to warm slowly to rt with stirring over 12 h, quenched with saturated NH₄Cl, and extracted with EtOAc. The organic extract was washed with brine, dried (MgSO₄), filtered and concentrated in vacuo. The residue was purified by column chromatography on silica gel (10-100% EtOAc in hexanes) to give the title compound as a colorless oil.

### Step 2: rac-(3S,4R)—N-cyclopropyl-4-hydroxy-N-[3-(2-methoxyethoxy)-5-(3-methoxypropyl)benzyl]-4-(1-methyl-1H-imidazol-2-yl)piperidine-3-carboxamide

To a solution of rac-1-methyl-1H-imidazol-2-yl alcohol (1 eq.) from the previous step in CH₂Cl₂ (0.03 M) at rt was added HCl (4 M in dioxane, 30 eq.). The reaction was stirred at rt for 1 h. The reaction was then concentrated in vacuo. Purification by automated flash chromatography on silica gel (4% (2M NH₃ in MeOH) in CH₂Cl₂) afforded the title compound as a colorless oil. MS: ESI+ve 501.3 (MH+).

### Example 19

### rac-(3S,4R)—N-cyclopropyl-4-hydroxy-N-[3-(2-methoxyethoxy)-5-(3-methoxypropyl)benzyl]-4-(1H-1,2,3-triazol-4-yl)piperidine-3-carboxamide

### Step 1: rac-tert-butyl (3S,4R)-3-({cyclopropyl[3-(2-methoxyethoxy)-5-(3-methoxypropyl)benzyl]amino}carbonyl)-4-ethynyl-4-hydroxypiperidine-1-carboxylate

To a solution of ketoamide 3.1 (1 eq.) in THF (0.2 M) at 0° C. was added flame-dried lithium chloride (5 eq.). A solution of ethynylmagnesium bromide (0.5 M in THF, 3 eq.) was then added and the final mixture stirred at 0° C. for 30 min then rt. The reaction was then quenched with H₂O and extracted with EtOAc. The organic extract was washed with brine, dried (MgSO₄), filtered and evaporated in vacuo. Purification by flash chromatography on silica gel (10% acetone in toluene) afforded the title compound.

### Step 2: rac-tert-butyl (3S,4R)-3-({cyclopropyl[3-(2-methoxyethoxy)-5-(3-methoxypropyl)benzyl]amino}carbonyl)-4-hydroxy-4-(1H-1,2,3-triazol-4-yl)piperidine-1-carboxylate

A mixture of the title compound from step 1 (1 eq.), trimethylsilyl azide (1.5 eq.) and CuI (0.05 eq.) in a mixture of DMF and MeOH (9:1 respectively, 0.18 M solution) was heated to 100° C. 16 h, cooled to rt, diluted with EtOAc, washed with water and brine, dried over MgSO₄, filtered and concentrated in vacuo. The residue was purified by flash chromatography (SiO₂; 20% acetone in toluene) to afford the title compound.

### Step 3: rac-(3S,4R)—N-cyclopropyl-4-hydroxy-N-[3-(2-methoxyethoxy)-5-(3-methoxypropyl)benzyl]-4-(1H-1,2,3-triazol-4-yl)piperidine-3-carboxamide

To a solution of rac-tert-butyl (3S,4R)-3-({cyclopropyl[3-(2-methoxyethoxy)-5-(3-methoxypropyl)benzyl]amino}carbonyl)-4-hydroxy-4-(1H-1,2,3-triazol-4-yl)piperidine-1-carboxylate (1 eq.) from step 2 in CH₂Cl₂ (0.06 M) was added HCl (4 M in dioxane, 10 eq.). The reaction was stirred at rt for 4 h. The reaction was then concentrated in vacuo. Purification by automated flash chromatography on silica gel (10% (2M NH₃ in MeOH) in CH₂Cl₂) afforded the title compound as a colorless oil. MS: ESI+ve 488.2 (MH+).

### Example 20

### rac-(3S,4R)—N-cyclopropyl-4-hydroxy-N-[3-(2-methoxyethoxy)-5-(3-methoxypropyl)benzyl]-4-(2-thienyl)piperidine-3-carboxamide

### Step 1: rac-tert-butyl (3S,4R)-3-({cyclopropyl[3-(2-methoxyethoxy)-5-(3-methoxypropyl)benzyl]amino}carbonyl)-4-hydroxy-4-(2-thienyl)piperidine-1-carboxylate

Lithium chloride in a round bottom flask was dried under vacuum at 120° C. overnight. It was further flame-dried under vacuum before its use. To lithium chloride (5 eq.) under N₂ was added 2-thienylmagnesium bromide (1 M in THF, 3 eq.). The mixture was stiffed at rt for a few minutes to dissolve the lithium chloride then cooled to 0° C. The mixture was then added dropwise to a solution of keto amide 3.1 (1 eq.) in THF (0.2M) at 0° C. After 30 min at 0° C., the mixture was allowed to warm to rt then quenched H₂O and extracted with EtOAc. The organic extract was washed with brine, dried (MgSO₄), filtered and concentrated in vacuo. Purification by flash chromatography on silica gel (10% acetone in toluene) afforded the title compound.

### Step 2: rac-(3S,4R)—N-cyclopropyl-4-hydroxy-N-[3-(2-methoxyethoxy)-5-(3-methoxypropyl)benzyl]-4-(2-thienyl)piperidine-3-carboxamide

To a solution of rac-tert-butyl (3S,4R)-3-({cyclopropyl[3-(2-methoxyethoxy)-5-(3-methoxypropyl)benzyl]amino}carbonyl)-4-hydroxy-4-(2-thienyl)piperidine-1-carboxylate (1 eq.) from step 1 in CH₂Cl₂ (0.1 M) at rt was added HCl (4 M in dioxane, 10 eq.). The reaction was stirred at rt for 4 h. The reaction was then concentrated in vacuo. Purification by automated flash chromatography on silica gel (10% (2M NH₃ in MeOH) in CH₂Cl₂) afforded the title compound as a colorless oil. MS: ESI+ve 503.3 (MH+).

### Example 21

### rac-(3S,4R)-4-(1,3-benzoxazol-2-yl)-N-cyclopropyl-4-hydroxy-N-[3-(2-methoxyethoxy)-5-(3-methoxypropyl)benzyl]piperidine-3-carboxamide

### Step 1: rac-tert-butyl (3S,4R)-4-(1,3-benzoxazol-2-yl)-3-({cyclopropyl[3-(2-methoxyethoxy)-5-(3-methoxypropyl)benzyl]amino}carbonyl)-4-hydroxypiperidine-1-carboxylate

To a solution of benzoxazole (3 eq.) in THF (0.6M) at −78° C. was added n-BuLi (2.5M in hexanes, 3 eq.). After 30 min, a solution of magnesium bromide diethyl etherate (0.5 M in THF, 3 eq.) was added and the mixture stirred at −78° C. for 30 min. This mixture was then added to a solution of ketoamide 3.1 (1 eq.) in THF (0.2 M) at −78° C. The reaction mixture was then allowed to warm slowly to rt over 16 h. The reaction was then quenched with H₂O and extracted with EtOAc. The organic extract was washed with brine, dried (MgSO₄), filtered and concentrated in vacuo. Purification by flash chromatography on silica gel (10% acetone in toluene) afforded the title compound.

### Step 2: rac-(3S,4R)-4-(1,3-benzoxazol-2-yl)-N-cyclopropyl-4-hydroxy-N-[3-(2-methoxyethoxy)-5-(3-methoxypropyl)benzyl]piperidine-3-carboxamide

To a solution of rac-tert-butyl (3S,4R)-4-(1,3-benzoxazol-2-yl)-3-({cyclopropyl[3-(2-methoxyethoxy)-5-(3-methoxypropyl)benzyl]amino}carbonyl)-4-hydroxypiperidine-1-carboxylate (1 eq.) from step 1 in CH₂Cl₂ (0.03 M) at rt was added HCl (4 M in dioxane, 10 eq.). The reaction was stirred at rt for 4 h, and concentrated in vacuo. Purification by automated flash chromatography on silica gel (10% (2M NH₃ in MeOH) in CH₂Cl₂) afforded the title compound as a colorless oil. MS: ESI+ve 539.7 (MH+).

### Example 22

### rac-(3S,4R)-4-[2-(benzyloxy)pyridin-4-yl]-N-cyclopropyl-4-hydroxy-N-[3-(2-methoxyethoxy)-5-(3-methoxypropyl)benzyl]piperidine-3-carboxamide

### Step 1: 2-(benzyloxy)-4-bromopyridine

A flame-dried round bottom was charged with 4-bromopyridin-2-ol (1 eq.), benzene (0.14 M), Ag₂CO₃ (0.6 eq.) and benzyl bromide (1.2 eq.) under N₂. The reaction was heated to 55° C. overnight in the dark. The reaction was then cooled and filtered, washing with dichloromethane. The filtrate was concentrated in vacuo and the residue was purified by flash chromatography (SiO₂; 2-4% Et₂O in hexanes) to give the desired product as a clear oil.

### Step 2: rac-tert-butyl (3S,4R)-4-[2-(benzyloxy)pyridin-4-yl]-3-({cyclopropyl[3-(2-methoxyethoxy)-5-(3-methoxypropyl)benzyl]amino}carbonyl)-4-hydroxypiperidine-1-carboxylate

To a solution of 2-(benzyloxy)-4-bromopyridine from the previous step (1 eq.) in THF (0.1M) at −78° C. was added n-BuLi (2.5 M in hexanes, 2.1 eq.). The mixture was stirred for 30 min at −78° C. MgBr₂ (solid, 2.5 eq.) was added, and the resulting mixture stirred at −78° C. for 20 min, and 30 min at 0° C. A solution of ketoamide 3.1 in THF (0.04 M) was added and the resulting mixture stirred at 0° C. for 1 h and rt for 0.5 h. The reaction was quenched with saturated aqueous NH₄Cl solution and extracted 2×Et₂O. The combined organic extracts were washed with brine, dried (MgSO₄), filtered and concentrated in vacuo. The residue was purified by automated flash chromatography (SiO₂; 0-15% acetone in toluene) to afford the title compound as a clear colorless oil.

### Step 3: rac-(3S,4R)-4-[2-(benzyloxy)pyridin-4-yl]-N-cyclopropyl-4-hydroxy-N-[3-(2-methoxyethoxy)-5-(3-methoxypropyl)benzyl]piperidine-3-carboxamide

To a solution of rac-tert-butyl (3S,4R)-4-[2-(benzyloxy)pyridin-4-yl]-3-({cyclopropyl[3-(2-methoxyethoxy)-5-(3-methoxypropyl)benzyl]amino}carbonyl)-4-hydroxypiperidine-1-carboxylate (1 eq.) from step 2 in CH₂Cl₂ (0.02 M) was added HCl (4 M in dioxane, 30 eq.). The reaction was stirred at rt for 2 h. The reaction was then concentrated in vacuo. Purification by reverse-phase HPLC (C₁₈; 5-95% MeCN in water+0.1% TFA) afforded a salt which was extracted with EtOAc from NaHCO₃ (aq. sat.), dried over MgSO₄, filtered and concentrated in vacuo, to afford the title compound as a clear colorless oil. MS: ESI+ve 604.3 (MH+).

### Example 23

### rac-(3S,4R)—N-cyclopropyl-4-hydroxy-N-[3-(2-methoxyethoxy)-5-(3-methoxypropyl)benzyl]-4-(1-methyl-2-oxo-1,2-dihydropyridin-4-yl)piperidine-3-carboxamide

### Step 1: rac-tert-butyl (3S,4R)-3-({cyclopropyl[3-(2-methoxyethoxy)-5-(3-methoxypropyl)benzyl]amino}carbonyl)-4-hydroxy-4-(2-hydroxypyridin-4-yl)piperidine-1-carboxylate

A solution of rac-tert-butyl (3S,4R)-4-[2-(benzyloxy)pyridin-4-yl]-3-({cyclopropyl[3-(2-methoxyethoxy)-5-(3-methoxypropyl)benzyl]amino}carbonyl)-4-hydroxypiperidine-1-carboxylate from Example 22; step 2 (1 eq.) and acetic acid (1.64 eq.) in EtOAc (0.02M) was hydrogenated over 10% Pd/C (0.4 eq.) at rt for 4.5 h. The catalyst was filtered over celite, washing with CH₂Cl₂, and the filtrate concentrated in vacuo to afford the title compound.

### Step 2: rac-tert-butyl (3S,4R)-3-({cyclopropyl[3-(2-methoxyethoxy)-5-(3-methoxypropyl)benzyl]amino}carbonyl)-4-hydroxy-4-(1-methyl-2-oxo-1,2-dihydropyridin-4-yl)piperidine-1-carboxylate

To a solution of rac-tert-butyl (3S,4R)-3-({cyclopropyl[3-(2-methoxyethoxy)-5-(3-methoxypropyl)benzyl]amino}carbonyl)-4-hydroxy-4-(2-hydroxypyridin-4-yl)piperidine-1-carboxylate from the previous step (1 eq.) in MeOH (0.04M) at 0° C. was added 2M NaOH (3 eq.) followed by dimethylsulfate (4.36 eq.). The reaction was allowed to slowly warm to rt overnight. The solvent was evaporated in vacuo and the resulting residue was purified by column chromatography (3% (2M NH₃ in MeOH) in CH₂Cl₂) to afford the title compound.

### Step 3: rac-(3S,4R)—N-cyclopropyl-4-hydroxy-N-[3-(2-methoxyethoxy)-5-(3-methoxypropyl)benzyl]-4-(1-methyl-2-oxo-1,2-dihydropyridin-4-yl)piperidine-3-carboxamide

To a solution of rac-tert-butyl (3S,4R)-3-({cyclopropyl[3-(2-methoxyethoxy)-5-(3-methoxypropyl)benzyl]amino}carbonyl)-4-hydroxy-4-(1-methyl-2-oxo-1,2-dihydropyridin-4-yl)piperidine-1-carboxylate (1 eq.) from the previous step in CH₂Cl₂ (0.02 M) was added HCl (4 M in dioxane, 30 eq.). The reaction was stirred at rt for 2 h. The reaction was then concentrated in vacuo. Purification by reverse phase HPLC (C₁₈; 5-95% MeCN in water+0.1% TFA) afforded a salt which was extracted with EtOAc from NaHCO₃ (aq. sat.), dried over MgSO₄, filtered and concentrated in vacuo, to afford the title compound as a clear colorless oil. MS: ESI+ve 527.9 (MH+).

### Example 24

### rac-(3S,4R)—N-cyclopropyl-4-hydroxy-N-[3-(2-methoxyethoxy)-5-(3-methoxypropyl)benzyl]-4-(1-methyl-6-oxo-1,6-dihydropyridin-3-yl)piperidine-3-carboxamide

### Step 1: 2-(benzyloxy)-5-bromopyridine

A flame-dried round bottom was charged with 5-bromopyridin-2-ol (1 eq.), benzene (0.19 M), Ag₂CO₃ (0.6 eq.) and benzyl bromide (1.2 eq.) under N₂. The reaction was heated to 50° C. overnight in the dark. The reaction was then cooled and filtered, washing with dichloromethane. The filtrate was concentrated in vacuo and the residue was purified by flash chromatography (2-4% Et₂O in hexanes) to afford the title compound as a white solid.

### Step 2: rac-tert-butyl (3S,4R)-4-[6-(benzyloxy)pyridin-3-yl]-3-({cyclopropyl[3-(2-methoxyethoxy)-5-(3-methoxypropyl)benzyl]amino}carbonyl)-4-hydroxypiperidine-1-carboxylate

To a solution of 2-(benzyloxy)-5-bromopyridine from step 1 (2.26 eq.) in THF (0.1M) at −78° C. was added n-butyllithium (2.5 M in hexanes, 2.3 eq.). The mixture was stirred for 30 min at −78° C. and MgBr₂ (0.5 M in Et₂O, 2.5 eq.) was added. After 30 min at −78° C., the mixture was added dropwise via syringe to a −78° C. solution of ketoamide 3.1 in THF (0.05 M, 1 eq.) and warmed to 0° C. over 14 h. The reaction was quenched with saturated aqueous NaHCO₃ and extracted with 2×EtOAc. The combined organic extracts were washed with brine, dried (MgSO₄), filtered and concentrated in vacuo. The crude was then purified by automated flash chromatography (10-80% EtOAc in hexanes) to afford the title compound.

### Step 3: rac-tert-butyl (3S,4R)-3-({cyclopropyl[3-(2-methoxyethoxy)-5-(3-methoxypropyl)benzyl]amino}carbonyl)-4-hydroxy-4-(6-hydroxypyridin-3-yl)piperidine-1-carboxylate

A solution of rac-tert-butyl (3S,4R)-4-[6-(benzyloxy)pyridin-3-yl]-3-({cyclopropyl[3-(2-methoxyethoxy)-5-(3-methoxypropyl)benzyl]amino}carbonyl)-4-hydroxypiperidine-1-carboxylate prepared from step 2 (1 eq.) and acetic acid (1.2 eq.) in EtOAc (0.02M) was hydrogenated over 10% Pd/C (0.4 eq.) under an atmosphere of hydrogen at rt for 4.5 h. The reaction mixture was filtered through celite, washing with CH₂Cl₂ and the filtrate concentrated in vacuo, to afford the title compound.

### Step 4: rac-tert-butyl (3S,4R)-3-({cyclopropyl[3-(2-methoxyethoxy)-5-(3-methoxypropyl)benzyl]amino}carbonyl)-4-hydroxy-4-(1-methyl-6-oxo-1,6-dihydropyridin-3-yl)piperidine-1-carboxylate

To a solution of rac-tert-butyl (3S,4R)-3-({cyclopropyl[3-(2-methoxyethoxy)-5-(3-methoxypropyl)benzyl]amino}carbonyl)-4-hydroxy-4-(6-hydroxypyridin-3-yl)piperidine-1-carboxylate from the previous step (1 eq.) in MeOH (0.04M) at 0° C. was added 2M NaOH (3 eq.) followed by dimethylsulfate (4.36 eq.). The reaction was allowed to slowly warm to rt overnight. The methanol was evaporated in vacuo and the aqueous layer quenched with saturated aqueous NH₄Cl. The aqueous phase was then extracted 2× with EtOAc and the combined organic extracts were washed with water, brine, dried (MgSO₄), filtered then concentrated in vacuo. The resulting residue was purified by column chromatography (2-3% (2M NH₃ in MeOH) in CH₂Cl₂) to afford the title compound.

### Step 5: rac-(3S,4R)—N-cyclopropyl-4-hydroxy-N-[3-(2-methoxyethoxy)-5-(3-methoxypropyl)benzyl]-4-(1-methyl-6-oxo-1,6-dihydropyridin-3-yl)piperidine-3-carboxamide

To a solution of rac-tert-butyl (3S,4R)-3-({cyclopropyl[3-(2-methoxyethoxy)-5-(3-methoxypropyl)benzyl]amino}carbonyl)-4-hydroxy-4-(1-methyl-6-oxo-1,6-dihydropyridin-3-yl)piperidine-1-carboxylate (1 eq.) from step 4 in CH₂Cl₂ (0.04 M) at rt was added HCl (4 M in dioxane, 30 eq.). The reaction was stirred at rt for 2 h. The reaction was then concentrated in vacuo, purified by reverse phase HPLC (C₁₈; 5-95% MeCN/water+0.1% TFA) and concentrated in vacuo. The residue was extracted with CH₂Cl₂ from aq. NaHCO₃, dried over Na₂SO₄, filtered and concentrated in vacuo to afford the title compound as a clear colorless oil. MS: ESI+ve 528.0 (MH+).

### Example 25

### rac-(3S,4R)—N-{[1,3-bis(3-methoxypropyl)-2,4-dioxo-1,2,3,4-tetrahydropyrimidin-5-yl]methyl}-N-cyclopropyl-4-(3,4-difluorophenyl)-4-hydroxypiperidine-3-carboxamide

### Step 1: rac-tert-butyl (3S,4R)-3-{[{[1,3-bis(3-methoxypropyl)-2,4-dioxo-1,2,3,4-tetrahydropyrimidin-5-yl]methyl}(cyclopropyl)amino]carbonyl}-4-(3,4-difluorophenyl)-4-hydroxypiperidine-1-carboxylate

Lithium chloride in a round bottom flask was dried under vacuum at 120° C. overnight. It was further flame-dried under vacuum before its use. To lithium chloride (5 eq.) under N₂ was added 3,4-difluorophenylmagnesium bromide (0.5 M in THF, 3 eq.). The mixture was stirred at rt for 15 min then cooled to 0° C. The cooled mixture was then added dropwise to a solution of keto amide 3.2 (1 eq.) in THF (9.0 M) at 0° C. After 15 min, the reaction was quenched with NH₄Cl and extracted 3× with EtOAc. The combined organic extracts were dried (MgSO₄), filtered and concentrated in vacuo. Purification by automated flash chromatography on silica gel (5% MeOH in CH₂Cl₂) afforded the title compound along with some impurities. The residue was resubjected to automated flash chromatography (50-100% EtOAc in hexanes) to afford the title compound.

### Step 2: rac-(3S,4R)—N-{[1,3-bis(3-methoxypropyl)-2,4-dioxo-1,2,3,4-tetrahydropyrimidin-5-yl]methyl}-N-cyclopropyl-4-(3,4-difluorophenyl)-4-hydroxypiperidine-3-carboxamide

To a solution of the title compound from step 1 (1 eq.) in CH₂Cl₂ (0.06 M) at rt was added HCl (4 M in dioxane, 35 eq.). The reaction was stirred at rt for 1 h and concentrated in vacuo. Purification by column chromatography on silica gel (10% (2M NH₃ in MeOH) in CH₂Cl₂) afforded the title compound as a clear colorless oil. MS: ESI+ve 565.1 (MH+).

### Example 26

### rac-(3S,4R)—N-[2-chloro-5-(2-methoxyethyl)benzyl]-N-cyclopropyl-4-(3,4-difluorophenyl)-4-hydroxypiperidine-3-carboxamide

### Step 1: rac-tert-butyl (3S,4R)-3-{[[2-chloro-5-(2-methoxyethyl)benzyl](cyclopropyl)amino]carbonyl}-4-(3,4-difluorophenyl)-4-hydroxypiperidine-1-carboxylate

Lithium chloride in a round bottom flask was dried under vacuum at 120° C. overnight. It was further flame-dried under vacuum before its use. To lithium chloride (3 eq.) under N₂ was added 3,4-fluorophenylmagnesium bromide (0.5 M in THF, 3 eq.). The mixture was stirred at rt for 15 min. The mixture was then added dropwise to a 0° C. solution of keto amide 3.4 (1 eq.) in THF (0.2M). After 15 min at 0° C., the reaction was quenched with NH₄Cl and extracted with Et₂O. The organic extract was dried (MgSO₄), filtered and concentrated in vacuo. Purification by automated flash chromatography on silica gel (20-40% EtOAc in hexanes) afforded the title compound as a white solid.

### Step 2: rac-(3S,4R)—N-[2-chloro-5-(2-methoxyethyl)benzyl]-N-cyclopropyl-4-(3,4-difluorophenyl)-4-hydroxypiperidine-3-carboxamide

To a solution of the title compound from step 1 (1 eq.) in CH₂Cl₂ (0.1 M) was added HCl (4 M in dioxane, 41 eq.). The reaction was stirred at rt for 2.5 h and concentrated in vacuo. The residue was taken up in CH₂Cl₂ and washed with NaOH (1M), brine, dried (Na₂SO₄) filtered and concentrated in vacuo to afford the title compound as a colorless glass. MS: ESI+ve 478.8 (MH+).

### Example 27

### rac-(3S,4R)—N-cyclopropyl-N-(2,3-dichlorobenzyl)-4-(3,4-difluorophenyl)-4-hydroxypiperidine-3-carboxamide

### Step 1: rac-tert-butyl (3S,4R)-3-{[cyclopropyl(2,3-dichlorobenzyl)amino]carbonyl}-4-(3,4-difluorophenyl)-4-hydroxypiperidine-1-carboxylate

Lithium chloride in a round bottom flask was dried under vacuum at 120° C. overnight. It was further flame-dried under vacuum before its use. To lithium chloride (3 eq.) under N₂ was added 3,4-fluorophenylmagnesium bromide (0.5 M in THF, 3 eq.). The mixture was stirred at rt for 15 min. The mixture was then added dropwise to a 0° C. solution of keto amide 3.3 (1 eq.) in THF (0.2M). After 15 min at 0° C., the reaction was quenched with NH₄Cl and extracted with Et₂O. The organic extract was dried (MgSO₄), filtered and concentrated in vacuo. Purification by automated flash chromatography on silica gel (20-40% EtOAc in hexanes) afforded the title compound as a white solid.

### Step 2: rac-(3S,4R)—N-cyclopropyl-N-(2,3-dichlorobenzyl)-4-(3,4-difluorophenyl)-4-hydroxypiperidine-3-carboxamide

To a solution of the title compound from step 1 (1 eq.) in CH₂Cl₂ (0.1 M) at rt was added HCl (4 M in dioxane, 34 eq.). The reaction was stirred at rt for 2.5 h. The reaction was then concentrated in vacuo. The residue was taken up in CH₂Cl₂ and washed with NaOH (1M) then brine, dried (Na₂SO₄) and concentrated in vacuo to give the title compound as a clear oil. MS: ESI+ve 454.9 (MH+).

### Example 28

### rac-(3S,4R)—N-[2-chloro-5-(3-methoxypropyl)benzyl]-N-cyclopropyl-4-(3,4-difluorophenyl)-4-hydroxypiperidine-3-carboxamide

### Step 1: tert-butyl (3S,4R)-3-{[[2-chloro-5-(3-methoxypropyl)benzyl](cyclopropyl)amino]carbonyl}-4-(3,4-difluorophenyl)-4-hydroxypiperidine-1-carboxylate

Lithium chloride in a round bottom flask was dried under vacuum at 120° C. overnight. It was further flame-dried under vacuum before its use. To lithium chloride (9.8 eq.) under N₂ was added 3,4-fluorophenylmagnesium bromide (0.5 M in THF, 2.6 eq.). The mixture was stirred at rt for 3 h upon which all the lithium chloride dissolved. The mixture was then added dropwise to a 0° C. solution of keto amide 3.5 (1 eq.) in THF (0.2M). After 7 min at 0° C., the reaction was quenched with NH₄Cl and extracted with EtOAc. The organic extract was dried (Na₂SO₄), filtered and concentrated in vacuo. Purification by automated flash chromatography on silica gel (10-80% EtOAc in hexanes) afforded the title compound.

### Step 2: rac-(3S,4R)—N-[2-chloro-5-(3-methoxypropyl)benzyl]-N-cyclopropyl-4-(3,4-difluorophenyl)-4-hydroxypiperidine-3-carboxamide

To a solution of the title compound from step 1 (1 eq.) in CH₂Cl₂ (0.1 M) at rt was added HCl (4 M in dioxane, 31 eq.). The reaction was stirred at rt for 1.5 h and concentrated in vacuo. The residue was taken up in CH₂Cl₂ and washed with NaOH (1M) then brine, dried (Na₂SO₄) and concentrated in vacuo to afford the title compound as a pale yellow foam. MS: ESI+ve 493.1 (MH+).

### Example 29

### rac-(3S,4R)—N-{[5-chloro-2-(3-methoxypropyl)pyridin-4-yl]methyl}-N-cyclopropyl-4-(3,4-difluorophenyl)-4-hydroxypiperidine-3-carboxamide

### Step 1: tert-butyl (3S,4R)-3-{[{[5-chloro-2-(3-methoxypropyl)pyridin-4-yl]methyl}(cyclopropyl)amino]carbonyl}-4-(3,4-difluorophenyl)-4-hydroxypiperidine-1-carboxylate

Lithium chloride in a round bottom flask was dried under vacuum at 120° C. overnight. It was further flame-dried under vacuum before its use. To lithium chloride (10.7 eq.) under N₂ was added 3,4-fluorophenylmagnesium bromide (0.5 M in THF, 2.8 eq.). The mixture was stirred at rt for 3 h upon which all the lithium chloride dissolved. The mixture was then added dropwise to a 0° C. solution of keto amide 3.6 (1 eq.) in THF (0.2M). After 7 min at 0° C., the reaction was quenched with NH₄Cl and extracted with EtOAc. The organic extract was dried (Na₂SO₄), filtered and concentrated in vacuo. Purification by automated flash chromatography on silica gel (30-100% EtOAc in hexanes) afforded the title compound.

### Step 2: rac-(3S,4R)—N-{[5-chloro-2-(3-methoxypropyl)pyridin-4-yl]methyl}-N-cyclopropyl-4-(3,4-difluorophenyl)-4-hydroxypiperidine-3-carboxamide

To a solution of the title compound from step 1 (1 eq.) in CH₂Cl₂ (0.1 M) was added HCl (4 M in dioxane, 30 eq.). The reaction was stirred at rt for 1.5 h and concentrated in vacuo. The residue was taken up in CH₂Cl₂ and washed with NaOH (1M) then brine, dried (Na₂SO₄) and concentrated in vacuo to give the title compound as a colorless foam. MS: ESI+ve 494.1 (MH+).

### Example 30

### rac-(3S,4R)—N-{[5-chloro-2-(3-methoxypropyl)-1-oxidopyridin-4-yl]methyl}-N-cyclopropyl-4-(3,4-difluorophenyl)-4-hydroxypiperidine-3-carboxamide

### Step 1: tert-butyl (3S,4R)-3-{[{[5-chloro-2-(3-methoxypropyl)-1-oxidopyridin-4-yl]methyl}(cyclopropyl)amino]carbonyl}-4-(3,4-difluorophenyl)-4-hydroxypiperidine-1-carboxylate

To a solution of the title compound from Example 29, step 1 (1 eq.) in CH₂Cl₂ (0.08 M) was added 3-chloroperoxybenzoic acid (1 eq.). The resulting colorless solution was stiffed at 25° C. for 6 h. The solution was quenched with saturated aqueous Na₂S₂O₃ and washed with 1 N aq. NaOH. The aqueous wash was back-extracted with CH₂Cl₂. The combined organic extracts were washed further with water and brine, dried over Na₂SO₄, filtered and concentrated in vacuo to afford the title compound as a colorless oil.

### Step 2: rac-(3S,4R)—N-{[5-chloro-2-(3-methoxypropyl)-1-oxidopyridin-4-yl]methyl}-N-cyclopropyl-4-(3,4-difluorophenyl)-4-hydroxypiperidine-3-carboxamide

To a solution of the title compound from step 1 (1 eq.) in CH₂Cl₂ (0.01 M) at rt was added HCl (4 M in dioxane, 35 eq.). The reaction was stirred at rt for 1 h and concentrated in vacuo. The residue was taken up in CH₂Cl₂ and washed with NaOH (1M) then brine, dried (Na₂SO₄) and concentrated in vacuo to afford the title compound as a colorless foam. MS: ESI+ve 510.2 (MH+).

### Example 31

### rac-(3S,4R)—N-(5-{[acetyl(methyl)amino]methyl}-2-chlorobenzyl)-N-cyclopropyl-4-(3,4-difluorophenyl)-4-hydroxypiperidine-3-carboxamide

### Step 1: tert-butyl (3S,4R)-3-{[(5-{[acetyl(methyl)amino]methyl}-2-chlorobenzyl)(cyclopropyl)amino]carbonyl}-4-(3,4-difluorophenyl)-4-hydroxypiperidine-1-carboxylate

Lithium chloride in a round bottom flask was dried under vacuum at 120° C. overnight. It was further flame-dried under vacuum before its use. To lithium chloride (12 eq.) under N₂ was added 3,4-fluorophenylmagnesium bromide (0.5 M in THF, 3 eq.). The mixture was stirred at rt for 3 h upon which all the lithium chloride dissolved. The mixture was then added dropwise to a 0° C. solution of keto amide 3.7 (1 eq.) in THF (0.2M). After 7 min at 0° C., the reaction was quenched with NH₄Cl and extracted with EtOAc. The organic extract was dried (Na₂SO₄), filtered and concentrated in vacuo. Purification by automated flash chromatography on silica gel (10-80% EtOAc in hexanes) afforded the title compound.

### Step 2: rac-(3S,4R)—N-(5-{[acetyl(methyl)amino]methyl}-2-chlorobenzyl)-N-cyclopropyl-4-(3,4-difluorophenyl)-4-hydroxypiperidine-3-carboxamide

To a solution of the title compound from step 1 (1 eq.) in CH₂Cl₂ (0.006 M) at rt was added HCl (4 M in dioxane, 23 eq.). The reaction was stirred at rt for 1.5 h and concentrated in vacuo. The residue was taken up in CH₂Cl₂ and washed with NaOH (1M) then brine, dried (Na₂SO₄) and concentrated in vacuo to give the title compound as a colorless foam. MS: ESI+ve 506.1 (MH+).

### Example 32

### rac-(3S,4R)—N-cyclopropyl-4-(3,4-difluorophenyl)-4-hydroxy-N-{[1-(3-methoxypropyl)-1H-indol-3-yl]methyl}piperidine-3-carboxamide

### Step 1: tert-butyl (3S,4R)-3-[(cyclopropyl{[1-(3-methoxypropyl)-1H-indol-3-yl]methyl}amino)carbonyl]-4-(3,4-difluorophenyl)-4-hydroxypiperidine-1-carboxylate

Lithium chloride in a round bottom flask was dried under vacuum at 120° C. overnight. It was further flame-dried under vacuum before its use. To lithium chloride (12 eq.) under N₂ was added 3,4-fluorophenylmagnesium bromide (0.5 M in THF, 3 eq.). The mixture was stirred at rt for 3 h upon which all the lithium chloride dissolved. The mixture was then added dropwise to a 0° C. solution of keto amide 3.8 (1 eq.) in THF (0.2M). After 7 min at 0° C., the reaction was quenched with NH₄Cl and extracted with EtOAc. The organic extract was dried (Na₂SO₄), filtered and concentrated in vacuo. Purification by automated flash chromatography on silica gel (10-80% EtOAc in hexanes) afforded the title compound.

### Step 2: rac-(3S,4R)—N-cyclopropyl-4-(3,4-difluorophenyl)-4-hydroxy-N-{[1-(3-methoxypropyl)-1H-indol-3-yl]methyl}piperidine-3-carboxamide

To a solution of the title compound from step 1 (1 eq.) in CH₂Cl₂ (0.07 M) was added zinc bromide (10 eq.). The resulting suspension was sonicated for 1 min before it was allowed to stir at rt for 18 h, sonicating the reaction periodically. The reaction suspension was quenched with NaOH (1M) then extracted with CH₂Cl₂. The combined organic extracts were dried (Na₂SO₄), filtered and concentrated in vacuo to give an oil. Purification by automated flash chromatography (3-20% (5% NH₄OH in MeOH) in CH₂Cl₂) afforded the title compound as a colorless oil. MS: ESI+ve 498.2 (MH+).

CHIRAL TERTIARY ALCOHOL 6

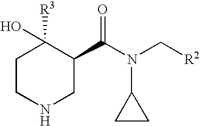

| Ex- | | | |
|---|---|---|---|
| am- | Com- | | |
| ple | pound | R² | R³ |
| 33 | 6.1 | 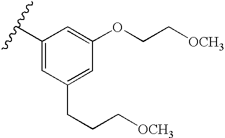 | 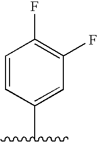 |
| 34 | 6.2 | 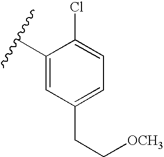 | 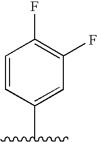 |

### Example 33

### (3S,4R)—N-cyclopropyl-4-(3,4-difluorophenyl)-4-hydroxy-N-[3-(2-methoxyethoxy)-5-(3-methoxypropyl)benzyl]piperidine-3-carboxamide

Rac-(3S,4R)—N-cyclopropyl-4-(3,4-difluorophenyl)-4-hydroxy-N-[3-(2-methoxyethoxy)-5-(3-methoxypropyl)benzyl]piperidine-3-carboxamide (Example 6) (100 mg/mL solution in 40% EtOH in hexanes) was separated by chiral HPLC (Chiralpak AD column; 40% EtOH in hexanes+0.15% Et₃N). The first eluting enantiomer (title compound) was isolated with an er>99:1 as a clear colorless oil. MS: ESI+ve 533.0 (MH+).

### Example 34

### (3S,4R)—N-[2-chloro-5-(3-methoxypropyl)benzyl]-N-cyclopropyl-4-(3,4-difluorophenyl)-4-hydroxypiperidine-3-carboxamide

Rac-(3S,4R)—N-[2-chloro-5-(2-methoxyethyl)benzyl]-N-cyclopropyl-4-(3,4-difluorophenyl)-4-hydroxypiperidine-3-carboxamide (Example 26) was separated by chiral HPLC (Chiralpak AD column; 15% EtOH, 15% iPrOH, 0.25% TEA in hexanes). The second eluting enantiomer (clear colorless oil) was isolated as the title compound. MS: ESI+ve 479.0 (MH+).

RACEMIC TERTIARY ETHER 7

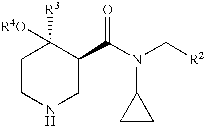

| Example | Compound | R² | R³ | R⁴ |
|---|---|---|---|---|
| 35 QFRET-1 Plasma-4.7 | 7.1 | 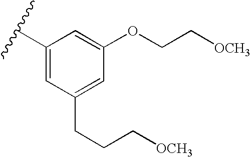 | 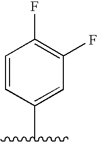 | Me |
| 36 | 7.2 | 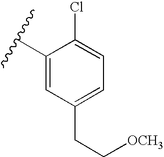 | 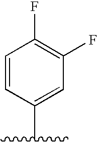 | Me |
| 37 | 7.3 | 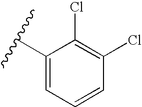 | 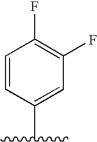 | Me |
| 38 | 7.4 | 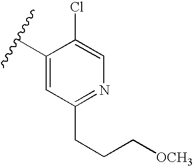 | 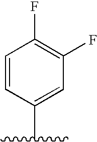 | Me |
| 39 | 7.5 | 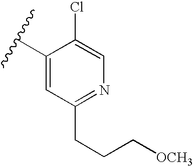 | 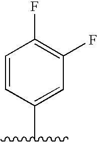 | Me |
| 40 | 7.6 | 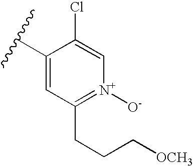 | 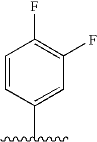 | Me |
| 41 | 7.7 | 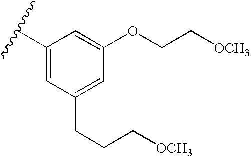 | 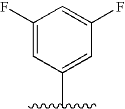 | Me |
| 42 | 7.8 | 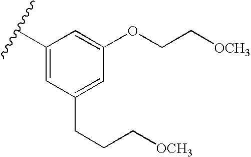 | 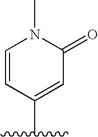 | Me |
| 43 | 7.9 | 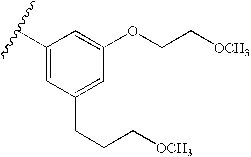 | 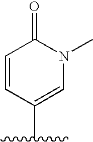 | Me |
| 44 | 7.10 | 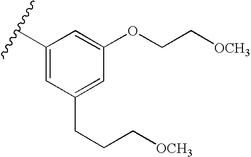 | 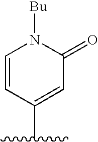 | Me |
| 45 | 7.11 | 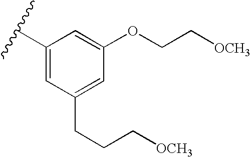 | 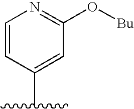 | Me |

### Example 35

### Rac-(3S,4R)—N-cyclopropyl-4-(3,4-difluorophenyl)-4-methoxy-N-[3-(2-methoxyethoxy)-5-(3-methoxypropyl)benzyl]piperidine-3-carboxamide

### Step 1: rac-tert-butyl (3S,4R)-3-({cyclopropyl[3-(2-methoxyethoxy)-5-(3-methoxypropyl)benzyl]amino}carbonyl)-4-(3,4-difluorophenyl)-4-methoxypiperidine-1-carboxylate

To a mixture of the title compound from step 1 of Example 6 (1 eq.) and NaH (1.05 eq) (60% dispersion in oil) was added MeI (10 eq.) and DMF (0.1 M solution). The reaction mixture was heated to 80° C. for 2 h, extracted with 3×Et₂O from water. The organic phase was dried over MgSO₄, filtered and concentrated in vacuo. The residue was purified by flash chromatography (SiO₂; 20-50% EtOAc in hexanes) to afford the title compound as a clear, colorless oil.

### Step 2: rac-(3S,4R)—N-cyclopropyl-4-(3,4-difluorophenyl)-4-methoxy-N-[3-(2-methoxyethoxy)-5-(3-methoxypropyl)benzyl]piperidine-3-carboxamide

A solution of the title compound from step 1 (1 eq.) in HCl (4N in dioxane, 40 eq.) and dichloromethane (twice the volume of HCl) was stirred at rt 30 min and concentrated in vacuo. The residue was purified by flash chromatography (SiO₂; 5-10% (2M NH₃ in MeOH) in CH₂Cl₂) to afford the title compound as a clear colorless oil. MS: ESI+ve 547.2 (MH+).

### Example 36

### Rac-(3S,4R)—N-[2-chloro-5-(2-methoxyethyl)benzyl]-N-cyclopropyl-4-(3,4-difluorophenyl)-4-methoxypiperidine-3-carboxamide

### Step 1: rac-tert-butyl (3S,4R)-3-{[[2-chloro-5-(2-methoxyethyl)benzyl](cyclopropyl)amino]carbonyl}-4-(3,4-difluorophenyl)-4-methoxypiperidine-1-carboxylate

To a mixture of the title compound from step 1 of Example 26 (1 eq.) and NaH (1.05 eq) (60% dispersion in oil) was added MeI (10 eq.) and DMF (0.1 M solution). The reaction mixture was heated to 80° C. for 25 min, and extracted with 3×Et₂O from water. The organic phase was dried over MgSO₄, filtered and concentrated in vacuo. The residue was purified by flash chromatography (SiO₂; 20-50% EtOAc in hexanes) to afford the title compound as a clear, colorless oil.

### Step 2: rac-(3S,4R)—N-[2-chloro-5-(2-methoxyethyl)benzyl]-N-cyclopropyl-4-(3,4-difluorophenyl)-4-methoxypiperidine-3-carboxamide

A solution of the title compound from step 1 (1 eq.) in HCl (4N in dioxane, 59 eq.) and dichloromethane (equal to the volume of HCl) was stirred at rt 2.5 h and concentrated in vacuo. The residue was taken up in dichloromethane and washed with NaOH (aq., 1M) and brine, dried over MgSO₄, filtered and concentrated to afford the title compound as a clear glass. MS: ESI+ve 493.1 (MH+).

### Example 37

### Rac-(3S,4R)—N-cyclopropyl-N-(2,3-dichlorobenzyl)-4-(3,4-difluorophenyl)-4-methoxypiperidine-3-carboxamide

### Step 1: rac-tert-butyl (3S,4R)-3-{[cyclopropyl(2,3-dichlorobenzyl)amino]carbonyl}-4-(3,4-difluorophenyl)-4-methoxypiperidine-1-carboxylate

To a mixture of the title compound from step 1 of Example 27 (1 eq.) and NaH (1.1 eq) (60% dispersion in oil) was added MeI (10 eq.) and DMF (0.1 M solution). The reaction mixture was heated to 80° C. for 25 min, and extracted with 3×Et₂O from water. The organic phase was dried over MgSO₄, filtered and concentrated in vacuo. The residue was purified by flash chromatography (SiO₂; 20-50% EtOAc in hexanes) to afford the title compound as a clear, colorless oil.

### Step 2: rac-(3S,4R)—N-[2-chloro-5-(2-methoxyethyl)benzyl]-N-cyclopropyl-4-(3,4-difluorophenyl)-4-methoxypiperidine-3-carboxamide

A solution of the title compound from step 1 (1 eq.) in HCl (4N in dioxane, 57 eq.) and dichloromethane (equal to the volume of HCl) was stirred at rt 1 h and concentrated in vacuo. The residue was taken up in dichloromethane and washed with NaOH (aq., 1M) and brine, dried over Na₂SO₄, filtered and concentrated in vacuo. The residue was purified by flash chromatography (SiO₂, 0.5/5/95 NH₄OH/MeOH/CH₂Cl₂) to afford the title compound as a clear glass. MS: ESI+ve 469.1 (MH+).

### Example 38

### Rac-(3S,4R)—N-[2-chloro-5-(3-methoxypropyl)benzyl]-N-cyclopropyl-4-(3,4-difluorophenyl)-4-methoxypiperidine-3-carboxamide

### Step 1: rac-tert-butyl (3S,4R)-3-{[[2-chloro-5-(3-methoxypropyl)benzyl](cyclopropyl)amino]carbonyl}-4-(3,4-difluorophenyl)-4-methoxypiperidine-1-carboxylate

A solution of the title compound from step 1 of Example 28 (1 eq.) in DMF (0.056 M solution) at rt was treated with NaH (60% dispersion in oil, 1.1 eq). The mixture was sonicated for 1 min and stirred at rt for 10 min MeI (1.1 eq.) was added and the solution stirred at rt 18 min, quenched with NH₄Cl (aq. sat.) and extracted with Et₂O. The organic phase was washed with water, brine, dried over MgSO₄, filtered and concentrated in vacuo. The residue was purified by flash chromatography (SiO₂; 20-80% EtOAc in hexanes) to afford the title compound as a clear, colorless oil.

### Step 2: rac-(3S,4R)—N-[2-chloro-5-(3-methoxypropyl)benzyl]-N-cyclopropyl-4-(3,4-difluorophenyl)-4-methoxypiperidine-3-carboxamide

A solution of the title compound from step 1 (1 eq.) in HCl (4N in dioxane, 31 eq.) and dichloromethane (equal to the volume of HCl) was stirred at rt 1.5 h and concentrated in vacuo. The residue was taken up in dichloromethane and washed with NaOH (aq., 1M) and brine, dried over Na₂SO₄, filtered and concentrated in vacuo. The residue was purified by flash chromatography (Jan. 9, 1990 NH₄OH/MeOH/CH₂Cl₂) to afford the title compound as a pale yellow foam. MS: ESI+ve 507.2 (MH+).

### Example 39

### Rac-(3S,4R)—N-{[5-chloro-2-(3-methoxypropyl)pyridin-4-yl]methyl}-N-cyclopropyl-4-(3,4-difluorophenyl)-4-methoxypiperidine-3-carboxamide

### Step 1: rac-tert-butyl (3S,4R)-3-{[{[5-chloro-2-(3-methoxypropyl)pyridin-4-yl]methyl}(cyclopropyl)amino]carbonyl}-4-(3,4-difluorophenyl)-4-methoxypiperidine-1-carboxylate

A solution of the title compound from step 1 of Example 29 (1 eq.) in DMF (0.084 M solution) at rt was treated with NaH (60% dispersion in oil, 1.1 eq). The mixture was sonicated for 1 min and stirred at rt for 4 min. MeI (1.1 eq.) was added and the solution stirred at rt 18 min, quenched with NH₄Cl (aq. sat) and extracted with Et₂O. The organic phase was washed with water, brine dried over Na₂SO₄, filtered and concentrated in vacuo. The residue was purified by flash chromatography (SiO₂; 30-100% EtOAc in hexanes) to afford the title compound as a clear, colorless oil.

### Step 2: Rac-(3S,4R)—N-{[5-chloro-2-(3-methoxypropyl)pyridin-4-yl]methyl}-N-cyclopropyl-4-(3,4-difluorophenyl)-4-methoxypiperidine-3-carboxamide

A solution of the title compound from step 1 (1 eq.) in HCl (4N in dioxane, 31 eq.) and dichloromethane (equal to the volume of HCl) was stirred at rt 1.5 h and concentrated in vacuo. The residue was taken up in dichloromethane and washed with NaOH (aq., 1M) and brine, dried over Na₂SO₄, filtered and concentrated in vacuo to afford the title compound as a pale yellow foam. MS: ESI+ve 508.2 (MH+).

### Example 40

### Rac-(3S,4R)—N-{[5-chloro-2-(3-methoxypropyl)-1-oxidopyridin-4-yl]methyl}-N-cyclopropyl-4-(3,4-difluorophenyl)-4-hydroxypiperidine-3-carboxamide

### Step 1: rac-tert-butyl (3S,4R)-3-{[{[5-chloro-2-(3-methoxypropyl)-1-oxidopyridin-4-yl]methyl}(cyclopropyl)amino]carbonyl}-4-(3,4-difluorophenyl)-4-methoxypiperidine-1-carboxylate

To a solution of the title compound from step 1 of Example 39 (1 eq.) in CH₂Cl₂ (0.077 M) was added mCPBA (1.0 eq.). The reaction mixture was stirred at rt for 6 h, quenched with Na₂S₂O₃ (aq. sat.) and washed with NaOH (aq. 1N). The aqueous wash was back-extracted with CH₂Cl₂. The combined organic extracts were washed with water and brine, dried over Na₂SO₄, filtered and concentrated in vacuo, to afford the title compound as a clear, colorless oil.

### Step 2: Rac-(3S,4R)—N-{[5-chloro-2-(3-methoxypropyl)-1-oxidopyridin-4-yl]methyl}-N-cyclopropyl-4-(3,4-difluorophenyl)-4-hydroxypiperidine-3-carboxamide

A solution of the title compound from step 1 (1 eq.) in HCl (4N in dioxane, 36 eq.) and dichloromethane (equal to the volume of HCl) was stirred at rt 1 h and concentrated in vacuo. The residue was taken up in dichloromethane and washed with NaOH (aq., 1M) and brine, dried over Na₂SO₄, filtered and concentrated in vacuo to afford the title compound as a colorless foam. MS: ESI+ve 524.2 (MH+).

### Example 41

### Rac-(3S,4R)—N-cyclopropyl-4-(3,5-difluorophenyl)-4-methoxy-N-[3-(2-methoxyethoxy)-5-(3-methoxypropyl)benzyl]piperidine-3-carboxamide

### Step 1: rac-tert-butyl (3S,4R)-3-({cyclopropyl[3-(2-methoxyethoxy)-5-(3-methoxypropyl)benzyl]amino}carbonyl)-4-(3,5-difluorophenyl)-4-methoxypiperidine-1-carboxylate

To a mixture of the title compound from step 1 of Example 7 (1 eq.) and MeI (10 eq) in DMF (0.1 M solution) was added NaH (60% dispersion in oil, 1.05 eq.). The reaction mixture was heated to 80° C. for 2 h, cooled to rt, taken in Et₂O and washed with 3×H₂O. The organic phase was dried over MgSO₄, filtered and concentrated in vacuo. The residue was purified by flash chromatography (SiO₂; 30-100% EtOAc in hexanes) to afford the title compound as a white solid.

### Step 2: rac-(3S,4R)—N-cyclopropyl-4-(3,5-difluorophenyl)-4-methoxy-N-[3-(2-methoxyethoxy)-5-(3-methoxypropyl)benzyl]piperidine-3-carboxamide

A solution of the title compound from step 1 (1 eq.) in HCl (4N in dioxane, 52 eq.) and dichloromethane (twice the volume of HCl) was stirred at rt 50 min and concentrated in vacuo. The residue was purified by flash chromatography (SiO₂; 5-10% (2M NH₃ in MeOH) in CH₂Cl₂) to afford the title compound as a clear colorless oil. MS: ESI+ve 547.1 (MH+).

### Example 42

### Rac-(3S,4R)—N-cyclopropyl-4-methoxy-N-[3-(2-methoxyethoxy)-5-(3-methoxypropyl)benzyl]-4-(1-methyl-2-oxo-1,2-dihydropyridin-4-yl)piperidine-3-carboxamide

### Step 1: rac-tert-butyl (3S,4R)-3-({cyclopropyl[3-(2-methoxyethoxy)-5-(3-methoxypropyl)benzyl]amino}carbonyl)-4-methoxy-4-(1-methyl-2-oxo-1,2-dihydropyridin-4-yl)piperidine-1-carboxylate

To a solution of the title compound from step 2 of Example 23 (1 eq.) in DMF (0.04 M solution) was added NaH (60% dispersion in oil, 1.2 eq.) then MeI (5 eq). The reaction mixture was heated to 80° C. for 30 min, quenched with water and extracted with 2×Et₂O. The combined organic extracts were washed with brine, dried over MgSO₄, filtered and concentrated in vacuo to afford the title compound as a clear colorless oil.

### Step 2: rac-(3S,4R)—N-cyclopropyl-4-methoxy-N-[3-(2-methoxyethoxy)-5-(3-methoxypropyl)benzyl]-4-(1-methyl-2-oxo-1,2-dihydropyridin-4-yl)piperidine-3-carboxamide

A solution of the title compound from step 1 (1 eq.) in HCl (4N in dioxane, 30 eq.) and dichloromethane (3× the volume of HCl) was stirred at rt 2 h and concentrated in vacuo. The residue was purified by reverse phase HPLC (C₁₈; 5-95% MeCN/water+0.1% TFA) and concentrated in vacuo. The residue was extracted with CH₂Cl₂ from aq. NaHCO₃, dried over Na₂SO₄, filtered and concentrated in vacuo to afford the title compound as a clear colorless oil. MS: ESI+ve 542.6 (MH+).

### Example 43

### Rac-(3S,4R)—N-cyclopropyl-4-hydroxy-N-[3-(2-methoxyethoxy)-5-(3-methoxypropyl)benzyl]-4-(1-methyl-6-oxo-1,6-dihydropyridin-3-yl)piperidine-3-carboxamide

### Step 1: rac-tert-butyl (3S,4R)-3-({cyclopropyl[3-(2-methoxyethoxy)-5-(3-methoxypropyl)benzyl]amino}carbonyl)-4-methoxy-4-(1-methyl-6-oxo-1,6-dihydropyridin-3-yl)piperidine-1-carboxylate

To a solution of the title compound from step 4 of Example 24 (1 eq.) in DMF (0.04 M solution) was added NaH (60% dispersion in oil, 1.2 eq.) then MeI (5 eq). The reaction mixture was heated to 80° C. for 30 min, quenched with water and extracted with 2×Et₂O. The combined organic extracts were washed with brine, dried over MgSO₄, filtered and concentrated in vacuo to afford the title compound as a clear colorless oil.

### Step 2: rac-(3S,4R)—N-cyclopropyl-4-hydroxy-N-[3-(2-methoxyethoxy)-5-(3-methoxypropyl)benzyl]-4-(1-methyl-6-oxo-1,6-dihydropyridin-3-yl)piperidine-3-carboxamide

A solution of the title compound from step 1 (1 eq.) in HCl (4N in dioxane, 30 eq.) and dichloromethane (3× the volume of HCl) was stirred at rt 2 h and concentrated in vacuo. The residue was purified by reverse phase HPLC (C₁₈; 5-95% MeCN/water+0.1% TFA) and concentrated in vacuo. The residue was extracted with CH₂Cl₂ from aq. NaHCO₃, dried over Na₂SO₄, filtered and concentrated in vacuo to afford the title compound as a clear colorless oil. MS: ESI+ve 542.0 (MH+).

### Example 44

### Rac-(3S,4R)-4-(1-butyl-2-oxo-1,2-dihydropyridin-4-yl)-N-cyclopropyl-4-methoxy-N-[3-(2-methoxyethoxy)-5-(3-methoxypropyl)benzyl]piperidine-3-carboxamide

### Step 1: 2-(benzyloxy)-4-bromopyridine

A flame-dried round-bottom flask was charged with 4-bromo-2-pyridinol (1 eq.) benzene (0.14 M solution), Ag₂CO₃ (0.6 eq) and benzyl bromide (1.2 eq.) and heated to 55° C. in the dark for 15 h. The reaction mixture was cooled to rt, filtered through celite, washing with CH₂Cl₂, and the filtrate concentrated in vacuo. The residue was purified by flash chromatography (SiO₂; 2-4% Et₂O in hexanes) to afford the title compound as a clear, colorless oil.

### Step 2: rac-tert-butyl (3S,4R)-4-[2-(benzyloxy)pyridin-4-yl]-3-({cyclopropyl[3-(2-methoxyethoxy)-5-(3-methoxypropyl)benzyl]amino}carbonyl)-4-hydroxypiperidine-1-carboxylate

To a solution of the title compound from step 1 (1.47 eq.) in THF (0.3 M) at −78° C. was added n-BuLi (2.5 M in hexanes, 1.6 eq.). The resulting solution was stirred at −78° C. for 30 min MgBr₂ (0.5 M in Et₂O, 1.9 eq.) was added, and the resulting solution was stirred at −78° C. for 30 min A solution of ketoamide 3.1 (0.1 M in THF, 1 eq.) was added, and the reaction mixture warmed to rt over 16 h, quenched with NaHCO₃ (aq., sat.), extracted with 2×EtOAc. The combined organic phases were washed with brine, dried over MgSO₄, filtered and concentrated in vacuo. The residue was purified by automated flash chromatography (SiO₂; 10-80% EtOAc in hexanes) to afford the title compound as a clear, colorless oil.

### Step 3: rac-tert-butyl (3S,4R)-4-[2-(benzyloxy)pyridin-4-yl]-3-({cyclopropyl[3-(2-methoxyethoxy)-5-(3-methoxypropyl)benzyl]amino}carbonyl)-4-methoxypiperidine-1-carboxylate

To a solution of the title compound from step 2 of (1 eq.) in DMF (0.09 M solution) was added NaH (60% dispersion in oil, 1.4 eq.) then MeI (5 eq). The reaction mixture was heated to 80° C. for 40 min, diluted with Et₂O, washed with 2× water, brine, dried over MgSO₄, filtered and concentrated in vacuo. The residue was purified by automated flash chromatography (0-100% EtOAc in hexanes) to afford the title compound as a clear, colorless oil.

### Step 4: rac-tert-butyl (3S,4R)-3-({cyclopropyl[3-(2-methoxyethoxy)-5-(3-methoxypropyl)benzyl]amino}carbonyl)-4-hydroxy-4-(2-oxo-1,2-dihydropyridin-4-yl)piperidine-1-carboxylate

A solution of the title compound from step 3 (1 eq.) and palladium (10% on carbon) in EtOAc (0.05 M) was stirred under an atmosphere of H₂ for 4 h, filtered through celite, washing with CH₂Cl₂. The filtrate was concentrated in vacuo to afford the title compound as a clear, colorless oil.

### Step 5: rac-tert-butyl (3S,4R)-4-(1-butyl-2-oxo-1,2-dihydropyridin-4-yl)-3-({cyclopropyl[3-(2-methoxyethoxy)-5-(3-methoxypropyl)benzyl]amino}carbonyl)-4-hydroxypiperidine-1-carboxylate

To a solution of the title compound from step 4 (1 eq.) in DMF (0.53 M) was added BuI (3 eq.) and Cs₂CO₃ (1.5 eq.), and the resulting mixture stirred at 60° C. for 16 h. The reaction mixture was extracted with 2×EtOAc from water, dried over MgSO₄, filtered and concentrated in vacuo. The residue was purified by reverse phase HPLC (C₁₈; 5-95% MeCN/water+0.1% TFA) to afford the title compound as a clear colorless oil. Also formed as a by-product is the O-alkylation product, rac-tert-butyl (3S,4R)-4-(2-butoxypyridin-4-yl)-3-({cyclopropyl[3-(2-methoxyethoxy)-5-(3-methoxypropyl)benzyl]amino}carbonyl)-4-hydroxypiperidine-1-carboxylate, isolated as a clear, colorless oil.

### Step 6: rac-(3S,4R)-4-(1-butyl-2-oxo-1,2-dihydropyridin-4-yl)-N-cyclopropyl-4-methoxy-N-[3-(2-methoxyethoxy)-5-(3-methoxypropyl)benzyl]piperidine-3-carboxamide

A solution of the title compound from step 5 (1 eq.) in HCl (4N in dioxane, 30 eq.) and dichloromethane (4.5× the volume of HCl) was stirred at rt 2 h and concentrated in vacuo. The residue was purified by reverse phase HPLC (C₁₈; 5-95% MeCN/water+0.1% TFA) and concentrated in vacuo. The residue was extracted with CH₂Cl₂ from aq. NaHCO₃, dried over Na₂SO₄, filtered and concentrated in vacuo to afford the title compound as a clear colorless oil. MS: ESI+ve 584.3 (MH+).

### Example 45

### Rac-(3S,4R)-4-(2-butoxypyridin-4-yl)-N-cyclopropyl-4-methoxy-N-[3-(2-methoxyethoxy)-5-(3-methoxypropyl)benzyl]piperidine-3-carboxamide

### Step 1: Rac-(3S,4R)-4-(2-butoxypyridin-4-yl)-N-cyclopropyl-4-methoxy-N-[3-(2-methoxyethoxy)-5-(3-methoxypropyl)benzyl]piperidine-3-carboxamide

A solution of the O-alkylation by-product rac-tert-butyl (3S,4R)-4-(2-butoxypyridin-4-yl)-3-({cyclopropyl[3-(2-methoxyethoxy)-5-(3-methoxypropyl)benzyl]amino}carbonyl)-4-hydroxypiperidine-1-carboxylate from Example 44, step 5 (1 eq.) in HCl (4N in dioxane, 30 eq.) and dichloromethane (8× the volume of HCl) was stirred at rt 2 h and concentrated in vacuo. The residue was purified by reverse phase HPLC (C₁₈; 5-95% MeCN/water+0.1% TFA) and concentrated in vacuo. The residue was extracted with CH₂Cl₂ from aq. NaHCO₃, dried over Na₂SO₄, filtered and concentrated in vacuo to afford the title compound as a clear colorless oil. MS: ESI+ve 584.3 (MH+).

CHIRAL TERTIARY ETHER 8

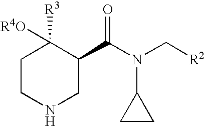

| Example | Compound | R² | R³ | R⁴ |
|---|---|---|---|---|
| 46 | 8.1 | 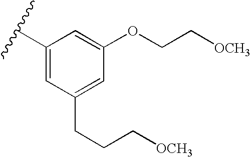 | 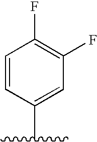 | Me |
| 47 | 8.2 | 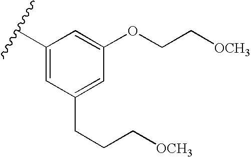 | 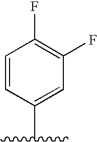 | Et |
| 48 QFRET-1.1 Plasma-61 | 8.3 | 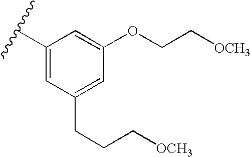 | 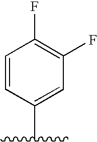 | 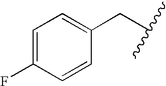 |
| 49 QFRET-2.7 Plasma-11 | 8.4 | 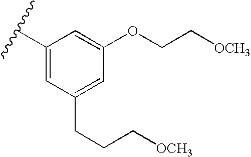 | 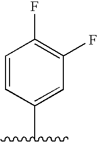 | 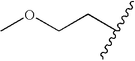 |
| 50 | 8.5 | 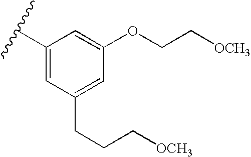 | 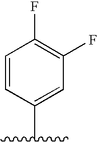 | 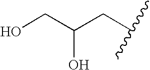 |
| 51 QFRET-3.9 Plasma-21 | 8.6 | 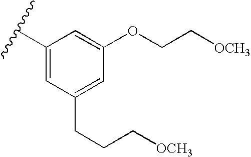 | 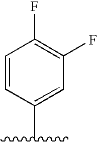 | 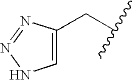 |
| 52 | 8.7 | 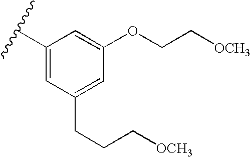 | 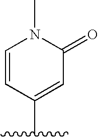 | Me |
| 53 | 8.8 | 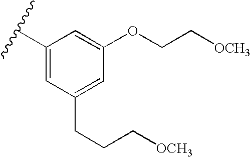 | 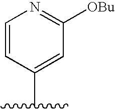 | Me |
| 54 QFRET-2.4 Plasma-4.8 | 8.9 | 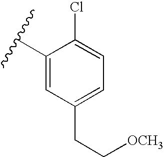 | 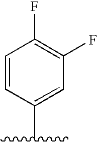 | 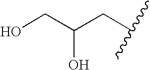 |

### Example 46

### (3S,4R)—N-cyclopropyl-4-(3,4-difluorophenyl)-4-methoxy-N-[3-(2-methoxyethoxy)-5-(3-methoxypropyl)benzyl]piperidine-3-carboxamide

### Step 1: tert-butyl (3S,4R)-3-({cyclopropyl[3-(2-methoxyethoxy)-5-(3-methoxypropyl)benzyl]amino}carbonyl)-4-(3,4-difluorophenyl)-4-hydroxypiperidine-1-carboxylate

To a solution of the title compound from Example 33 (1 eq.) in THF (0.14 M) at rt was added BOC₂O (1.2 eq.). The reaction mixture was stirred at rt 4 h, concentrated in vacuo, and the residue purified by flash chromatography (SiO₂; 10-70% EtOAc in hexanes) to afford the title compound as a white solid.

### Step 2: tert-butyl (3S,4R)-3-({cyclopropyl[3-(2-methoxyethoxy)-5-(3-methoxypropyl)benzyl]amino}carbonyl)-4-(3,4-difluorophenyl)-4-methoxypiperidine-1-carboxylate

To a mixture of the title compound from step 1 (1 eq.) and NaH (1.2 eq) (60% dispersion in oil) was added MeI (5 eq.) and DMF (0.1 M solution). The reaction mixture was heated to 80-90° C. for 40 min after which time another more NaH (1.25 eq.) and MeI (6.2 eq.) were added and heated at the same temperature until there was no further reaction, cooled to rt overnight, and extracted with 2×Et₂O from aq. NH₄Cl. The organic phase was dried over MgSO₄, filtered and concentrated in vacuo. The residue was purified by flash chromatography (SiO₂; 40-50% EtOAc in hexanes) to afford the title compound as a clear, colorless oil.

### Step 3: (3S,4R)—N-cyclopropyl-4-(3,4-difluorophenyl)-4-methoxy-N-[3-(2-methoxyethoxy)-5-(3-methoxypropyl)benzyl]piperidine-3-carboxamide

A solution of the compound from step 2 (1 eq.) in HCl (4N in dioxane, 40 eq.) and dichloromethane (twice the volume of HCl) was stirred at rt 40 min and concentrated in vacuo. The residue was purified by flash chromatography (SiO₂; 5-10% (2M NH3 in MeOH) in CH₂Cl₂) to afford the title compound as a clear colorless oil. MS: ESI+ve 547.5 (MH+).

### Example 47

### (3S,4R)—N-cyclopropyl-4-(3,4-difluorophenyl)-4-ethoxy-N-[3-(2-methoxyethoxy)-5-(3-methoxypropyl)benzyl]piperidine-3-carboxamide

### Step 1: tert-butyl (3S,4R)-3-({cyclopropyl[3-(2-methoxyethoxy)-5-(3-methoxypropyl)benzyl]amino}carbonyl)-4-(3,4-difluorophenyl)-4-ethoxypiperidine-1-carboxylate

To a solution of the title compound from step 1 of Example 46 (1 eq.) and NaH (60% dispersion in oil, 1.2 eq.) in DMF (0.1 M solution) was added ethyl iodide (11 eq.). The solution was heated to 80° C. for 30 min, cooled to rt, and extracted with 2×Et₂O from water. The combined organic extracts were dried over MgSO₄, filtered and concentrated in vacuo. The residue was purified by flash chromatography (SiO₂; 30-50% EtOAc in hexanes) to afford the title compound as a clear, colorless oil.

### Step 2: (3S,4R)—N-cyclopropyl-4-(3,4-difluorophenyl)-4-ethoxy-N-[3-(2-methoxyethoxy)-5-(3-methoxypropyl)benzyl]piperidine-3-carboxamide

A solution of the title compound from step 1 (1 eq.) in HCl (4N in dioxane, 44 eq.) and dichloromethane (twice the volume of HCl) was stirred at rt 1 h and concentrated in vacuo. The residue was purified by flash chromatography (SiO₂; 5-10% (2M NH₃ in MeOH) in CH₂Cl₂) to afford the title compound as a clear colorless oil. MS: ESI+ve 561.7 (MH+).

### Example 48

### (3S,4R)—N-cyclopropyl-4-(3,4-difluorophenyl)-4-[(4-fluorobenzyl)oxy]-N-[3-(2-methoxyethoxy)-5-(3-methoxypropyl)benzyl]piperidine-3-carboxamide

### Step 1: tert-butyl (3S,4R)-3-({cyclopropyl[3-(2-methoxyethoxy)-5-(3-methoxypropyl)benzyl]amino}carbonyl)-4-(3,4-difluorophenyl)-4-[(4-fluorobenzyl)oxy]piperidine-1-carboxylate

To a solution of the title compound from step 1 of Example 46 (1 eq.) and NaH (60% dispersion in oil, 2 eq.) in DMF (0.1 M solution) was added 4-fluorobenzyl bromide (20 eq.). The solution was heated to 80° C. for 1 h, cooled to rt, and extracted with 2×Et₂O from water. The combined organic extracts were dried over MgSO₄, filtered and concentrated in vacuo. The residue was purified by flash chromatography (SiO₂; 30-50% EtOAc in hexanes) to afford the title compound as a clear, colorless oil.

### Step 2: (3S,4R)—N-cyclopropyl-4-(3,4-difluorophenyl)-4-[(4-fluorobenzyl)oxy]-N-[3-(2-methoxyethoxy)-5-(3-methoxypropyl)benzyl]piperidine-3-carboxamide

A solution of the compound from step 1 (1 eq.) in HCl (4N in dioxane, 36 eq.) and dichloromethane (twice the volume of HCl) was stirred at rt 2 h and concentrated in vacuo. The residue was purified by flash chromatography (SiO₂; 5-10% (2M NH₃ in MeOH) in CH₂Cl₂) to afford the title compound as a clear colorless oil. MS: ESI+ve 640.9 (MH+).

### Example 49

### (3S,4R)—N-cyclopropyl-4-(3,4-difluorophenyl)-4-(2-methoxyethoxy)-N-[3-(2-methoxyethoxy)-5-(3-methoxypropyl)benzyl]piperidine-3-carboxamide

### Step 1: tert-butyl (3S,4R)-3-({cyclopropyl[3-(2-methoxyethoxy)-5-(3-methoxypropyl)benzyl]amino}carbonyl)-4-(3,4-difluorophenyl)-4-(2-methoxyethoxy)piperidine-1-carboxylate

To a solution of the title compound from step 1 of Example 46 (1 eq.), NaI (1 eq.) and NaH (60% dispersion in oil, 2 eq.) in DMF (0.1 M solution) was added 1-bromo-2-methoxyethane (11 eq.). The solution was heated to 80° C. for 30 min, cooled to rt, and extracted with 2×Et₂O from water. The combined organic extracts were dried over MgSO₄, filtered and concentrated in vacuo. The residue was purified by flash chromatography (SiO₂; 30-50% EtOAc in hexanes) to afford the title compound as a clear, colorless oil.

### Step 2: (3S,4R)—N-cyclopropyl-4-(3,4-difluorophenyl)-4-(2-methoxyethoxy)-N-[3-(2-methoxyethoxy)-5-(3-methoxypropyl)benzyl]piperidine-3-carboxamide

A solution of the compound from step 1 (1 eq.) in HCl (4N in dioxane, 50 eq.) and dichloromethane (twice the volume of HCl) was stirred at rt 1 h and concentrated in vacuo. The residue was purified by flash chromatography (SiO₂; 5-10% (2M NH₃ in MeOH) in CH₂Cl₂) to afford the title compound as a clear colorless oil. MS: ESI+ve 591.0 (MH+).

### Example 50

### (3S,4R)—N-cyclopropyl-4-(3,4-difluorophenyl)-4-(2,3-dihydroxypropoxy)-N-[3-(2-methoxyethoxy)-5-(3-methoxypropyl)benzyl]piperidine-3-carboxamide trifluoroacetate

### Step 1: tert-butyl (3S,4R)-4-(allyloxy)-3-({cyclopropyl[3-(2-methoxyethoxy)-5-(3-methoxypropyl)benzyl]amino}carbonyl)-4-(3,4-difluorophenyl)piperidine-1-carboxylate

To a solution of the title compound from step 1 of Example 46 (1 eq.) and NaH (60% dispersion in oil, 2 eq.) in DMF (0.095 M solution) was added allyl bromide (14.5 eq.). The solution was heated to 80° C. for 2.5 h, cooled to rt, and extracted with 2×Et₂O from water. The combined organic extracts were dried over MgSO₄, filtered and concentrated in vacuo. The residue was purified by flash chromatography (SiO₂; 20-40% EtOAc in hexanes) to afford the title compound as a clear, colorless oil.

### Step 2: tert-butyl (3S,4R)-3-({cyclopropyl[3-(2-methoxyethoxy)-5-(3-methoxypropyl)benzyl]amino}carbonyl)-4-(3,4-difluorophenyl)-4-(2,3-dihydroxypropoxy)piperidine-1-carboxylate

A mixture of the title compound from step 1 (1 eq.), OsCl₃ (0.01 eq.), quinuclidine (0.05 eq.), K₂CO₃ (3 eq.) and K₃Fe(CN)₆ in a tert-butanol/water mixture (1:1 v/v, 0.14M solution) was stirred at rt overnight. The reaction mixture was extracted with 3×EtOAc from water, and the combined organic extracts concentrated in vacuo (no drying agent was used). The residue was purified by flash chromatography (SiO₂; 80-100% EtOAc in hexanes, then 4% MeOH in EtOAc) to afford the title compound (mixture of diastereomers) as a clear colorless oil.

### Step 3: (3S,4R)—N-cyclopropyl-4-(3,4-difluorophenyl)-4-(2,3-dihydroxypropoxy)-N-[3-(2-methoxyethoxy)-5-(3-methoxypropyl)benzyl]piperidine-3-carboxamide trifluoroacetate

A solution of the compound from step 2 (1 eq.) in HCl (4N in dioxane, 36.5 eq.) and dichloromethane (twice the volume of HCl) was stirred at rt 1 h and concentrated in vacuo. The residue was purified by reverse phase HPLC (C₁₈; 10-90% MeCN in water+1% TFA) to afford the title compound (mixture of diastereomers) as a clear colorless oil. MS: APCI+ve 607.4 (MH+).

### Example 51

### (3S,4R)—N-cyclopropyl-4-(3,4-difluorophenyl)-N-[3-(2-methoxyethoxy)-5-(3-methoxypropyl)benzyl]-4-(1H-1,2,3-triazol-5-ylmethoxy)piperidine-3-carboxamide

### Step 1: tert-butyl (3S,4R)-3-({cyclopropyl[3-(2-methoxyethoxy)-5-(3-methoxypropyl)benzyl]amino}carbonyl)-4-(3,4-difluorophenyl)-4-(prop-2-yn-1-yloxy)piperidine-1-carboxylate

To a solution of compound the title compound from step 1 of Example 46 (1 eq.) and NaH (60% dispersion in oil, 1.4 eq.) in DMF (0.1 M solution) was added propargyl bromide (80% solution in toluene, 5 eq.). The solution was heated to 80° C. for 2 h, cooled to rt, taken in Et₂O, washed twice with water, washed with brine, dried over MgSO₄, filtered and concentrated in vacuo. The residue was purified by flash chromatography (SiO₂; 20-60% EtOAc in hexanes) to afford the title compound as a pale brown oil.

### Step 2: tert-butyl (3S,4R)-3-({cyclopropyl[3-(2-methoxyethoxy)-5-(3-methoxypropyl)benzyl]amino}carbonyl)-4-(3,4-difluorophenyl)-4-(1H-1,2,3-triazol-4-ylmethoxy)piperidine-1-carboxylate

A mixture of the title compound from step 1 (1 eq.), trimethylsilyl azide (3 eq.) and CuI (0.2 eq.) in a mixture of DMF and MeOH (9:1 respectively, 0.15 M solution) was heated to 100° C. 16 h, cooled to rt, diluted with EtOAc, washed with water and brine, dried over MgSO₄, filtered and concentrated in vacuo. The residue was purified by flash chromatography (SiO₂; 5-8% (2M NH₃ in MeOH) in CH₂Cl₂) to afford the title compound as a dark green oil.

### Step 3: (3S,4R)—N-cyclopropyl-4-(3,4-difluorophenyl)-N-[3-(2-methoxyethoxy)-5-(3-methoxypropyl)benzyl]-4-(1H-1,2,3-triazol-5-ylmethoxy)piperidine-3-carboxamide

A solution of the title compound from step 2 (1 eq.) in HCl (4N in dioxane, 40 eq.) and dichloromethane (twice the volume of HCl) was stirred at rt 2 h and concentrated in vacuo. The residue was purified by flash chromatography (SiO₂; 10% (2M NH₃ in MeOH) in CH₂Cl₂) to afford the title compound as a clear colorless oil. MS: ESI+ve 614.2 (MH+).

### Example 52

### (3S,4R)—N-cyclopropyl-4-methoxy-N-[3-(2-methoxyethoxy)-5-(3-methoxypropyl)benzyl]-4-(1-methyl-2-oxo-1,2-dihydropyridin-4-yl)piperidine-3-carboxamide

### Step 1: tert-butyl (3S,4R)-4-[2-(benzyloxy)pyridin-4-yl]-3-({cyclopropyl[3-(2-methoxyethoxy)-5-(3-methoxypropyl)benzyl]amino}carbonyl)-4-hydroxypiperidine-1-carboxylate

The title compound from Example 22, step 2 was resolved by chiral HPLC (Chiralpak AD; 40% EtOH in hexanes) to afford the title compound (first eluting enantiomer) as a clear colorless oil.

### Step 2: tert-butyl (3S,4R)-4-[2-(benzyloxy)pyridin-4-yl]-3-({cyclopropyl[3-(2-methoxyethoxy)-5-(3-methoxypropyl)benzyl]amino}carbonyl)-4-methoxypiperidine-1-carboxylate

To a solution of the title compound from step 1 (1 eq.) in DMF (0.14 M) at rt was added MeI (1.2 eq.) and NaH (1.2 eq.). The reaction mixture was stirred at rt 30 min, diluted with EtOAc, washed with 4× water, once with brine, dried over MgSO₄, filtered and concentrated in vacuo, to afford the title compound as a clear colorless oil.

### Step 3: tert-butyl (3S,4R)-3-({cyclopropyl[3-(2-methoxyethoxy)-5-(3-methoxypropyl)benzyl]amino}carbonyl)-4-hydroxy-4-(2-oxo-1,2-dihydropyridin-4-yl)piperidine-1-carboxylate

A mixture of the title compound from step 2 (1 eq.), acetic acid (1 eq.) and Pd (10% on carbon) in EtOAc (0.1 M) was stirred at rt under an atmosphere of H₂ for 5 h. The reaction mixture was filtered through celite, washing with EtOAc. The filtrate was concentrated in vacuo to afford the title compound.

### Step 4: tert-butyl (3S,4R)-3-({cyclopropyl[3-(2-methoxyethoxy)-5-(3-methoxypropyl)benzyl]amino}carbonyl)-4-methoxy-4-(1-methyl-2-oxo-1,2-dihydropyridin-4-yl)piperidine-1-carboxylate

To a solution of the title compound from step 3 (1 eq.) in MeOH (0.05 M) at 0° C. was added NaOH (aq., 3M, 3 eq.), followed by Me₂SO₄ (4.4 eq.). The reaction mixture was allowed to warm slowly to rt over 16 h, and concentrated in vacuo. The residue was purified by flash chromatography (SiO₂; 5% (2M NH₃ in MeOH) in CH₂Cl₂) to afford the title compound as a clear colorless oil.

### Step 5: (3S,4R)—N-cyclopropyl-4-methoxy-N-[3-(2-methoxyethoxy)-5-(3-methoxypropyl)benzyl]-4-(1-methyl-2-oxo-1,2-dihydropyridin-4-yl)piperidine-3-carboxamide

A solution of the title compound from step 4 (1 eq.) in HCl (4 N in dioxane, 15 eq.) and CH₂Cl₂ (2.5× the volume of HCl) was stirred at rt for 2 h. The residue was concentrated in vacuo and the residue purified by flash chromatography (SiO₂; 5% (2M NH₃ in MeOH) in CH₂Cl₂) to afford the title compound as a clear colorless oil. MS: APCI+ve 542.3 (MH+).

### Example 53

### (3S,4R)-4-(2-butoxypyridin-4-yl)-N-cyclopropyl-4-methoxy-N-[3-(2-methoxyethoxy)-5-(3-methoxypropyl)benzyl]piperidine-3-carboxamide

### Step 1: tert-butyl (3S,4R)-4-(2-butoxypyridin-4-yl)-3-({cyclopropyl[3-(2-methoxyethoxy)-5-(3-methoxypropyl)benzyl]amino}carbonyl)-4-methoxypiperidine-1-carboxylate

To a solution of the title compound from Example 52, step 3 (1 eq.) in benzene (0.1 M solution) was added 1-iodobutane (1.2 eq.) and Ag₂CO₃ (0.6 eq.). The reaction mixture was stirred at 50° C. in the dark overnight, cooled to rt, filtered through celite, washing with CH₂Cl₂, and concentrated in vacuo. The residue was resubmitted to the reaction conditions, then worked-up identically. The residue was purified by flash chromatography (SiO₂; 60% EtOAc in hexanes) to afford the title compound as a clear colorless oil.

### Step 2: (3S,4R)-4-(2-butoxypyridin-4-yl)-N-cyclopropyl-4-methoxy-N-[3-(2-methoxyethoxy)-5-(3-methoxypropyl)benzyl]piperidine-3-carboxamide

A solution of the title compound from step 4 (1 eq.) in HCl (4 N in dioxane, 15 eq.) and CH₂Cl₂ (2.5× the volume of HCl) was stirred at rt for 5 h. The residue was concentrated in vacuo and the residue purified by flash chromatography (SiO₂; 5% (2M NH₃ in MeOH) in CH₂Cl₂) to afford the title compound as a clear colorless oil. MS: APCI+ve 584.2 (MH+).

### Example 54

### (3S,4R)—N-[2-chloro-5-(2-methoxyethyl)benzyl]-N-cyclopropyl-4-(3,4-difluorophenyl)-4-(2,3-dihydroxypropoxy)piperidine-3-carboxamide

### Step 1: tert-butyl (3S,4R)-3-{[[2-chloro-5-(2-methoxyethyl)benzyl](cyclopropyl)amino]carbonyl}-4-(3,4-difluorophenyl)-4-hydroxypiperidine-1-carboxylate

To a solution of the title compound from Example 34 (1 eq.) in THF (0.1 M solution) at rt was added (BOC)₂O (1.2 eq.) in THF (one-half the volume used to dissolve the title compound from Example 34). The reaction mixture was stirred at rt 1 h, concentrated in vacuo, and the residue purified by automated flash chromatography (SiO₂; 10-50% EtOAc in hexanes) to afford the title compound as a clear glass.

### Step 2: tert-butyl (3S,4R)-4-(allyloxy)-3-{[[2-chloro-5-(2-methoxyethyl)benzyl](cyclopropyl)amino]carbonyl}-4-(3,4-difluorophenyl)piperidine-1-carboxylate

To a solution of the title compound from step 1 (1 eq.) and allyl bromide (3 eq.) in DMF (0.1 M solution) at rt was added NaH (2 eq.). The solution was stiffed at rt 8 min, and extracted with 2×Et₂O from water. The combined organic extracts were dried over MgSO₄, filtered and concentrated in vacuo. The residue was purified by automated flash chromatography (SiO₂; 10-50% EtOAc in hexanes) to afford the title compound as a pale yellow oil.

### Step 3: tert-butyl (3S,4R)-3-{[[2-chloro-5-(2-methoxyethyl)benzyl](cyclopropyl)amino]carbonyl}-4-(3,4-difluorophenyl)-4-(2,3-dihydroxypropoxy)piperidine-1-carboxylate

The title compound from step 2 (1 eq.) was taken in a 1:1 mixture of tert-butanol and water (0.1 M) and cooled to 0° C. and AD-mix- (1 eq.) was added. The resulting mixture was stirred at 0° C. for 4 h, quenched with Na₂S₂O₃ (aq. sat.), extracted with Et₂O, dried over Na₂SO₄, filtered and concentrated in vacuo. The residue was purified by flash chromatography (SiO₂; 2-3% MeOH in EtOAc) to afford the title compound as a clear colorless oil.

### Step 4: (3S,4R)—N-[2-chloro-5-(2-methoxyethyl)benzyl]-N-cyclopropyl-4-(3,4-difluorophenyl)-4-(2,3-dihydroxypropoxy)piperidine-3-carboxamide

A solution of the title compound from step 3 (1 eq.) in HCl (4N in dioxane, 29 eq.) and dichloromethane (equal to the volume of HCl) was stirred at rt 2 h and concentrated in vacuo. The residue was taken up in dichloromethane and washed with NaOH (aq., 1M) and brine, dried over Na₂SO₄, filtered and concentrated to afford the title compound as a clear glass. MS: ESI+ve 553.1 (MH+).

### Assays Demonstrating Biological Activity

### Inhibition of Human Recombinant Renin

The enzymatic in vitro assay was performed in 384-well polypropylene plates (Nunc). The assay buffer consisted of PBS (Gibco BRL) including 1 mM EDTA and 0.1% BSA. The reaction mixture were composed of 47.5 μL per well of an enzyme mix and 2.5 μL of renin inhibitors in DMSO. The enzyme mix was premixed at 4° C. and consists of the following components:

human recombinant renin (40 pM)

synthetic human angiotensin(1-14) (0.5 μM)

hydroxyquinoline sulfate (1 mM)

The mixtures were then incubated at 37° C. for 3 h. The enzyme reaction was stopped by placing the reaction plate on wet ice.

To determine the enzymatic activity and its inhibition, the accumulated Ang I was detected by an enzyme immunoassay (EIA) in 384-well plates (Nunc). 5 μL of the reaction mixture or standards were transferred to immuno plates which were previously coated with a covalent complex of Ang I and bovine serum albumin (Ang I—BSA). 75 μL of Ang I-antibodies in assay buffer above including 0.01% Tween 20 were added and the plates were incubated at 4° C. overnight.

An alternative protocol could be used by stopping the enzymatic reaction with 0.02N final concentration of HCl. 5 μL of the reaction mixture or standards were transferred to immuno plates and 75 μL of Ang I-antibodies in assay buffer above including 0.01% Tween 20 were added and the plates were incubate at RT for 4 h.

The plates were washed 3 times with PBS including 0.01% Tween 20, and then incubated for 2 h at RT with an anti rabbit-peroxidase coupled antibody (WA 934, Amersham). After washing the plates 3 times, the peroxidase substrate ABTS ((2,2′-Azino-bis(3-ethylbenzthiazoline-6-sulfonic Acid).2NH₃) was added and the plates incubated for 60 min at RT. The plate was evaluated in a microplate reader at 405 nm. The percentage of inhibition was calculated for each concentration point and the concentration of renin inhibition was determined that inhibited the enzyme activity by 50% (IC₅₀).

### Inhibition of Renin in Human Plasma

The enzymatic in vitro assay was performed in 384-well polypropylene plates (Nunc). The assay buffer consisted of PBS (Gibco BRL) including 1 mM EDTA and 0.1% BSA. The reaction mixture was composed of 80 μL per well of human plasma, enzyme, Ang I-antibodies mix and 5 μL of renin inhibitors in DMSO. The human plasma mix was premixed at 4° C. and consists of

human plasma from 10 normal donors

human recombinant renin (3 pM)

Ang I-antibodies.

The mixtures were then incubated at 37° C. for 2 h.

To determine the enzymatic activity and its inhibition, the accumulated Ang I was detected by an enzyme immunoassay (EIA) in 384-well plates (Nunc). 10 μL of the reaction mixture or standards were transferred to immuno plates which were previously coated with a covalent complex of Ang I and bovine serum albumin (Ang I—BSA). 70 μL assay buffer were added and the plates were incubated at 4° C. overnight. The plates were washed 3 times with PBS including 0.01% Tween 20, and then incubated for 2 h at RT with an anti rabbit-peroxidase coupled antibody (WA 934, Amersham). After washing the plates 3 times, the peroxidase substrate ABTS ((2,2′-Azino-bis(3-ethylbenzthiazoline-6-sulfonic Acid).2NH₃) was added and the plates incubated for 60 min at RT. The plate was evaluated in a microplate reader at 405 nm In vivo animal model—Female double transgenic rats were purchased from RCC Ltd, Füllingsdorf, Switzerland. All animals were maintained under identical conditions and had free access to normal pelleted rat chow and water. Rats were initially treated with enalapril (1 mg/kg/day) during 2 months. After approximately two weeks following cessation of enalapril treatment the double transgenic rats become hypertensive and reach mean arterial blood pressures in the range of 160-170 mmHg

Transmitter implantation—The rats were anaesthetised with a mixture of 90 mg/kg Ketamin-HCl (Ketavet, Parke-Davis, Berlin FRG) and 10 mg/kg xylazin (Rompun, Bayer, Leverkusen, FRG) i.p. The pressure transmitter was implanted under aseptic conditions into the peritoneal cavity with the sensing catheter placed in the descending aorta below the renal arteries pointing upstream. The transmitter was sutured to the abdominal musculature and the skin closed.

Telemetry-System—Telemetry units were obtained from Data Sciences (St. Paul, Minn.). The implanted sensor consisted of a fluid-filled catheter (0.7 mm diameter, 8 cm long; model TA11PA-C40) connected to a highly stable low-conductance strain-gauge pressure transducer, which measured the absolute arterial pressure relative to a vacuum, and a radio-frequency transmitter. The tip of the catheter was filled with a viscous gel that prevents blood reflux and was coated with an antithrombogenic film to inhibit thrombus formation. The implants (length=2.5 cm, diameter=1.2 cm) weighted 9 g and have a typical battery life of 6 months. A receiver platform (RPC-1, Data Sciences) connected the radio signal to digitized input that was sent to a dedicated personal computer (Compaq, deskpro). Arterial pressures were calibrated by using an input from an ambient-pressure reference (APR-1, Data Sciences). Systolic, mean and diastolic blood pressure was expressed in millimeter of mercury (mmHg).

Hemodynamic measurements—Double transgenic rats with implanted pressure transmitters were dosed by oral gavage with vehicle or 10 mg/kg of the test substance (n=6 per group) and the mean arterial blood pressure was continuously monitored. The effect of the test substance is expressed as maximal decrease of mean arterial pressure (MAP) in the treated group versus the control group.

What is claimed is:

1. A compound of formula I, or a pharmaceutically acceptable salt thereof, having the formula (I) 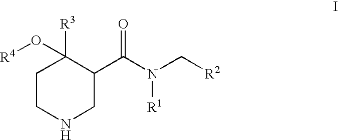
wherein
R¹ is selected from the group consisting of C₁₋₆alkyl or C₃₋₈cycloalkyl;
R² is an aryl ring, a 5 or 6-membered heteroaryl ring containing 1, 2, 3 or 4 heteroatoms selected from N, O or S, or a fused 9 or 10-membered heteroaryl ring system containing 1, 2, 3 or 4 heteroatoms selected from N, O or S, wherein said aryl or heteroaryl ring is unsubstituted or mono-, di-, tri- or tetra-substituted with a group independently selected from
1) halogen,
2) O—C₁₋₅alkylene-O—C₁₋₅alkyl,
3) C₁₋₅alkylene-O—C₁₋₅alkyl,
4) C₁₋₅alkylene-N(C₁₋₅alkyl)-C(O)—C₁₋₅alkyl,
5) C₁₋₅alkylene-NH—C(O)—C₁₋₅alkyl, and
6) oxo;
R³ is an aryl ring, a 5 or 6-membered heteroaryl ring containing 1, 2, 3 or 4 heteroatoms selected from N, O or S, a fused 9 or 10-membered heteroaryl ring system containing 1, 2, 3 or 4 heteroatoms selected from N, O or S, or C₃₋₈ cycloalkyl, wherein said aryl ring, heteroaryl ring, or C₃₋₈ cycloalkyl is unsubstituted or mono-, di-, tri- or tetra-substituted with a group independently selected from
1) halogen,
2) C₁₋₅alkoxy,
3) CF₃,
4) NH₂,
5) O—(C₁₋₅alkylene)-aryl,
6) C₁₋₅ alkyl, and
7) oxo,
R⁴ is selected from the group consisting of
hydrogen,
C₁₋₅alkyl,
C₁₋₅alkylene-aryl,
C₁₋₅allylene-O—C₁₋₅alkyl,
C₃₋₈cycloalkyl,
C₁₋₅alkyleneNHC(O)—C₁₋₅alkyl,
C(O)—O—C₁₋₅alkyl, and
C₁₋₅alkylene-heteroaryl,
wherein aryl is unsubstituted or mono- or di-substituted with halogen, alkyl is unsubstituted or mono- or di-substituted with OH, and heteroaryl is a 5 or 6 membered unsaturated ring containing 1, 2, 3 or 4 heteroatoms selected from the group consisting of N, O and S.

2. A compound of claim 1, or a pharmaceutically acceptable salt thereof,
wherein R¹ is cyclopropyl.

3. A compound of claim 1, or a pharmaceutically acceptable salt thereof,
wherein
R² is phenyl, pyridine, pyrimidine or indole, unsubstituted or mono-, di-, tri- or tetra-substituted with a group independently selected from
1) Cl,
2) O(CH₂)₂OCH₃,
3) (CH₂)₂₋₃OCH₃,
4) CH₂N(CH₃)C(O)CH₃, and
5) oxo.

4. A compound of claim 1, or a pharmaceutically acceptable salt thereof,
wherein
R³ is phenyl, pyridinyl, thiazole, imidazole or benzoxazole, unsubstituted or mono-, di-, tri- or tetra-substituted with a group independently selected from
1) Cl,
2) F,
3) C₁₋₄alkoxy,
4) CF₃,
5) NH₂,
6) OCH₂phenyl,
7) C₁₋₄ alkyl, and
8) oxo.

5. A compound of claim 1, or a pharmaceutically acceptable salt thereof,
wherein R⁴ is selected from the group consisting of
hydrogen,
C₁₋₅allyl,
CH₂fluorophenyl,
(CH₂)₂OCH₃,
CH₂CH(OH)CH₂OH, and
CH₂triazole.

6. A compound of claim 1, or a pharmaceutically acceptable salt thereof,
having the diastereomeric structure

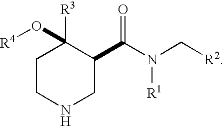

7. A compound of claim 1, or a pharmaceutically acceptable salt thereof,
having the enantiomeric structure

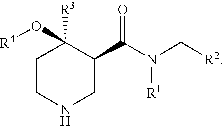

8. A compound of claim 1, or a pharmaceutically acceptable salt thereof,
selected from the group consisting of
rac-(3S,4R)—N-cyclopropyl-4-hydroxy-N-[3-(2-methoxyethoxy)-5-(3-methoxypropyl)benzyl]-4-phenylpiperidine-3-carboxamide,
rac-(3S,4R)—N-cyclopropyl-4-hydroxy-N-[3-(2-methoxyethoxy)-5-(3-methoxypropyl)benzyl]-4-pyridin-3-ylpiperidine-3-carboxamide,
rac-(3S,4R)—N-cyclopropyl-4-hydroxy-N-[3-(2-methoxyethoxy)-5-(3-methoxypropyl)benzyl]-4-pyridin-4-ylpiperidine-3-carboxamide,
rac-(3S,4R)—N-cyclopropyl-4-(4-fluorophenyl)-4-hydroxy-N-[3-(2-methoxyethoxy)-5-(3-methoxypropyl)benzyl]piperidine-3-carboxamide,
rac-(3S,4R)—N-cyclopropyl-4-(3-fluorophenyl)-4-hydroxy-N-[3-(2-methoxyethoxy)-5-(3-methoxypropyl)benzyl]piperidine-3-carboxamide,
rac-(3S,4R)—N-cyclopropyl-4-(3,4-difluorophenyl)-4-hydroxy-N-[3-(2-methoxyethoxy)-5-(3-methoxypropyl)benzyl]piperidine-3-carboxamide,
rac-(3S,4R)—N-cyclopropyl-4-(3,5-difluorophenyl)-4-hydroxy-N-[3-(2-methoxyethoxy)-5-(3-methoxypropyl)benzyl]piperidine-3-carboxamide,
rac-(3S,4R)-4-(3-chlorophenyl)-N-cyclopropyl-4-hydroxy-N-[3-(2-methoxyethoxy)-5-(3-methoxypropyl)benzyl]piperidine-3-carboxamide,
rac-(3S,4R)-4-(4-chlorophenyl)-N-cyclopropyl-4-hydroxy-N-[3-(2-methoxyethoxy)-5-(3-methoxypropyl)benzyl]piperidine-3-carboxamide,
rac-(3S,4R)-4-(4-chloro-3-fluorophenyl)-N-cyclopropyl-4-hydroxy-N-[3-(2-methoxyethoxy)-5-(3-methoxypropyl)benzyl]piperidine-3-carboxamide,
rac-(3S,4R)—N-cyclopropyl-4-(3,4-dichlorophenyl)-4-hydroxy-N-[3-(2-methoxyethoxy)-5-(3-methoxypropyl)benzyl]piperidine-3-carboxamide,
rac-(3S,4R)—N-cyclopropyl-4-hydroxy-N-[3-(2-methoxyethoxy)-5-(3-methoxypropyl)benzyl]-4-(2-methoxyphenyl)piperidine-3-carboxamide,
rac-(3S,4R)-4-[4-chloro-3-(trifluoromethyl)phenyl]-N-cyclopropyl-4-hydroxy-N-[3-(2-methoxyethoxy)-5-(3-methoxypropyl)benzyl]piperidine-3-carboxamide,
rac-(3S,4R)—N-cyclopropyl-4-[2-fluoro-4-(trifluoromethyl)phenyl]-4-hydroxy-N-[3-(2-methoxyethoxy)-5-(3-methoxypropyl)benzyl]piperidine-3-carboxamide,
rac-(3S,4R)—N-cyclopropyl-4-hydroxy-N-[3-(2-methoxyethoxy)-5-(3-methoxypropyl)benzyl]-4-(3-methoxyphenyl)piperidine-3-carboxamide,
rac-(3S,4R)-4-(3-aminophenyl)-N-cyclopropyl-4-hydroxy-N-[3-(2-methoxyethoxy)-5-(3-methoxypropyl)benzyl]piperidine-3-carboxamide,
rac-(3S,4R)—N-cyclopropyl-4-hydroxy-N-[3-(2-methoxyethoxy)-5-(3-methoxypropyl)benzyl]-4-(1,3-thiazol-2-yl)piperidine-3-carboxamide,
rac-(3S,4R)—N-cyclopropyl-4-hydroxy-N-[3-(2-methoxyethoxy)-5-(3-methoxypropyl)benzyl]-4-(1-methyl-1H-imidazol-2-yl)piperidine-3-carboxamide,
rac-(3S,4R)—N-cyclopropyl-4-hydroxy-N-[3-(2-methoxyethoxy)-5-(3-methoxypropyl)benzyl]-4-(1H-1,2,3-triazol-4-yl)piperidine-3-carboxamide,
rac-(3S,4R)—N-cyclopropyl-4-hydroxy-N-[3-(2-methoxyethoxy)-5-(3-methoxypropyl)benzyl]-4-(2-thienyl)piperidine-3-carboxamide,
rac-(3S,4R)-4-(1,3-benzoxazol-2-yl)-N-cyclopropyl-4-hydroxy-N-[3-(2-methoxyethoxy)-5-(3-methoxypropyl)benzyl]piperidine-3-carboxamide,
rac-(3S,4R)-4-[2-(benzyloxy)pyridin-4-yl]-N-cyclopropyl-4-hydroxy-N-[3-(2-methoxyethoxy)-5-(3-methoxypropyl)benzyl]piperidine-3-carboxamide,
rac-(3S,4R)—N-cyclopropyl-4-hydroxy-N-[3-(2-methoxyethoxy)-5-(3-methoxypropyl)benzyl]-4-(1-methyl-2-oxo-1,2-dihydropyridin-4-yl)piperidine-3-carboxamide,
rac-(3S,4R)—N-cyclopropyl-4-hydroxy-N-[3-(2-methoxyethoxy)-5-(3-methoxypropyl)benzyl]-4-(1-methyl-6-oxo-1,6-dihydropyridin-3-yl)piperidine-3-carboxamide,
rac-(3S,4R)—N-{[1,3-bis(3-methoxypropyl)-2,4-dioxo-1,2,3,4-tetrahydropyrimidin-5-yl]methyl}-N-cyclopropyl-4-(3,4-difluorophenyl)-4-hydroxypiperidine-3-carboxamide,
rac-(3S,4R)—N-[2-chloro-5-(2-methoxyethyl)benzyl]-N-cyclopropyl-4-(3,4-difluorophenyl)-4-hydroxypiperidine-3-carboxamide,
rac-(3S,4R)—N-cyclopropyl-N-(2,3-dichlorobenzyl)-4-(3,4-difluorophenyl)-4-hydroxypiperidine-3-carboxamide,
rac-(3S,4R)—N-[2-chloro-5-(3-methoxypropyl)benzyl]-N-cyclopropyl-4-(3,4-difluorophenyl)-4-hydroxypiperidine-3-carboxamide,
rac-(3S,4R)—N-{[5-chloro-2-(3-methoxypropyl)pyridin-4-yl]methyl}-N-cyclopropyl-4-(3,4-difluorophenyl)-4-hydroxypiperidine-3-carboxamide,
rac-(3S,4R)—N-{[5-chloro-2-(3-methoxypropyl)-1-oxidopyridin-4-yl]methyl}-N-cyclopropyl-4-(3,4-difluorophenyl)-4-hydroxypiperidine-3-carboxamide,
rac-(3S,4R)—N-(5-{[acetyl(methyl)amino]methyl}-2-chlorobenzyl)-N-cyclopropyl-4-(3,4-difluorophenyl)-4-hydroxypiperidine-3-carboxamide,
rac-(3S,4R)—N-cyclopropyl-4-(3,4-difluorophenyl)-4-hydroxy-N-{[1-(3-methoxypropyl)-1H-indol-3-yl]methyl}piperidine-3-carboxamide,
(3S,4R)—N-cyclopropyl-4-(3,4-difluorophenyl)-4-hydroxy-N-[3-(2-methoxyethoxy)-5-(3-methoxypropyl)benzyl]piperidine-3-carboxamide,
(3S,4R)—N-[2-chloro-5-(3-methoxypropyl)benzyl]-N-cyclopropyl-4-(3,4-difluorophenyl)-4-hydroxypiperidine-3-carboxamide,
Rac-(3S,4R)—N-cyclopropyl-4-(3,4-difluorophenyl)-4-methoxy-N-[3-(2-methoxyethoxy)-5-(3-methoxypropyl)benzyl]piperidine-3-carboxamide,
Rac-(3S,4R)—N-[2-chloro-5-(2-methoxyethyl)benzyl]-N-cyclopropyl-4-(3,4-difluorophenyl)-4-methoxypiperidine-3-carboxamide,
Rac-(3S,4R)—N-cyclopropyl-N-(2,3-dichlorobenzyl)-4-(3,4-difluorophenyl)-4-methoxypiperidine-3-carboxamide,
Rac-(3S,4R)—N-[2-chloro-5-(3-methoxypropyl)benzyl]-N-cyclopropyl-4-(3,4-difluorophenyl)-4-methoxypiperidine-3-carboxamide,
Rac-(3S,4R)—N-{[5-chloro-2-(3-methoxypropyl)pyridin-4-yl]methyl}-N-cyclopropyl-4-(3,4-difluorophenyl)-4-methoxypiperidine-3-carboxamide,
Rac-(3S,4R)—N-{[5-chloro-2-(3-methoxypropyl)-1-oxidopyridin-4-yl]methyl}-N-cyclopropyl-4-(3,4-difluorophenyl)-4-hydroxypiperidine-3-carboxamide,
Rac-(3S,4R)—N-cyclopropyl-4-(3,5-difluorophenyl)-4-methoxy-N-[3-(2-methoxyethoxy)-5-(3-methoxypropyl)benzyl]piperidine-3-carboxamide,
Rac-(3S,4R)—N-cyclopropyl-4-methoxy-N-[3-(2-methoxyethoxy)-5-(3-methoxypropyl)benzyl]-4-(1-methyl-2-oxo-1,2-dihydropyridin-4-yl)piperidine-3-carboxamide,
Rac-(3S,4R)—N-cyclopropyl-4-hydroxy-N-[3-(2-methoxyethoxy)-5-(3-methoxypropyl)benzyl]-4-(1-methyl-6-oxo-1,6-dihydropyridin-3-yl)piperidine-3-carboxamide,
Rac-(3S,4R)-4-(1-butyl-2-oxo-1,2-dihydropyridin-4-yl)-N-cyclopropyl-4-methoxy-N-[3-(2-methoxyethoxy)-5-(3-methoxypropyl)benzyl]piperidine-3-carboxamide,
Rac-(3S,4R)-4-(2-butoxypyridin-4-yl)-N-cyclopropyl-4-methoxy-N-[3-(2-methoxyethoxy)-5-(3-methoxypropyl)benzyl]piperidine-3-carboxamide,
(3S,4R)—N-cyclopropyl-4-(3,4-difluorophenyl)-4-methoxy-N-[3-(2-methoxyethoxy)-5-(3-methoxypropyl)benzyl]piperidine-3-carboxamide,
(3S,4R)—N-cyclopropyl-4-(3,4-difluorophenyl)-4-ethoxy-N-[3-(2-methoxyethoxy)-5-(3-methoxypropyl)benzyl]piperidine-3-carboxamide,
(3S,4R)—N-cyclopropyl-4-(3,4-difluorophenyl)-4-[(4-fluorobenzyl)oxy]-N-[3-(2-methoxyethoxy)-5-(3-methoxypropyl)benzyl]piperidine-3-carboxamide,
(3S,4R)—N-cyclopropyl-4-(3,4-difluorophenyl)-4-(2-methoxyethoxy)-N-[3-(2-methoxyethoxy)-5-(3-methoxypropyl)benzyl]piperidine-3-carboxamide,
(3S,4R)—N-cyclopropyl-4-(3,4-difluorophenyl)-4-(2,3-dihydroxypropoxy)-N-[3-(2-methoxyethoxy)-5-(3-methoxypropyl)benzyl]piperidine-3-carboxamide trifluoroacetate,
(3S,4R)—N-cyclopropyl-4-(3,4-difluorophenyl)-N-[3-(2-methoxyethoxy)-5-(3-methoxypropyl)benzyl]-4-(1H-1,2,3-triazol-5-ylmethoxy)piperidine-3-carboxamide,
(3S,4R)—N-cyclopropyl-4-methoxy-N-[3-(2-methoxyethoxy)-5-(3-methoxypropyl)benzyl]-4-(1-methyl-2-oxo-1,2-dihydropyridin-4-yl)piperidine-3-carboxamide,
(3S,4R)-4-(2-butoxypyridin-4-yl)-N-cyclopropyl-4-methoxy-N-[3-(2-methoxyethoxy)-5-(3-methoxypropyl)benzyl]piperidine-3-carboxamide, and
(3S,4R)—N-[2-chloro-5-(2-methoxyethyl)benzyl]-N-cyclopropyl-4-(3,4-difluorophenyl)-4-(2,3-dihydroxypropoxy)piperidine-3-carboxamide.

9. A pharmaceutical composition comprising an effective amount of a compound according to claim 1, or a pharmaceutically acceptable salt thereof, and a pharmaceutically acceptable carrier.

10. Use of a compound according to claim 1, or a composition according to claim 9, for the manufacture of a medicament for the treatment or prophylaxis of diseases which are related to hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, glomerulonephritis, renal colic, complications resulting from diabetes such as nephropathy, vasculopathy and neuropathy, glaucoma, elevated intra-ocular pressure, atherosclerosis, restenosis post angioplasty, complications following vascular or cardiac surgery, erectile dysfunction, hyperaldosteronism, lung fibrosis, scleroderma, anxiety, cognitive disorders, complications of treatments with immunosuppressive agents, and other diseases known to be related to the renin-angiotensin system.

11. A method for the treatment or prophylaxis of diseases which are related to hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, glomerulonephritis, renal colic, complications resulting from diabetes such as nephropathy, vasculopathy and neuropathy, glaucoma, elevated intra-ocular pressure, atherosclerosis, restenosis post angioplasty, complications following vascular or cardiac surgery, erectile dysfunction, hyperaldosteronism, lung fibrosis, scleroderma, anxiety, cognitive disorders, complications of treatments with immunosuppressive agents, and other diseases known to be related to the renin-angiotensin system, comprising the administration to a patient of a pharmaceutically active amount of a compound according to claim 1.
